SEQUENCE LISTING (47 sequences)

SEQ ID NO: 1 - protein, 573 aa, synthetic construct
YPYDVPDYAGAQPARSDIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIKGGGSGGGSGGGSGGGSEVQLQQSGPELVKPGASMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGAGTTVTVSSGGGGSGGGGSGGGGSQVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAGGGGSGGGGSGGGGSDILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKGGGGSVDAVGQDTQEVIVVPHSLPFKVVVISAILALVVLTIISLIILIMLWQKKPR

SEQ ID NO: 2 - DNA, 1754 bp, synthetic construct
  source 1..1754: mol_type = other DNA
atccatatgatgttccagattatgctggggcccagccggccagatctgatatccagatgacacagacaacctcaagtcttagtgcatcactgggagatcgtgtgactataagctgccgcgcatcacaggacattcgcaattatctgaattggtatcaacagaagcctgatggcaccgtgaaacttctgatctattacaccagtcgtctgcatagcggtgttccgagcaaattttcaggctcagggtcaggaaccgattattcactgacgattagtaatttagaacaagaagatattgcaacctatttctgtcaacagggtaataccctgccgtggacctttgcaggtggtaccaaactggaaattaaaggaggtggcagtggagggggaagcggcggcggttcaggaggcggttctgaggtccagttacagcagagcggtccggaactggttaaaccgggtgcaagcatgaaaattagctgtaaagcaagcggttatagctttaccggttataccatgaattgggttaaacagagccatggtaaaaatctggaatggatgggtctgattaatccgtataaaggtgttagcacctataatcagaaatttaaagataaagcaaccctgaccgttgataaaagcagcagcaccgcatatatggaactgctgagcctgaccagcgaagatagcgccgtttactattgcgcacgcagcggttattatggtgatagcgattggtattttgatgtttggggtgcaggtaccaccgttaccgttagcagcggaggtggcggaagtggaggaggtggctctggcggtggaggaagccaggtgcagctgaagcagtctggccctggactggtgcagcctagccagagcctgagcatcacctgtaccgtgtccggcttcagcctgaccaactacggcgtgcactgggtgcgacagagccctggcaaaggcctggaatggctgggagtgatttggagcggcggcaacaccgactacaacacccccttcaccagcagactgtccatcaacaaggacaacagcaagagccaggtgttcttcaagatgaacagcctgcagagcaacgacaccgccatctactactgcgctagagccctgacctactatgactacgagttcgcctactggggccagggcacactcgtgacagtgtctgccggcggaggtggatctggaggcggtggcagcggtggaggcggatctgacatcctgctgacccagagccccgtgatcctgtccgtgtctcctggcgagagagtgtccttcagctgcagagccagccagagcatcggcaccaacatccactggtatcagcagaggaccaacggcagccccagactgctgattaagtacgccagcgagtccatcagcggcatccccagcagattcagcggcagcggctctggcaccgacttcaccctgagcatcaacagcgtggaaagcgaggatatcgccgactactactgccagcagaacaacaactggcccaccaccttcggcgctggcaccaagctggaactgaagggcgggggcggaagcgtcgacgaacaaaaactcatctcagaagaggatctgaatgctgtgggccaggacacgcaggaggtcatcgtggtgccacactccttgccctttaaggtggtggtgatctcagccatcctggccctggtggtgctcaccatcatctcccttatcatcctcatcatgctttggcagaagaagccacgttag

SEQ ID NO: 3 - protein, 715 aa, synthetic construct
YPYDVPDYAGAQPARSFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPLASGGGGSPVKGGTKCIKYLLFGFNFIFWLAGIAVLAIGLWLRFDSQTKSIFEQETNNNNSSFYTGVYILIGAGALMMLVGFLGCCGAVQESQCMLGLFFGFLLVIFAIEIAAAIWGYSHKDEVIKEVQEFYKDTYNKLKTKDEPQRETLKAIHYALNCCGLAGGVEQFISDICPKKDVLETFTVKSCPDAIKEVFDNKFHIIGAVGIGIAVVMIFGMIFSMILCCAIRRNREMVGGGGSGGGGSVDERVQPLEENVGNAARPRFERNKLLLVASVIQGLGLLLCFTYICLHFSALQVSHRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVLHHHHHH

SEQ ID NO: 4 - DNA, 2148 bp, synthetic construct
  source 1..2148: mol_type = other DNA
tatccatatgatgttccagattatgctggggcccagccggccagatctttcttagactccccagacaggccctggaacccccccaccttctccccagccctgctcgtggtgaccgaaggggacaacgccaccttcacctgcagcttctccaacacatcggagagcttcgtgctaaactggtaccgcatgagccccagcaaccagacggacaagctggccgctttccccgaggaccgcagccagcccggccaggactgccgcttccgtgtcacacaactgcccaacgggcgtgacttccacatgagcgtggtcagggcccggcgcaatgacagcggcacctacctctgtggggccatctccctggcccccaaggcgcagatcaaagagagcctgcgggcagagctcagggtgacagagagaagggcagaagtgcccacagcccaccccagcccctcacccaggccagccggccagttccaaaccctggtggttggtgtcgtgggcggcctgctgggcagcctggtgctgctagtctgggtcctggccgtcatctgctcccgggccgcacgagggacaataggagccaggcgcaccggccagcccctgaaggaggacccctcagccgtgcctgtgttctctgtggactatggggagctggatttccagtggcgagagaagaccccggagccccccgtgccctgtgtccctgagcagacggagtatgccaccattgtctttcctagcggaatgggcacctcatcccccgcccgcaggggctcagccgacggccctcggagtgcccagccactgaggcctgaggatggacactgctcttggcccctcgctagcggaggcggtggtagtccggtcaaaggaggcaccaagtgcatcaaatacctgctgttcggatttaacttcatcttctggcttgccgggattgctgtccttgccattggactatggctccgattcgactctcagaccaagagcatcttcgagcaagaaactaataataataattccagcttctacacaggagtctatattctgatcggagccggcgccctcatgatgctggtgggcttcctgggctgctgcggggctgtgcaggagtcccagtgcatgctgggactgttcttcggcttcctcttggtgatattcgccattgaaatagctgcggccatctggggatattcccacaaggatgaggtgattaaggaagtccaggagttttacaaggacacctacaacaagctgaaaaccaaggatgagccccagcgggaaacgctgaaagccatccactatgcgttgaactgctgtggtttggctgggggcgtggaacagtttatctcagacatctgccccaagaaggacgtactcgaaaccttcaccgtgaagtcctgtcctgatgccatcaaagaggtcttcgacaataaattccacatcatcggcgcagtgggcatcggcattgccgtggtcatgatatttggcatgatcttcagtatgatcttgtgctgtgctatccgcaggaaccgcgagatggtcggaggcggtggtagcggtggaggtgggtccgtcgacgaaagggtccaacccctggaagagaatgtgggaaatgcagccaggccaagattcgagaggaacaagctattgctggtggcctctgtaattcagggactggggctgctcctgtgcttcacctacatctgcctgcacttctctgctcttcaggtatcacatcggtatcctcgaatacaaagtatcaaagtacaatttaccgaatataagaaggagaaaggtttcatcctcacttcccaaaaggaggatgaaatcatgaaggtgcagaacaactcagtcatcatcaactgtgatgggttttatctcatctccctgaagggctacttctcccaggaagtcaacattagccttcattaccagaaggatgaggagcccctcttccaactgaagaaggtcaggtctgtcaactccttgatggtggcctctctgacttacaaagacaaagtctacttgaatgtgaccactgacaatacctccctggatgacttccatgtgaatggcggagaactgattcttatccatcaaaatcctggtgagttctgtgtccttcaccatcaccaccaccattaa

SEQ ID NO: 5 - protein, 245 aa, synthetic construct
DIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIKGGGSGGGSGGGSGGGSEVQLQQSGPELVKPGASMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGAGTTVTVSS

SEQ ID NO: 6 - protein, 241 aa, synthetic construct
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAGGGGSGGGGSGGGGSDILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELK

SEQ ID NO: 7 - protein, 49 aa, Homo sapiens
AVGQDTQEVIVVPHSLPFKVVVISAILALVVLTIISLIILIMLWQKKPR

SEQ ID NO: 8 - protein, 288 aa, Homo sapiens
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 9 - protein, 176 aa, Homo sapiens
MRIFAVFIFMTYWHLLNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 10 - protein, 273 aa, Homo sapiens
MIFLLLMLSLELQLHQIAALFTVTVPKELYIIEHGSNVTLECNFDTGSHVNLGAITASLQKVENDTSPHRERATLLEEQLPLGKASFHIPQVQVRDEGQYQCIIIYGVAWDYKYLTLKVKASYRKINTHILKVPETDEVELTCQATGYPLAEVSWPNVSVPANTSHSRTPEGLYQVTSVLRLKPPPGRNFSCVFWNTHVRELTLASIDLQSQMEPRTHPTWLLHIFIPFCIIAFIFIATVIALRKQLCQKLYSSKDTTKRPVTTTKREVNSAI

SEQ ID NO: 11 - protein, 228 aa, Homo sapiens
MPVKGGTKCIKYLLFGFNFIFWLAGIAVLAIGLWLRFDSQTKSIFEQETNNNNSSFYTGVYILIGAGALMMLVGFLGCCGAVQESQCMLGLFFGFLLVIFAIEIAAAIWGYSHKDEVIKEVQEFYKDTYNKLKTKDEPQRETLKAIHYALNCCGLAGGVEQFISDICPKKDVLETFTVKSCPDAIKEVFDNKFHIIGAVGIGIAVVMIFGMIFSMILCCAIRRNREMV

SEQ ID NO: 12 - protein, 1091 aa, Homo sapiens
MRPSGTAGAALLALLAALCPASRALEEKKVCQGTSNKLTQLGTFEDHFLSLQRMFNNCEVVLGNLEITYVORNYDLSFLKTIQEVAGYVLIALNTVERIPLENLQIIRGNMYYENSYALAVLSNYDANKTGLKELPMRNLQGQKCDPSCPNGSCWGAGEENCOKLTKIICAQQCSGRCRGKSPSDCCHNQCAAGCTGPRESDCLVCRKFRDEATCKDTCPPLMLYNPTTYQMDVNPEGKYSFGATCVKKCPRNYVVTDHGSCVRACGADSYEMEEDGVRKCKKCEGPCRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFMRRRHIVRKRTLRRLLQERELVEPLTPSGEAPNQALLRILKETEFKKIKVLGSGAFGTVYKGLWIPEGEKVKIPVAIKELREATSPKANKEILDEAYVMASVDNPHVCRLLGICLTSTVQLITQLMPFGCLLDYVREHKDNIGSQYLLNWCVQIAKGMNYLEDRRLVHRDLAARNVLVKTPQHVKITDFGLAKLLGAEEKEYHAEGGKVPIKWMALESILHRIYTHQSDVWSYGVTVWELMTFGSKPYDGIPASEISSILEKGERLPQPPICTIDVYMIMVKCWMIDADSRPKFRELIIEFSKMARDPQRYLVIQGDERMHLPSPTDSNFYRALMDEEDMDDVVDADEYLIPQOGFFSSPSTSRTPLLSSLSATSNNSTVACIDRNGLQSCPIKEDSFLORYSSDPTGALTEDSIDDTFLPVPGEWLVWKQSCSSTSSTHSAAASLQCPSQVLPPASPEGETVADLQTQ

SEQ ID NO: 13 - protein, 277 aa, Homo sapiens
MCVGARRLGRGPCAALLLLGLGLSTVTGLHCVGDTYPSNDRCCHECRPGNGMVSRCSRSQNTVCRPCGPGFYNDVVSSKPCKPCTWCNLRSGSERKQLCTATQDTVCRCRAGTQPLDSYKPGVDCAPCPPGHFSPGDNQACKPWTNCTLAGKHTLQPASNSSDAICEDRDPPATQPQETQGPPARPITVQPTEAWPRTSQGPSTRPVEVPGGRAVAAILGLGLVLGLLGPLAILLALYLLRRDORLPPDAHKPPGGGSFRTPIQEEQADAHSTLAKI

SEQ ID NO: 14 - protein, 183 aa, Homo sapiens
MERVQPLEENVGNAARPRFERNKLLLVASVIQGLGLLLCFTYICLHFSALQVSHRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 15 - protein, 171 aa, Homo sapiens
MEHSTFLSGLVLATLLSQVSPFKIPIEELEDRVFVNCNTSITWVEGTVGTLLSDITRLDLGKRILDPRGIYRCNGTDIYKDKESTVOVHYRMCQSCVELDPATVAGIIVTDVIATLLLALGVFCFAGHETGRLSGAADTQALLRNDQVYQPLRDRDDAQYSHLGGNWARNK

SEQ ID NO: 16 - protein, 207 aa, Homo sapiens
MQSGTHWRVLGLCLLSVGVWGQDGNEEMGGITQTPYKVSISGTTVILTCPQYPGSEILWOHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRORGONKERPPPVPNPDYEPIRKGQRDLYSGLNQRRI

SEQ ID NO: 17 - protein, 184 aa, Homo sapiens
MEQGKGLAVLILAIILLOGTLAQSIKGNHLVKVYDYQEDGSVLLTCDAEAKNITWFKDGKMIGFLTEDKKKWNLGSNAKDPRGMYQCKGSQNKSKPLQVYYRIVICONCIELNAATISGFLFAEIVSIFVLAVGVYFIAGQDGVRQSRASDKQTLLPNDOLYQPLKDREDDOYSHLQGNQLRRN

SEQ ID NO: 18 - protein, 459 aa, Homo sapiens
MNRGVPFREILLLVLQLALLPAATQGKKVVLGKKGDTVELTCTASQKKSIQFHWKNSNQIKILGNQGSFLTKGPSKLNDRADSRRSLWDQGNFPLIIKNLKIEDSDTYICEVEDQKEEVQLLVFGLTANSDTHLLQGQSLTLTLESPPGSSPSVQCRSPRGKNIQGGKTLSVSQLELQDSGTWTCTVLQNQKKVEFKIDIVVLAFQKASSIVYKKEGEQVEFSFPLAFTVEKLTGSGELWWQAERASSSKSWITFDLKNKEVSVKRVTQDPKLQMGKKLPLHLTLPQALPQYAGSGNLTLALEAKTGKLHQEVNLVVMRATQLQKNLTCEVWGPTSPKLMLSLKLENKEAKVSKREKAVWVLNPEAGMWQCLLSDSGQVLLESNIKVLPTWSTPVQPMALIVLGGVAGLLLFIGLGIFFCVRCRHRRRQAERMSQIKRLLSEKKTCQCPHRFQKTCSPI

SEQ ID NO: 19 - protein, 235 aa, Homo sapiens
MALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRRRVCKCPRPVVKSGDKPSLSARYV

SEQ ID NO: 20 - protein, 198 aa, Homo sapiens
MRPRLWLLLAAQLTVLHGNSVLQQTPAYIKVQTNKMVMLSCEAKISLSNMRIYWLRQRQAPSSDSHHEFLALWDSAKGTIHGEEVEQEKIAVFRDASRFILNLTSVKPEDSGIYFCMIVGSPELTFGKGTQLSVVDFLPTTAQPTKKSTLKKRVCRLPRPETQKGLKGKVYQEPLSPNACMDTTAILQPHRSCLTHGS

SEQ ID NO: 21 - protein, 254 aa, Homo sapiens
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLFGSKNVSSETVNITITQGLAVSTISSFFPPGYQVSFCLVMVLLFAVDTGLYFSVKTNIRSSTRDWKDHKFKWRKDPQDK

SEQ ID NO: 22 - protein, 233 aa, Homo sapiens
MWQLLLPTALLLLVSAGMRTEDLPKAVVFLEPQWYSVLEKDSVTLKCQGAYSPEDNSTQWFHNENLISSQASSYFIDAATVNDSGEYRCQTNLSTLSDPVQLEVHIGWLLLQAPRWVFKEEDPIHLRCHSWKNTALHKVTYLQNGKDRKYFHHNSDFHIPKATLKDSGSYFCRGLVGSKNVSSETVNITITQGLAVSTISSFSPPGYQVSFCLVMVLLFAVDTGLYFSVKTNI

SEQ ID NO: 23 - protein, 200 aa, Homo sapiens
MKSGLWYFFLFCLRIKVLTGEINGSANYEMFIFEINGGVQILCKYPDIVQQFKMQLLKGGQILCDLTKTKGSGNTVSIKSLKFCHSQLSNNSVSFFLYNLDHSHANYYFCNLSIFDPPPFKVTLTGGYLHIYESQLCCQLKFWLPIGCAAFVVVCILGCILICWLTKKKYSSSVHDPNGEYMFMRAVNTAKKSRLTDVTL

SEQ ID NO: 24 - protein, 255 aa, Homo sapiens
MGNSCYNIVATLLLVLNFERTRSLQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQIISFFLALTSTALLFLLFFLTLRFSVVKRGRKKLLYIFKQPFMRPVQTTQEEDGCSCRFPEEEEGGCEL

SEQ ID NO: 25 - protein, 526 aa, Homo sapiens
MWEAQFLGLLFLQPLWVAPVKPLQPGAEVPVVWAQEGAPAQLPCSPTIPLQDLSLLRRAGVTWQHQPDSGPPAAAPGHPLAPGPHPAAPSSWGPRPRRYTVLSVGPGGLRSGRLPLQPRVQLDERGRQRGDFSLWLRPARRADAGEYRAAVHLRDRALSCRLRLRLGQASMTASPPGSLRASDWVILNCSFSRPDRPASVHWFRNRGQGRVPVRESPHEIHLAESFLFLPQVSPMDSGPWGCILTYRDGFNVSIMYNLTVLGLEPPTPLTVYAGAGSRVGLPCRLPAGVGTRSFLTAKWTPPGGGPDLLVTGDNGDFTLRLEDVSQAQAGTYTCHIHLQEQQLNATVTLAIITVTPKSFGSPGSLGKLLCEVTPVSGQERFVWSSLDTPSQRSFSGPWLEAQEAQLLSQPWQCQLYQGERLLGAAVYFTELSSPGAQRSGRAPGALPAGHLLLFLILGVLSLLLLVTGAFGFHLWRRQWRPRRFSALEQGIHPPQAQSKIEELEQEPEPEPEPEPEPEPEPEPEQL

SEQ ID NO: 26 - protein, 174 aa, Homo sapiens
MACLGFQRHKAQLNLATRTWPCTLLFFLLFIPVFCKAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIAKEKKPSYNRGLCENAPNRARM

SEQ ID NO: 27 - protein, 275 aa, Homo sapiens
MWIDFFTYSSMSEEVTYADLQFQNSSEMEKIPEIGKFGEKAPPAPSHVWRPAALFLTLLCLLLLIGLGVLASMEHVTLKIEMKKMNKLQNISEELQRNISLQLMSNMNISNKIRNLSTTLQTIATKLCRELYSKEQEHKCKPCPRRWIWHKDSCYFLSDDVQTWQESKMACAAQNASLLKINNKNALEFIKSQSRSYDYWLGLSPEEDSTRGMRVDNIINSSAWVIRNAPDLNNMYCGYINRLYVQYYHCTYKKRMICEKMANPVQLGSTYFREA

SEQ ID NO: 28 - protein, 1225 aa, Homo sapiens
MKLRLPASPETHLDMLRHLYQGCQVVQGNLELTYLPTNASLSFLQDIQEVQGYVLIAHNQVRQVPLQRLRIVRGTQLFEDNYALAVLDNGDPLNNTTPVTGASPGGLRELQLRSLTEILKGGVLIQRNPQLCYQDTILWKDIFHKNNQLALTLIDTNRSRACHPCSPMCKGSRCWGESSEDCQSLTRTVCAGGCARCKGPLPTDCCHEQCAAGCTGPKHSDCLACLHFNHSGICELHCPALVTYNTDTFESMPNPEGRYTFGASCVTACPYNYLSTDVGSCTLVCPLHNQEVTAEDGTQRCEKCSKPCARVCYGLGMEHLREVRAVTSANIQEFAGCKKIFGSLAFLPESFDGDPASNTAPLQPEQLQVFETLEEITGYLYISAWPDSLPDLSVFQNLQVIRGRILHNGAYSLTLQGLGISWLGLRSLRELGSGLALIHENTHLCFVHTVPWDQLFRNPHQALLHTANRPEDECVGEGLACHQLCARGHCWGPGPTQCVNCSQFLRGQECVEECRVLQGLPREYVNARHCLPCHPECQPQNGSVTCFGPEADQCVACAHYKDPPFCVARCPSGVKPDLSYMPIWKFPDEEGACQPCPINCTHSCVDLDDKGCPAEQRASPLTSIISAVVGILLVVVLGVVFGILIKRRQQKIRKYTMRRLLQETELVEPLTPSGAMPNQAQMRILKETELRKVKVLGSGAFGTVYKGIWIPDGENVKIPVAIKVLRENTSPKANKEILDEAYVMAGVGSPYVSRLLGICLTSTVQLVTQLMPYGCLLDHVRENRGRLGSQDLLNWCMQIAKGMSYLEDVRLVHRDLAARNVLVKSPNHVKITDFGLARLLDIDETEYHADGGKVPIKWMALESILRRRFTHQSDVWSYGVTVWELMTFGAKPYDGIPAREIPDLLEKGERLPQPPICTIDVYMIMVKCWMIDSECRPRFRELVSEFSRMARDPQRFVVIQNEDLGPASPLDSTFYRSLLEDDDMGDLVDAEEYLVPQQGFFCPDPAPGAGGMVHHRHRSSSTRSGGGDLTLGLEPSEEEAPRSPLAPSEGAGSDVFDGDLGMGAAKGLQSLPTHDPSPLQRYSEDPTVPLPSETDGYVAPLTCSPQPEYVNQPDVRPQPPSPREGPLPAARPAGATLERPKTLSPGKNGVVKDVFAFGGAVENPEYLTPQGGAAPQPHPPPAFSPAFDNLYYWDQDPPERGAPPSTFKGTPTAENPEYLGLDVPV

SEQ ID NO: 29 - protein, 236 aa, Homo sapiens
MPLLLLLPLLWADLTHRPKILIPGTLEPGHSKNLTCSVSWACEQGTPPIFSWLSAAPTSLGPRTTHSSVLIITPRPQDHGTNLTCQVKFAGAGVTTERTIQLNVTYVPQNPTTGIFPGDGSKQETRAGVVHGAIGGAGVTALLALCLCLIFFIVKTHRRKAARTAVGRNDTHPTTGSASPKHQKKSKLHGPTETSSCSGAAPTVEMDEELHYASLNFHGMNPSKDTSTEYSEVRTQ

SEQ ID NO: 30 - protein, 300 aa, Homo sapiens
MANCEFSPVSGDKPCCRLSRRAQLCLGVSILVLILVVVLAVVVPRWRQQWSGPGTTKRFPETVLARCVKYTEIHPEMRHVDCQSVWDAFKGAFISKHPCNITEEDYQPLMKLGTQTVPCNKILLWSRIKDLAHQFTQVQRDMFTLEDTLLGYLADDLTWCGEFNTSKINYQSCPDWRKDCSNNPVSVFWKTVSRRFAEAACDVVHVMLNGSRSKIFDKNSTFGSVEVHNLQPEKVQTLEAWVIHGGREDSRDLCQDPTIKELESIISKRNIQFSCKNIYRPDKFLQCVKNPEDSSCTSEI

SEQ ID NO: 31 - protein, 300 aa, Homo sapiens
MVLLWLTLLLIALPCLLQTKEGGKPWAGAENLTCWIHDVDFLSCSWAVGPGAPADVQYDLYLNVANRRQQYECLHYKTDAQGTRIGCRFDDISRLSSGSQSSHILVRGRSAAFGIPCTDKFVVFSQIEILTPPNMTAKCNKTHSFMHWKMRSHFNRKFRYELQIQKRMQPVITEQVRDRTSFQLLNPGTYTVQIRARERVYEFLSAWSTPQRFECDQEEGANTRAWRTSLLIALGTLLALVCVFVICRRYLVMQRLFPRIPHMKDPIGDSFQNDKLVVWEAGKAGLEECLVTEVQVVQKT

SEQ ID NO: 32 - protein, 301 aa, Homo sapiens
MFSHLPFDCVLLLLLLLLTRSSEVEYRAEVGQNAYLPCFYTPAAPGNLVPVCWGKGACPVFECGNVVLRTDERDVNYWTSRYWLNGDFRKGDVSLTIENVTLADSGIYCCRIQIPGIMNDEKFNLKLVIKPAKVTPAPTRQRDFTAAFPRMLTTRGHGPAETQTLGSLPDINLTQISTLANELRDSRLANDLRDSGATIRIGIYIGAGICAGLALALIFGALIFKWYSHSKEKIQNLSLISLANLPPSGLANAVAEGIRSEENIYTIEENVYEVEEPNEYYCYVSSRQQPSQPLGCRFAMP

SEQ ID NO: 33 - protein, 10 aa, synthetic construct
GGGGSGGGGS

SEQ ID NO: 34 - protein, 4 aa, synthetic construct
GGGS

SEQ ID NO: 35 - protein, 16 aa, synthetic construct
GGGSGGGSGGGSGGGS

SEQ ID NO: 36 - protein, 5 aa, synthetic construct
GGGGS

SEQ ID NO: 37 - protein, 15 aa, synthetic construct
GGGGSGGGGSGGGGS

SEQ ID NO: 38 - protein, 10 aa, synthetic construct
EQKLISEEDL

SEQ ID NO: 39 - protein, 6 aa, synthetic construct
HHHHHH

SEQ ID NO: 40 - protein, 9 aa, synthetic construct
YPYDVPDYA

SEQ ID NO: 41 - DNA, 34 bp, synthetic construct
  source 1..34: mol_type = other DNA
cagtgtgctggaattcggcttggggatatccacc

SEQ ID NO: 42 - DNA, 50 bp, synthetic construct
  source 1..50: mol_type = other DNA
ccggactaccaccgcctccgctagcgaggggccaagagcagtgtccatcc

SEQ ID NO: 43 - DNA, 60 bp, synthetic construct
  source 1..60: mol_type = other DNA
ccctcgctagcggaggcggtggtagtccggtcaaaggaggcaccaagtgcatcaaatacc

SEQ ID NO: 44 - DNA, 42 bp, synthetic construct
  source 1..42: mol_type = other DNA
gatctcgagcggccgccttaatggtggtggtgatggtgaagg

SEQ ID NO: 45 - DNA, 34 bp, synthetic construct; identical to SEQ ID NO: 41
  source 1..34: mol_type = other DNA

SEQ ID NO: 46 - DNA, 42 bp, synthetic construct; identical to SEQ ID NO: 44
  source 1..42: mol_type = other DNA

SEQ ID NO: 47 - protein, 21 aa, synthetic construct
METDTLLLWVLLLWVPGSTGD

(19) United States
(12) Patent Application Publication
(10) Pub. No.: US 2025/0092143 A1
(43) Pub. Date: Mar. 20, 2025

(54) GENETICALLY ENGINEERED MULTIFUNCTIONAL EXOSOMES FOR IMMUNOTHERAPY

(71) Applicant: UNIVERSITY OF SOUTHERN CALIFORNIA, Los Angeles, CA (US)

(72) Inventors: Yong ZHANG, Los Angeles, CA (US); Qinqin CHENG, Los Angeles, CA (US)

(73) Assignee: UNIVERSITY OF SOUTHERN CALIFORNIA, Los Angeles, CA (US)

(21) Appl. No.: 18/580,889

(22) PCT Filed: Jul. 22, 2022
(86) PCT No.: PCT/US2022/074060

§ 371 Date: Jan. 19, 2024

Related U.S. Application Data
(60) Provisional application No. 63/225,179, filed on Jul. 23, 2021.

(51) Int. Cl. C07K 16/28; A61K 9/50; A61K 35/00; A61K 38/00; C07K 14/705; C07K 14/71

(52) U.S. Cl. CPC C07K 16/2863; A61K 9/5068; A61K 35/00; C07K 14/70521; C07K 14/70575; C07K 14/71; C07K 16/2809; A61K 38/00; C07K 2317/31; C07K 2317/622; C07K 2319/03; C07K 2319/21; C07K 2319/42

(57) ABSTRACT

The disclosure provides for an engineered extracelluar vesicle and methods of using the same where the engineered extracellular vesicle comprises a first fusion protein having the formula A-B-C, wherein A is a first antibody moiety, B is a second antibody moiety, and C is a first exosomal protein transmembrane domain; and a second fusion protein comprising the formula D-E-F, wherein D is a first protein binding moiety, E is a second exosomal membrane protein transmembrane domain, and F is a second protein binding moiety; wherein both the first fusion protein and the second fusion protein are displayed on a surface of the engineered extracellular vesicle, and the first antibody moiety and the second antibody moiety separately bind to a an immune cell marker protein and a cancer cell surface-marker protein, and the first binding protein moiety and the second protein binding moiety separately bind to a second immune cell marker protein and a second cancer cell surface-marker protein.

### RELATED APPLICATIONS

This application claims priority under 35 U.S.C. § 119(e) to U.S. Provisional Patent Application No. 63/225,179 filed Jul. 23, 2021, which is incorporated herein by reference.

### GOVERNMENT SUPPORT

This invention was made with government support under Grant Number P30CA014089, awarded by the National Institutes of Health, and Grant Number W81XWH-19-1-0272, awarded by the U.S. Department of Defense. The government has certain rights in the invention.

### BACKGROUND OF THE INVENTION

Extracellular vesicles, and in particular, exosomes are naturally occurring membranous vesicles derived from a variety types of cells. Featured with a diameter of 30-150 nm, exosomes carry a substantial amount of contents from parental cells. Through direct interactions with cell surface receptors and ligands and/or materials transfer via different modes, exosomes are capable of modulating physiology and pathophysiology of recipient cells. These nanoscale vesicles are known as key mediators for short- and long-range cell-to-cell communications.

As a native form of nanocarriers, exosomes possess characteristic lipid bilayers and membrane proteins that constitute important functional components. By promoting and directing membrane fusion to target cells and suppressing phagocytic clearance, proteins on exosome surfaces facilitate cytosolic delivery and increase half-lives in circulation, enhancing exosome's pharmacological properties. Together with excellent biocompatibility, these valuable features draw significant interests in developing exosomes as a new class of nanomedicine.

To date, exosome-aided drug delivery shows broad utility in the treatment of various human diseases. But exosome potential for immunotherapy has yet to be fully leveraged. Accordingly, there is a need for specific, effective, and efficient targeting of target cells by extracellular vesicles, such as exosomes, to cause therapeutic actions initiated by the binding of the extracellular vesicle to the target cells. The present disclosure satisfies these needs.

### SUMMARY OF THE INVENTION

Considering important roles and unique advantages of exosomes in intercellular communications, it is envisioned that functionally reprogramming these cell-derived nanovesicles may result in an innovative form of agents with desired activities in eliciting disease-specific immune responses. In contrast to molecularly defined immunotherapeutics such as immune checkpoint inhibitors and bispecific antibodies, exosomes may enable multivalent expression of immunomodulatory proteins on spherical surface. This will increase their avidity and binding affinity to target receptors or ligands on immune and diseased cells and foster the formation of immunological synapses, resulting in enhanced activation of the immune system. Moreover, functional display of multiple immunomodulatory proteins on the same exosome vesicle, which target different signaling pathways, may promote synergistic actions, offering improved therapeutic efficacy in comparison to conventional combination therapies.

Accordingly, the present disclosure shows not only distinct immune checkpoint modulators but also targeting moieties on exosome surfaces using genetic approaches. The resulting exosomes, named genetically engineered multifunctional immune-modulating exosomes (GEMINI-Exos), (FIG. 1). Some embodiments are characterized by surface-displayed programmed death 1 (PD-1) and OX40 ligand (OX40L) as well as monoclonal antibodies specific for T-cell CD3 and epidermal growth factor receptor (EGFR), a receptor tyrosine kinase frequently overexpressed in many human cancers. The generated αCD3-αEGFR-PD-1-OX40L GEMINI-Exos display strong binding affinity to human CD3, EGFR, PD-1 ligands, and OX40. Preclinical studies using cellular and animal models of triple negative breast cancer (TNBC) indicated αCD3-αEGFR-PD-1-OX40L GEMINI-Exos can induce potent cellular immunity against EGFR-positive TNBC tumors by elevating infiltration of CD8⁺ T cells and alleviating immunosuppression of regulatory T cells (Tregs). This work demonstrates the feasibility of GEMINI-Exos-based cancer immunotherapy and provides a new strategy for developing immunotherapeutic exosomes.

Some embodiments of the present disclosure provide for an engineered extracellular vesicle comprising: a first fusion protein comprising a formula A-B-C, wherein A is a first antibody moiety, B is a second antibody moiety, and C is a first exosomal protein transmembrane domain; and a second fusion protein comprising the formula D-E-F, wherein D is a first protein binding moiety, E is a second exosomal membrane protein transmembrane domain, and F is a second protein binding moiety; wherein both the first fusion protein and the second fusion protein are displayed on a surface of the engineered extracellular vesicle, and the first antibody moiety and the second antibody moiety separately bind to a first immune cell marker protein and a first cancer cell surface-marker protein, and the first protein binding moiety and the second protein binding moiety separately bind to a second immune cell marker protein and a second cancer cell surface-marker protein. Preferably, the extracellular vesicles are exosomes.

In some embodiments, the first antibody moiety and the second antibody moiety are one of a single chain variable fragment (scFv), a single domain antibody, a bispecific antibody, or a multispecific antibody. Preferably, the first and second antibody binding moieties are scFvs.

In some embodiments, the first and second antibody moieties bind separately to an immune cell surface marker protein comprising one or more of CD3, CD2, CD4, CD5, CD7, CD8, CD14, CD15, CD16, CD24, CD25, CD27, CD28, CD30, CD31, CD38, CD40L, CD45, CD56, CD68, CD91, CD114, CD163, CD206, LFA1, PD-1, ICOS, BTLA, KIR, CD137, OX40, LAG3, CTLA4, and a T-cell Receptor, or a cancer cell surface-marker protein comprising one or more of EGFR, CLL-1, HER2, HER3, CD33, CD34, CD38, CD123, TIM3, CD25, CD32, CD96, and PD-L1/L2. In some embodiments, the first antibody moiety binds to T-cell surface-marker protein and the second antibody binds to a cancer cell surface marker protein, or vice versa.

In some embodiments, the engineered extracellular vesicle comprises a first fusion protein comprising a formula T₁-A-L₁-B-L₂-C-T₂, wherein T1 is a first epitope tag, A is the first antibody moiety, L₁ is a first linker moiety, B is the second antibody moiety, L₂ is a second linker moiety, C is the first exosomal protein transmembrane domain, and T₂ is a second epitope tag; and a second fusion protein comprising a formula T₃-D-L3-E-L4-F, wherein T3 is a third epitope tag, D is the first protein binding moiety, L₃ is a third linker moiety, E is the second exosomal membrane protein transmembrane domain, L₄ is a fourth linker moiety, and F is the second protein binding moiety.

The disclosure also provides for a method of treating cancer comprising administering an effective amount any of an engineered extracellular vesicle, or a composition thereof, to a subject having or suspected of having cancer whereby the engineered extracellular vesicles selectively activates T-cells to kill cancer cells, wherein the engineered extracellular vesicle comprises: a first fusion protein comprising a formula A-B-C, wherein A is a first antibody moiety, B is a second antibody moiety, and C is a first exosomal protein transmembrane domain; and a second fusion protein comprising the formula D-E-F, wherein D is a first protein binding moiety, E is a second exosomal membrane protein transmembrane domain, and F is a second protein binding moiety; wherein both the first fusion protein and the second fusion protein are displayed on a surface of the engineered extracellular vesicle, and the first antibody moiety and the second antibody moiety separately bind to a first immune cell marker protein and a first cancer cell surface-marker protein, and the first protein binding moiety and the second protein binding moiety separately bind to a second immune cell marker protein and a second cancer cell surface-marker protein. Preferably, the extracellular vesicles are exosomes and the first protein binding moiety and the second protein binding moiety are not antibodies.

These and other features and advantages of this invention will be more fully understood from the following detailed description of the invention taken together with the accompanying claims. It is noted that the scope of the claims is defined by the recitations therein and not by the specific discussion of features and advantages set forth in the present description.

### BRIEF DESCRIPTION OF THE DRAWINGS

The following drawings form part of the specification and are included to further demonstrate certain embodiments or various aspects of the invention. In some instances, embodiments of the invention can be best understood by referring to the accompanying drawings in combination with the detailed description presented herein. The description and accompanying drawings may highlight a certain specific example, or a certain aspect of the invention. However, one skilled in the art will understand that portions of the example or aspect may be used in combination with other examples or aspects of the invention.

FIG. 1. Schematic of αCD3-αEGFR-PD-1-OX40L GEMINI-Exos for targeted cancer immunotherapy. HA: hemagglutinin; PD-1: programmed death 1; OX40L: OX40 ligand; αCD3: anti-CD3; αEGFR: anti-EGFR; scFv: single-chain variable fragment; PDGFR TMD: transmembrane domain of human platelet-derived growth factor receptor; PD-L1/L2: programmed death-ligand 1/ligand 2.

FIG. 2. Generation and characterization of PD-1-OX40L-Exos. (A) Immunoblot analysis of purified exosomes. (B) Size distribution of native exosomes and PD-1-OX40L-Exos. (C) Sandwich ELISA analysis of the binding of PD-1-OX40L-Exos to human PD-L1 and OX40. Recombinant human PD-L1 and biotinylated OX40 were used as capture and detection reagents, respectively. Data are shown as mean±SD of duplicates. (D)-(F) Flow cytometry of the binding of PD-1-OX40L-Exos to IFN-γ stimulated BT-20 cells (D), activated human T cells (E), and MDA-MB-468 cells (F). Anti-PD-L1, anti-PD-L2, and anti-OX40 antibodies were used as positive controls. Arrows show signal levels of indicated target proteins being detected after the incubation with antibodies, native Exos, or PD-1-OX40L-Exos. (G) and (H) Dose-dependent activation of human T cells by PD-1-OX40L-Exos. Human PBMCs were incubated with pre-coated anti-human CD3 monoclonal antibody in the presence of various concentrations of PD-1-OX40L-Exos or native exosomes for 48 hours. The levels of secreted IFN-γ (G) and IL-2 (H) were measured by ELISA. Data are shown as mean±SD of triplicates. (I) PD-1-OX40L-Exos restore T-cell activation from PD-L1-mediated inhibition. Human PBMCs were incubated with pre-coated anti-human CD3 monoclonal antibody without or with pre-coated human PD-L1 in the absence or presence of 10 μg mL⁻¹ PD-1-OX40L-Exos or native exosomes for 48 hours. The levels of secreted IL-2 were measured by ELISA. Data are shown as mean±SD of triplicates. * P<0.05 and **** P<0.0001 (two-tailed unpaired t test).

FIG. 3. Generation and characterization of αCD3-αEGFR-PD-1-OX40L GEMINI-Exos. (A) Immunoblot analysis of purified exosomes. (B) Size distribution of αCD3-αEGFR-PD-1-OX40L GEMINI-Exos. (C) ELISA analysis of the binding of αCD3-αEGFR-PD-1-OX40L GEMINI-Exos to human PD-L1, PD-L2, and OX40. PD-1-OX40L-Exos, αCD3-αEGFR-PD-1-OX40L GEMINI-Exos, and native exosomes at various concentrations were coated on 96-well ELISA plates overnight, followed by incubation with recombinant PD-L1-Fc, PD-L2-Fc or OX40-Fc and detection with an anti-human IgG-HRP. Data are shown as mean±SD of duplicates. (D) Flow cytometry of the binding of αCD3-αEGFR-PD-1-OX40L GEMINI-Exos to BT-20 cells (EGFR⁺ PD-L1⁺) and Jurkat cells (CD3⁺). Arrows show signal levels of indicated target proteins being detected after the incubation with various types of exosomes. (E) Time-dependent activation of human T cells by αCD3-αEGFR-PD-1-OX40L GEMINI-Exos. Human PBMCs were incubated with BT-20 cells at a ratio of 2:1 for 24-96 hours in the presence of native exosomes, PD-1-OX40L-Exos, αCD3-αEGFR-Exos, a mixture (1:1) of PD-1-OX40L- and αCD3-αEGFR-Exos, or αCD3-αEGFR-PD-1-OX40L GEMINI-Exos. The levels of secreted IL-2 were measured by ELISA. Data are shown as mean±SD of triplicates. ns=not significant, * P<0.05, and **** P <0.0001 (ordinary one-way ANOVA test).

FIG. 4. In vivo evaluation of αCD3-αEGFR-PD-1-OX40L GEMINI-Exos. (A) Anti-tumor activity of GEMINI-Exos. BT-20 cells were subcutaneously implanted into the flank of female NSG mice (n=5). In vitro expanded human PBMCs from the same healthy donor were intraperitoneally injected into mice on days 12 and 18 post tumor implantation. One day post the first PBMCs administration, mice were treated with PBS or different types of exosomes (10 mg/kg for monotherapy and 20 mg/kg for combination therapy) every other day for a total of six times via intravenous injections. Data are shown as mean±SD (n=5). ns=not significant, * P<0.05, and ** P<0.01 (one-way repeated measures ANOVA test with the Geisser-Greenhouse correction). (B) Tumor weights at the end of study. (C) Body weights of mice during the study. (D) ALT activities in plasma at the end of study. (E) Creatinine concentrations in plasma at the end of study. (F) Percentage CD8⁺ T cells in CD45⁺ cells in tumors. (G) Percentages of CD4⁺CD25⁺ FoxP3⁺ Tregs in CD45⁺ cells in tumors. (H) CD8⁺ T-cell/Treg ratios in tumors. At the end of study, tumors were harvested and disaggregated into single-cell suspensions. After immunostainings, cells were analyzed by flow cytometry for the expression of CD45, CD4, CD8, CD25 and FoxP3. (I) Tumor growth curves for individual mice during the study. Data in (B, D-H) are shown as mean±SD (n=5). ns=not significant, * P<0.05, ** P<0.01, *** P<0.001, and **** P<0.0001 (ordinary one-way ANOVA test).

FIG. 5. Flow cytometric analysis of expression levels of PD-L1 and PD-L2 at varied conditions. (A) and (B) Surface expression levels of PD-L1 (A) and PD-L2 (B) for HEK293 and three TNBC cell lines without and with stimulations. HEK293, MDA-MB-231, BT-20, and MDA-MB-468 cells were treated with 100 U/mL IFN-7 or human PBMCs (PBMC:TNBC/HEK293=2:1) in the absence or presence of 20 ng/mL αCD3-αEGFR-Exos for 48 hours at 37° C. Non-treated and treated cells were then analyzed for PD-L1 and PD-L2 expression by flow cytometry. Lower panels: quantitative representations of mean fluorescence intensities (MFIs) of PD-L1 (A) or PD-L2 (B) for each cell line. Data are shown as mean±SD of triplicates. ns=not significant, * P<0.05, ** P<0.01, *** P<0.001, and **** P<0.0001 (ordinary one-way ANOVA test).

FIG. 6. Flow cytometric analysis of OX40 expression on non-activated T cells.

FIG. 7. Enhancing T-cell activation by PD-1-OX40L-Exos. Human PBMCs were mixed with BT-20 cells at a ratio of 2:1 and incubated without or with αCD3-αEGFR-Exos (20 ng mL⁻¹) in the absence or presence of 10 μg mL⁻¹ PD-1-OX40L-Exos or native exosomes for 48 hours. The levels of secreted IL-2 were measured by ELISA. Data are shown as mean±SD of duplicates. * P<0.05 (two-tailed unpaired t test).

FIG. 8. Yields of the genetically modified exosomes. Data are shown as mean±SD (n=4). ns=not significant, P>0.05 (ordinary one-way ANOVA test).

FIG. 9. Flow cytometric analysis of EGFR expression for three TNBC cell lines. Right panel: quantitative representations of MFIs of EGFR for each cell line. Data are shown as mean±SD of triplicates.

FIG. 10. Flow cytometry of the binding of αCD3-αEGFR-PD-1-OX40L GEMINI-Exos to MDA-MB-468 cells (PD-L1⁻ PD-L2⁻ OX40⁻ CD3⁻ EGFR⁺) as detected by the anti-HA or anti-6×His antibody.

FIG. 11. Photographs of xenografted mouse tumors at the endpoint.

FIG. 12. Immune phenotyping of tumor infiltrating lymphocytes. (A) Percentages of CD4⁺ T cells in CD45⁺ cells in tumors. (B) Percentages of CD4⁺CD25⁺CD127⁻ Tregs in CD45⁺ cells in tumors. At the end of the in vivo efficacy study, tumors were harvested and disaggregated into single-cell suspensions. After immunostainings, cells were analyzed by flow cytometry for the expression of CD45, CD4, CD8, CD25, and CD127. Data are shown as mean±SD (n=5). ns=not significant, * P<0.05, and ** P<0.01 (ordinary one-way ANOVA test).

FIG. 13. Immunohistofluorescence analysis of tumor-infiltrating T lymphocytes. Quantitative representation of the number of CD3⁺ cells from each field of view along the margin and interior of each tumor from PBS- and exosomes-treated groups (up to three fields of view per region and three mice per group). Data are shown as mean±SD. * P<0.05, ** P<0.01, *** P<0.001, and **** p<0.0001 (ordinary one-way ANOVA test).

FIG. 14. Immune phenotyping of lymphocytes in spleen and blood. At the end of the in vivo efficacy study, blood and spleen were harvested and spleen samples were disaggregated into single-cell suspensions. After immunostainings, cells were analyzed by flow cytometry for the expression of CD45, CD4, CD8, CD25, and FoxP3. Percentages of CD4⁺ T cells (top), CD8⁺ T cells (middle), and CD4⁺ CD25⁺ FoxP3⁺ Tregs (bottom) in CD45⁺ cells in spleen (left) and blood (right) were determined. Data are shown as mean±SD (n=5). * P<0.05 (ordinary one-way ANOVA test).

### DETAILED DESCRIPTION OF THE INVENTION

### Definitions

The following definitions are included to provide a clear and consistent understanding of the specification and claims. As used herein, the recited terms have the following meanings. All other terms and phrases used in this specification have their ordinary meanings as one of skill in the art would understand. Such ordinary meanings may be obtained by reference to technical dictionaries, such as Hawley's Condensed Chemical Dictionary 14^{th} Edition, by R. J. Lewis, John Wiley & Sons, New York, N.Y., 2001 or Singleton, et al., Dictionary of Microbiology and Molecular Biology, 2d ed., John Wiley and Sons, New York (1994), and Hale & Markham, The Harper Collins Dictionary of Biology. Harper Perennial, N.Y. (1991).

References in the specification to “one embodiment”, “an embodiment”, etc., indicate that the embodiment described may include a particular aspect, feature, structure, moiety, or characteristic, but not every embodiment necessarily includes that aspect, feature, structure, moiety, or characteristic. Moreover, such phrases may, but do not necessarily, refer to the same embodiment referred to in other portions of the specification. Further, when a particular aspect, feature, structure, moiety, or characteristic is described in connection with an embodiment, it is within the knowledge of one skilled in the art to affect or connect such aspect, feature, structure, moiety, or characteristic with other embodiments, whether or not explicitly described.

The singular forms “a,” “an,” and “the” include plural reference unless the context clearly dictates otherwise. Thus, for example, a reference to “a compound” includes a plurality of such compounds, so that a compound X includes a plurality of compounds X. It is further noted that the claims may be drafted to exclude any optional element. As such, this statement is intended to serve as antecedent basis for the use of exclusive terminology, such as “solely,” “only,” and the like, in connection with any element described herein, and/or the recitation of claim elements or use of “negative” limitations.

The term “and/or” means any one of the items, any combination of the items, or all of the items with which this term is associated. The phrases “one or more” and “at least one” are readily understood by one of skill in the art, particularly when read in context of its usage. For example, the phrase can mean one, two, three, four, five, six, ten, 100, or any upper limit approximately 10, 100, or 1000 times higher than a recited lower limit. For example, one or more substituents on a phenyl ring refers to one to five substituents on the ring.

As will be understood by the skilled artisan, all numbers, including those expressing quantities of ingredients, properties such as molecular weight, reaction conditions, and so forth, are approximations and are understood as being optionally modified in all instances by the term “about.” These values can vary depending upon the desired properties sought to be obtained by those skilled in the art utilizing the teachings of the descriptions herein. It is also understood that such values inherently contain variability necessarily resulting from the standard deviations found in their respective testing measurements. When values are expressed as approximations, by use of the antecedent “about,” it will be understood that the particular value without the modifier “about” also forms a further aspect.

The terms “about” and “approximately” are used interchangeably. Both terms can refer to a variation of ±5%, ±10%, ±20%, or ±25% of the value specified. For example, “about 50” percent can in some embodiments carry a variation from 45 to 55 percent, or as otherwise defined by a particular claim. For integer ranges, the term “about” can include one or two integers greater than and/or less than a recited integer at each end of the range. Unless indicated otherwise herein, the terms “about” and “approximately” are intended to include values, e.g., weight percentages, proximate to the recited range that are equivalent in terms of the functionality of the individual ingredient, composition, or embodiment. The terms “about” and “approximately” can also modify the endpoints of a recited range as discussed above in this paragraph.

As will be understood by one skilled in the art, for any and all purposes, particularly in terms of providing a written description, all ranges recited herein also encompass any and all possible sub-ranges and combinations of sub-ranges thereof, as well as the individual values making up the range, particularly integer values. It is therefore understood that each unit between two particular units are also disclosed. For example, if 10 to 15 is disclosed, then 11, 12, 13, and 14 are also disclosed, individually, and as part of a range. A recited range (e.g., weight percentages or carbon groups) includes each specific value, integer, decimal, or identity within the range. Any listed range can be easily recognized as sufficiently describing and enabling the same range being broken down into at least equal halves, thirds, quarters, fifths, or tenths. As a non-limiting example, each range discussed herein can be readily broken down into a lower third, middle third and upper third, etc. As will also be understood by one skilled in the art, all language such as “up to”, “at least”, “greater than”, “less than”, “more than”, “or more”, and the like, include the number recited and such terms refer to ranges that can be subsequently broken down into sub-ranges as discussed above. In the same manner, all ratios recited herein also include all sub-ratios falling within the broader ratio. Accordingly, specific values recited for radicals, substituents, and ranges, are for illustration only; they do not exclude other defined values or other values within defined ranges for radicals and substituents. It will be further understood that the endpoints of each of the ranges are significant both in relation to the other endpoint, and independently of the other endpoint.

This disclosure provides ranges, limits, and deviations to variables such as volume, mass, percentages, ratios, etc. It is understood by an ordinary person skilled in the art that a range, such as “number 1” to “number 2”, implies a continuous range of numbers that includes the whole numbers and fractional numbers. For example, 1 to 10 means 1, 2, 3, 4, 5, . . . 9, 10. It also means 1.0, 1.1, 1.2. 1.3, . . . , 9.8, 9.9, 10.0, and also means 1.01, 1.02, 1.03, and so on. If the variable disclosed is a number less than “number10”, it implies a continuous range that includes whole numbers and fractional numbers less than number10, as discussed above. Similarly, if the variable disclosed is a number greater than “number10”, it implies a continuous range that includes whole numbers and fractional numbers greater than number10. These ranges can be modified by the term “about”, whose meaning has been described above.

One skilled in the art will also readily recognize that where members are grouped together in a common manner, such as in a Markush group, the invention encompasses not only the entire group listed as a whole, but each member of the group individually and all possible subgroups of the main group. Additionally, for all purposes, the invention encompasses not only the main group, but also the main group absent one or more of the group members. The invention therefore envisages the explicit exclusion of any one or more of members of a recited group. Accordingly, provisos may apply to any of the disclosed categories or embodiments whereby any one or more of the recited elements, species, or embodiments, may be excluded from such categories or embodiments, for example, for use in an explicit negative limitation.

The term “contacting” refers to the act of touching, making contact, or of bringing to immediate or close proximity, including at the cellular or molecular level, for example, to bring about a physiological reaction, a chemical reaction, or a physical change, e.g., in a solution, in a reaction mixture, in vitro, or in vivo.

An “effective amount” refers to an amount effective to treat a disease, disorder, and/or condition, or to bring about a recited effect. For example, an effective amount can be an amount effective to reduce the progression or severity of the condition or symptoms being treated. Determination of a therapeutically effective amount is well within the capacity of persons skilled in the art. The term “effective amount” is intended to include an amount of a compound described herein, or an amount of a combination of compounds described herein, e.g., that is effective to treat or prevent a disease or disorder, or to treat the symptoms of the disease or disorder, in a host. Thus, an “effective amount” generally means an amount that provides the desired effect.

Alternatively, the terms “effective amount” or “therapeutically effective amount,” as used herein, refer to a sufficient amount of an agent or a composition or combination of compositions being administered which will relieve to some extent one or more of the symptoms of the disease or condition being treated. The result can be reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. An appropriate “effective” amount in any individual case may be determined using techniques, such as a dose escalation study. The dose could be administered in one or more administrations. However, the precise determination of what would be considered an effective dose may be based on factors individual to each patient, including, but not limited to, the patient's age, size, type or extent of disease, stage of the disease, route of administration of the compositions, the type or extent of supplemental therapy used, ongoing disease process and type of treatment desired (e.g., aggressive vs. conventional treatment).

As used herein, “subject” or “patient” means an individual having symptoms of, or at risk for, a disease or other malignancy. A patient may be human or non-human and may include, for example, animal strains or species used as “model systems” for research purposes, such a mouse model as described herein. Likewise, patient may include either adults or juveniles (e.g., children). Moreover, patient may mean any living organism, preferably a mammal (e.g., human or non-human) that may benefit from the administration of compositions contemplated herein. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish and the like. In one embodiment of the methods provided herein, the mammal is a human.

As used herein, the terms “providing”, “administering,” “introducing,” are used interchangeably herein and refer to the placement of a compound of the disclosure into a subject by a method or route that results in at least partial localization of the compound to a desired site. The compound can be administered by any appropriate route that results in delivery to a desired location in the subject.

The compounds and compositions described herein may be administered with additional compositions to prolong stability and activity of the compositions, or in combination with other therapeutic drugs.

The terms “inhibit”, “inhibiting”, and “inhibition” refer to the slowing, halting, or reversing the growth or progression of a disease, infection, condition, or group of cells. The inhibition can be greater than about 20%, 40%, 60%, 80%, 90%, 95%, or 99%, for example, compared to the growth or progression that occurs in the absence of the treatment or contacting.

Wherever the term “comprising” is used herein, options are contemplated wherein the terms “consisting of” or “consisting essentially of” are used instead. As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the aspect element. As used herein, “consisting essentially of” does not exclude materials or steps that do not materially affect the basic and novel characteristics of the aspect. In each instance herein any of the terms “comprising”, “consisting essentially of” and “consisting of” may be replaced with either of the other two terms. The disclosure illustratively described herein may be suitably practiced in the absence of any element or elements, limitation, or limitations not specifically disclosed herein.

As used herein, the terms “selectively binds to” or “preferentially binds to” mean that the compound, peptide or peptidomimetic, or other agent binds to the indicated molecule(s) or class of molecules with a higher affinity (e.g., at least 10-fold, in certain aspects of the invention: 100-fold) compared to a reference molecule.

As used herein, “sequence identity” or “identity” in the context of two nucleic acid or polypeptide sequences makes reference to a specified percentage of residues in the two sequences that are the same when aligned for maximum correspondence over a specified comparison window, as measured by sequence comparison algorithms or by visual inspection. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity.” Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, Calif.).

As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

The term “substantial identity” in the context of a peptide indicates that a peptide comprises a sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, or 94%, or even 95%, 96%, 97%, 98% or 99%, sequence identity to the reference sequence over a specified comparison window. In certain embodiments, optimal alignment is conducted using the homology alignment algorithm of Needleman and Wunsch (Needleman and Wunsch, JMB, 48, 443 (1970)). An indication that two peptide sequences are substantially identical is that one peptide is immunologically reactive with antibodies raised against the second peptide. Thus, a peptide is substantially identical to a second peptide, for example, where the two peptides differ only by a conservative substitution. Thus, the invention also provides nucleic acid molecules and peptides that are substantially identical to the nucleic acid molecules and peptides presented herein.

For sequence comparison, typically one sequence acts as a reference sequence to which test sequences are compared. When using a sequence comparison algorithm, test and reference sequences are input into a computer, subsequence coordinates are designated if necessary, and sequence algorithm program parameters are designated. The sequence comparison algorithm then calculates the percent sequence identity for the test sequence(s) relative to the reference sequence, based on the designated program parameters.

Nucleic acid sequences cited herein are written in a 5′ to 3′ direction unless indicated otherwise. The term “nucleic acid” refers to either DNA or RNA or a modified form thereof comprising the purine or pyrimidine bases present in DNA (adenine “A”, cytosine “C”, guanine “G”, thymine “T”) or in RNA (adenine “A”, cytosine “C”, guanine “G”, uracil “U”). Interfering RNAs provided herein may comprise “T” bases, for example at 3′ ends, even though “T” bases do not naturally occur in RNA. In some cases, these bases may appear as “dT” to differentiate deoxyribonucleotides present in a chain of ribonucleotides.

The term “polynucleotide” refers to a polymeric form of nucleotides of any length, either deoxyribonucleotides or ribonucleotides or analogs thereof. Polynucleotides can have any three-dimensional structure and may perform any function, known or unknown. The following are non-limiting examples of polynucleotides: a gene or gene fragment (for example, a probe, primer, or EST), exons, introns, messenger RNA (mRNA), transfer RNA, ribosomal RNA, ribozymes, cDNA, RNAi, siRNA, recombinant polynucleotides, branched polynucleotides, plasmids, vectors, isolated DNA of any sequence, isolated RNA of any sequence, nucleic acid probes and primers. A polynucleotide can comprise, or alternatively consist essentially of, or yet further consist of modified nucleotides, such as methylated nucleotides and nucleotide analogs. If present, modifications to the nucleotide structure can be imparted before or after assembly of the polynucleotide. The sequence of nucleotides can be interrupted by non-nucleotide components. A polynucleotide can be further modified after polymerization, such as by conjugation with a labeling component. The term also refers to both double- and single-stranded molecules. Unless otherwise specified or required, any embodiment of this invention that is a polynucleotide encompasses both the double-stranded form and each of two complementary single-stranded forms known or predicted to make up the double-stranded form.

A polynucleotide is composed of a specific sequence of four nucleotide bases: adenine (A); cytosine (C); guanine (G); thymine (T); and uracil (U) for thymine when the polynucleotide is RNA. Thus, the term “polynucleotide sequence” is the alphabetical representation of a polynucleotide molecule. This alphabetical representation can be input into databases in a computer having a central processing unit and used for bioinformatics applications such as functional genomics and homology searching.

A polynucleotide of this invention can be delivered to a cell or tissue using a gene delivery vehicle. “Gene delivery,” “gene transfer,” “transducing,” and the like as used herein, are terms referring to the introduction of an exogenous polynucleotide (sometimes referred to as a “transgene”) into a host cell, irrespective of the method used for the introduction. Such methods include a variety of well-known techniques such as vector-mediated gene transfer (by, e.g., viral infection/transfection, or various other protein-based or lipid-based gene delivery complexes) as well as techniques facilitating the delivery of “naked” polynucleotides (such as electroporation, “gene gun” delivery and various other techniques used for the introduction of polynucleotides). The introduced polynucleotide may be stably or transiently maintained in the host cell. Stable maintenance typically requires that the introduced polynucleotide either contains an origin of replication compatible with the host cell or integrates into a replicon of the host cell such as an extrachromosomal replicon (e.g., a plasmid) or a nuclear or mitochondrial chromosome. A number of vectors are known to be capable of mediating transfer of genes to mammalian cells, as is known in the art and described herein.

A “viral vector” is defined as a recombinantly produced virus or viral particle that comprises, or alternatively consists essentially of, or yet further consists of a polynucleotide to be delivered into a host cell, either in vivo, ex vivo or in vitro. Examples of viral vectors include retroviral vectors, adenovirus vectors, adeno-associated virus vectors, alphavirus vectors and the like. Alphavirus vectors, such as Semliki Forest virus-based vectors and Sindbis virus-based vectors, have also been developed for use in gene therapy and immunotherapy. See, Schlesinger and Dubensky (1999) Curr. Opin. Biotechnol. 5:434-439 and Ying, et al. (1999) Nat. Med. 5(7):823-827. In aspects where gene transfer is mediated by a retroviral vector, a vector construct refers to the polynucleotide comprising, or alternatively consisting essentially of, or yet further consisting of the retroviral genome or part thereof, and a therapeutic gene.

As used herein, “retroviral mediated gene transfer” or “retroviral transduction” carries the same meaning and refers to the process by which a gene or nucleic acid sequences are stably transferred into the host cell by virtue of the virus entering the cell and integrating its genome into the host cell genome. The virus can enter the host cell via its normal mechanism of infection or be modified such that it binds to a different host cell surface receptor or ligand to enter the cell. As used herein, retroviral vector refers to a viral particle capable of introducing exogenous nucleic acid into a cell through a viral or viral-like entry mechanism.

Retroviruses carry their genetic information in the form of RNA; however, once the virus infects a cell, the RNA is reverse-transcribed into the DNA form which integrates into the genomic DNA of the infected cell. The integrated DNA form is called a provirus.

In aspects where gene transfer is mediated by a DNA viral vector, such as an adenovirus (Ad) or adeno-associated virus (AAV), a vector construct refers to the polynucleotide comprising, or alternatively consisting essentially of, or yet further consisting of the viral genome or part thereof, and a transgene. Adenoviruses (Ads) are a relatively well characterized, homogenous group of viruses, including over 50 serotypes. See, e.g., International PCT Application No. WO 95/27071. Ads do not require integration into the host cell genome. Recombinant Ad derived vectors, particularly those that reduce the potential for recombination and generation of wild-type virus, have also been constructed. See, International PCT Application Nos. WO 95/00655 and WO 95/11984. Wild-type AAV has high infectivity and specificity integrating into the host cell's genome. See, Hermonat et al., (1984) Proc. Natl. Acad. Sci. USA 81:6466-6470 and Lebkowski et al. (1988) Mol. Cell. Biol. 8:3988-3996.

Vectors that contain both a promoter and a cloning site into which a polynucleotide can be operatively linked are well known in the art. Such vectors are capable of transcribing RNA in vitro or in vivo and are commercially available from sources such as Stratagene (La Jolla, Calif.) and Promega Biotech (Madison, Wis.). In order to optimize expression and/or in vitro transcription, it may be necessary to remove, add or alter 5′ and/or 3′ untranslated portions of the clones to eliminate extra, potential inappropriate alternative translation initiation codons or other sequences that may interfere with or reduce expression, either at the level of transcription or translation. Alternatively, consensus ribosome binding sites can be inserted immediately 5′ of the start codon to enhance expression.

A “plasmid” is an extra-chromosomal DNA molecule separate from the chromosomal DNA which is capable of replicating independently of the chromosomal DNA. In many cases, it is circular and double-stranded. Plasmids provide a mechanism for horizontal gene transfer within a population of microbes and typically provide a selective advantage under a given environmental state. Plasmids may carry genes that provide resistance to naturally occurring antibiotics in a competitive environmental niche, or alternatively the proteins produced may act as toxins under similar circumstances.

“Plasmids” used in genetic engineering are called “plasmid vectors”. Many plasmids are commercially available for such uses. The gene to be replicated is inserted into copies of a plasmid containing genes that make cells resistant to particular antibiotics and a multiple cloning site (MCS, or polylinker), which is a short region containing several commonly used restriction sites allowing the easy insertion of DNA fragments at this location. Another major use of plasmids is to make large amounts of proteins. In this case, researchers grow bacteria containing a plasmid harboring the gene of interest. Just as the bacterium produces proteins to confer its antibiotic resistance, it can also be induced to produce large amounts of proteins from the inserted gene. This is a cheap and easy way of mass-producing a gene or the protein it then codes for.

Extracellular cell-derived vesicles, also referred to as extracellular vesicles, are membrane surrounded structures that are released by cells in vitro and in vivo. Extracellular vesicles can contain proteins, lipids, and nucleic acids and can mediate intercellular communication between different cells, including different cell types, in the body. Two types of extracellular vesicles are exosomes and microvesicles. Exosomes range in size from approximately 30 nm to about 200 nm. Exosomes are released from a cell by fusion of multivesicular endosomes (MVE) with the plasma membrane. Microvesicles, on the other hand, are released from a cell upon direct budding from the plasma membrane (PM). Microvesicles are typically larger than exosomes and range from approximately 100 nm to 1 m. Also intended within this term are liposomes and apoptotic bodies.

As used herein the term “apoptotic body” intends the vesicles that are produced when a cell breaks down. Apoptotic bodies consist of cytoplasm with tightly packed organelles with or without a nuclear fragment.

As used herein, the term “fusion polypeptide” refers to proteins or polypeptides created through the joining of two or more genes that originally coded for separate polypeptides.

### Embodiments of the Invention

This disclosure provides for engineered extracellular vesicles expressing one or more fusion proteins that may be used, for example, to activate and recruit immune cells (e.g., T-cells) to kill cancer cells. Embodiments of an engineered extracellular vesicle may comprise, consist essentially of, or consist of a first fusion protein comprising a first antibody moiety, a second antibody moiety, and a transmembrane domain of an exosomal membrane protein, and a second fusion protein comprising a first protein binding moiety, an exosomal membrane protein, and a second protein binding moiety, wherein both the first fusion protein and the second fusion protein are displayed on the surface of the engineered extracellular vesicle.

In some embodiments, the engineered extracellular vesicle is an exosome or other membrane-enclosed bodies such as described in PCT Pat. Pub. Nos. WO/2017/161010, WO/2016/077639, and U.S. Pat. Pub. Nos. 20160168572, 20150290343, and 20070298118. The extracellular vesicle, e.g., a cell-derived vesicle comprising a membrane that encloses an internal space and has a smaller diameter than the cell from which it is derived may have a diameter from 20 nm to 1000 nm. In some embodiments, the engineered extracellular vesicle comprises an apoptotic body, a fragment of a cell, a vesicle derived from a cell by direct or indirect manipulation, a vesiculated organelle, and a vesicle produced by a living cell (e.g., by direct plasma membrane budding or fusion of the late endosome with the plasma membrane), or an exosome.

In embodiments, the engineered extracellular vesicle comprises an exosome. In some embodiments, the exosome is a cell-derived small (e.g., between 20-300 nm in diameter, or 40-200 nm in diameter) vesicle comprising a membrane that encloses an internal space, and which is generated from said cell by direct plasma membrane budding or by fusion of the late endosome with the plasma membrane. In embodiments, production of exosomes does not result in the destruction of the source cell. In embodiments, the exosome comprises lipid or fatty acid and polypeptide.

Populations of engineered extracellular vesicles (e.g., exosomes and/or microvesicles) of the present disclosure can be isolated using any method known by those in the art. Non-limiting examples include differential centrifugation by ultracentrifugation (Thery et al. (2006) Curr. Protoc. Cell Biol. 30:3.22.1-3.22.29; Witmer et al. (2013) J. Extracellular v.2), sucrose gradient purification (Escola et al. (1998) J. Biol. Chem. 273:20121-20127), and combination filtration/concentration (Lamparski et al. (2002) J. Immunol. Methods 270:211-226).

After isolation, the cell-derived vesicles, e.g., exosomes can be concentrated to provide a purified population of cell-derived vesicles. Any appropriate method can be used to concentrate the cell-derived vesicles, e.g., exosomes. Non-limiting examples of such include centrifugation, ultrafiltration, filtration, differential centrifugation and column filtration. Further sub-populations can be isolated using antibodies or other agents that are specific for a specific marker expressed by the desired exosome population. Alternatively, shed exosomes may be purified using commercially available extraction kits such as ExoQuick™ and Total Exosome Isolation™.

The engineered extracellular vesicles can be made from several different types of lipids, e.g., amphipathic lipids, such as phospholipids. The engineered extracellular vesicle may comprise a lipid bilayer as the outermost surface. This bilayer may be comprised of one or more lipids of the same or different type. Examples include, but are not limited to, phospholipids such as phosphocholines and phosphoinositols. Specific examples include without limitation, and DSPC, DOPC, and DMPC,

An engineered extracellular vesicle may be mainly comprised of natural phospholipids and lipids such as 1,2-distearoryl-sn-glycero-3-phosphatidyl choline (DSPC), sphingomyelin, egg phosphatidylcholines and monosialoganglioside. In embodiments, a engineered extracellular vesicle comprises only phospholipids and is less stable in plasma. However, manipulation of the lipid membrane with cholesterol can, in embodiments, increase stability and reduce rapid release of the encapsulated bioactive compound into the plasma. In some embodiments, phospholipid may be phosphatidylcholine 1,2-dioleoyl-sn-glycero-3-phosphocholine is abbreviated herein as “DOPC”. In some embodiments, the engineered extracellular vesicle comprises 1,2-dioleoyl-sn-glycero-3-phosphoethanolamine (DOPE), e.g., to increase stability (see, e.g., Spuch and Navarro, Journal of Drug Delivery, vol. 2011, Article ID 469679, 12 pages, 2011. doi:10.1155/2011/469679 for review). In other embodiments, an engineered extracellular vesicle may comprise the phospholipid 1,2-dimyristoyl-sn-glycero-3-phosphocholine (dimyristoylphosphocholine; DMPC).

In some embodiments, engineered extracellular vesicles comprise or are enriched for lipids that affect membrane curvature (see, e.g., Thiam et al., Nature Reviews Molecular Cell Biology, 14(12): 775-785, 2013). Some lipids have a small hydrophilic head group and large hydrophobic tails, which facilitate the formation of a fusion pore by concentrating in a local region. In some embodiments, engineered extracellular vesicles comprise or are enriched for negative-curvature lipids, such as cholesterol, phosphatidylethanolamine (PE), diglyceride (DAG), phosphatidic acid (PA), fatty acid (FA). In some embodiments, engineered extracellular vesicles do not comprise, are depleted of, or have few positive-curvature lipids, such as lysophosphatidylcholine (LPC), phosphatidylinositol (Ptdlns), lysophosphatidic acid (LPA), lysophosphatidylethanolamine (LPE), monoacylglycerol (MAG).

In some embodiments, the lipids are added to a source cell to produce an engineered extracelluar vesicle. In some embodiments, the lipids are added to source cells in culture which incorporate the lipids into their membranes prior to or during the formation of an engineered extracellular vesicle. In some embodiments, the lipids are added to the cells or engineered extracellular vesicle in the form of a liposome. In some embodiments, methyl-betacyclodextrane (mo-CD) is used to enrich or deplete lipids (see, e.g., Kainu et al, Journal of Lipid Research, 51(12): 3533-3541, 2010).

In this way, the engineered extracellular vesicles may comprise, for example, DOPE (dioleoylphosphatidylethanolamine), DOTMA, DOTAP, DOTIM, DDAB, alone or together with cholesterol to yield DOPE and cholesterol, DOTMA and cholesterol, DOTAP and cholesterol, DOTIM and cholesterol, and DDAB and cholesterol. Methods for preparation of multilamellar vesicle lipids are known in the art (see for example U.S. Pat. No. 6,693,086, the teachings of which relating to multilamellar vesicle lipid preparation are incorporated herein by reference). Although formation of engineered extracellular vesicles can be spontaneous when a lipid film is mixed with an aqueous solution, it can also be expedited by applying force in the form of shaking by using a homogenizer, sonicator, or an extrusion apparatus (see, e.g., Spuch and Navarro, Journal of Drug Delivery, vol. 2011, Article ID 469679, 12 pages, 2011. doi:10.1155/2011/469679 for review). Extruded lipids can be prepared by extruding through filters of decreasing size, as described in Templeton et al., Nature Biotech, 15:647-652, 1997, the teachings of which relating to extruded lipid preparation are incorporated herein by reference.

In another embodiment of the engineered extracellular vesicles, the lipids may include, but are not limited to, DLin-KC2-DMA4, C12-200 and co-lipids disteroylphosphatidyl choline, cholesterol, and PEG-DMG may be formulated (see, e.g., Novobrantseva, Molecular Therapy-Nucleic Acids (2012) 1, e4; doi:10.1038/mtna.2011.3) using a spontaneous vesicle formation procedure. Tekmira publications describe various aspects of lipid vesicles and lipid vesicle formulations (see, e.g., U.S. Pat. Nos. 7,982,027; 7,799,565; 8,058,069; 8,283,333; 7,901,708; 7,745,651; 7,803,397; 8,101,741; 8,188,263; 7,915,399; and 8,236,943, all of which are herein incorporated by reference and may be used and/or adapted to the present invention.

In some embodiments, an engineered extracellular vesicle described herein may include one or more polymers. The polymers may be biodegradable. Biodegradable polymer vesicles may be synthesized using methods known in the art. Exemplary methods for synthesizing polymer vesicles are described by Bershteyn et al., Soft Matter 4:1787-1787, 2008 and in U.S. Pat. Pub. No. 2008/0014144 A1, the specific teachings of which relating to microparticle synthesis are incorporated herein by reference.

Exemplary synthetic polymers which can be used include without limitation aliphatic polyesters, polyethylene glycol (PEG), poly (lactic acid) (PLA), poly (glycolic acid) (PGA), co-polymers of lactic acid and glycolic acid (PLGA), polycarprolactone (PCL), polyanhydrides, poly(ortho)esters, polyurethanes, poly(butyric acid), poly(valeric acid), and poly(lactide-co-caprolactone), and natural polymers such as albumin, alginate and other polysaccharides including dextran and cellulose, collagen, chemical derivatives thereof, including substitutions, additions of chemical groups such as for example alkyl, alkylene, hydroxylations, oxidations, and other modifications routinely made by those skilled in the art), albumin and other hydrophilic proteins, zein and other prolamines and hydrophobic proteins, copolymers and mixtures thereof. In general, these materials degrade either by enzymatic hydrolysis or exposure to water in vivo, by surface or bulk erosion.

In some embodiments the engineered extracellular vesicle comprises one or more of an exosome, a liposome, a microvesicle, and an apoptotic body. In some embodiments, the engineered extracellular vesicle is an exosome having an average particle size of about 25 nm to about 200 nm., or about 30 nm to about 150 nm, or about 30 nm to about 100 nm.

In some embodiments of an engineered extracellular vesicle, a first fusion protein comprises a formula A-B-C, wherein A is a first antibody moiety, B is a second antibody, and C is an exosomal membrane protein transmembrane domain moiety, wherein the first fusion is displayed on a surface of the engineered extracellular vesicle. In other embodiments of an engineered extracellular vesicle, a first fusion protein comprises a formula A-L₁-B-L₂-C, wherein A is a first antibody moiety, L₁ is a first linker; B is a second antibody, L₂ is a second linker, and C is an exosomal membrane protein transmembrane domain moiety, wherein the first fusion is displayed on a surface of the engineered extracellular vesicle.

In some embodiments of the engineered extracellular vesicle, the first antibody moiety and the second antibody moiety are a single chain variable fragment (scFv), a single domain antibody, a bispecific antibody, or a multispecific antibody.

The term “antibody” as used herein refers to a polypeptide (or set of polyptptides) of the immunoglobulin family that is capable of binding an antigen non-covalently, reversibly and specifically. For example, a naturally occurring “antibody” of the IgG type is a tetramer comprising at least two heavy (H) chains and two light (L) chains inter-connected by disulfide bonds. Each heavy chain is comprised of a heavy chain variable region (abbreviated herein as V_{H}) and a heavy chain constant region. The heavy chain constant region is comprised of three domains, CH1, CH2 and CH3. Each light chain is comprised of a light chain variable region (abbreviated herein as V_{L}) and a light chain constant region. The light chain constant region is comprised of one domain, CL. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs arranged from amino-terminus to carboxy-terminus in the following order: FR1, CDR1, FR2, CDR2, FR3, CDR3, FR4. The variable regions of the heavy and light chains contain a binding domain that interacts with an antigen, which is sometimes referred to herein as the antigen binding domain. The constant regions of the antibodies may mediate the binding of the immunoglobulin to host tissues or factors, including various cells of the immune system (e.g., effector cells) and the first component (Clq) of the classical complement system. The term “antibody” includes, but is not limited to, monoclonal antibodies, human antibodies, humanized antibodies, camelised antibodies, chimeric antibodies, bispecific or multispecific antibodies and anti-idiotypic (anti-Id) antibodies (including, e.g., anti-Id antibodies to antibodies described herein), single chain variable fragments, and single domain antibodies. The antibodies can be of any isotype/class (e.g., IgG, IgE, IgM, IgD, IgA and IgY) or subclass (e.g., IgG1, IgG2, IgG3, IgG4, IgA1 and IgA2). Both the light and heavy chains are divided into regions of structural and functional homology. The terms “constant” and “variable” are used functionally. In this regard, it will be appreciated that the variable domains of both the light (V_{L}) and heavy (V_{H}) chain portions determine antigen recognition and specificity. Conversely, the constant domains of the light chain (CL) and the heavy chain (CH1, CH2 or CH3) confer important biological properties such as secretion, transplacental mobility, Fc receptor binding, complement binding, and the like. By convention the numbering of the constant region domains increases as they become more distal from the antigen binding site or amino-terminus of the antibody. The N-terminus is a variable region and at the C-terminus is a constant region; the CH3 and CL domains actually comprise the carboxy-terminus of the heavy and light chain, respectively. Examples of antibodies and methods of preparing the same are described, for example, in U.S. Pat. Nos. 4,816,567; 5,789,215; 6,596,541; 7,582,298; and 8,502,018.

Preferably, both the first antibody moiety and the second antibody moiety are scFvs. The term “scFv” refers to a fusion protein comprising at least one antibody fragment comprising a variable region of a light chain and at least one antibody fragment comprising a variable region of a heavy chain, wherein the light and heavy chain variable regions are contiguously linked via a short flexible polypeptide linker, and capable of being expressed as a single chain polypeptide, and wherein the scFv retains the specificity of the intact antibody from which it is derived. Unless specified, as used herein an scFv may have the V_{L} and V_{H} variable regions in either order, e.g., with respect to the N-terminal and C-terminal ends of the polypeptide, the scFv may comprise V_{L}-linker-V_{H} or may comprise V_{H}-linker-V_{L}. ScFv molecules are known in the art and their production is described, for example, in U.S. Pat. Nos. 4,946,778 and 5,641,870.

In other embodiments, the first antibody moiety and/or the second antibody moiety are bispecific antibodies. The term “bispecific antibody” refers to an antibody that shows specificities to two different types of antigens. In other embodiments, the first antibody binding moiety and/or the second antibody binding moiety are multispecific antibodies. The term “multispecific antibody” as used herein refers to a molecule that binds to two or more different epitopes on one antigen or on two or more different antigens. Recognition of each antigen is generally accomplished with an “antigen binding domain”. The multispecific antibody may include one polypeptide chain that comprises a plurality, e.g., two or more, e.g., two, antigen binding domains. In some embodiments, the multispecific antibody may include two, three, four or more polypeptide chains that together comprise a plurality, e.g., two or more, e.g., two, antigen binding domains. Examples of the production and isolation of bispecific and multispecific antibodies are described in, for example, PCT Pat. Pubs. WO2014031174 and WO2009080252.

Also contemplated are first and second antibody moieties comprising single domain antibodies. The term “single domain antibodies” refers to the variable regions of either the heavy (V_{H}) or light (VL) chain of an antibody. Single domain antibodies are described, for example in U.S. Pat. Pub. No. 20060002935.

In some embodiments, the first and second antibody moieties are anchored to and displayed on the surface of an extracellular vesicle such as an exosome. In some embodiments, the first a second antibody moieties are fused to a portion of an exosomal membrane protein. In some embodiments, the portion of an exosomal membrane protein is a transmembrane domain of plate derived growth factor receptor (PDGFR) (SEQ ID NO: 5).

In certain embodiments, the scFv molecules may be produced from cDNA molecules or other polynucleotides encoding the variable regions of the heavy and light chains of the mAb that may be amplified by standard polymerase chain reaction (PCR) methodology using a set of primers for immunoglobulin heavy and light variable regions (Clackson (1991) Nature, 352, 624-628) (Also see U.S. Pat. No. 6,287,569). The amplified cDNAs encoding mAb heavy and light chain variable regions then may be linked together with a linker polypeptide in order to generate a recombinant scFv DNA molecule. Other polynucleotide elements maybe included in the recombinant fusion protein such as an epitope tag and/or another protein that may anchor the scFv molecule on the surface of the exosome. In certain embodiments, the scFv molecules are genetically fused to the polynucleotide sequence of, for example, one or more of a hemagglutinin epitope tag, a 6× histidine tag (HHHHHH) (SEQ ID NO: 22), and a transmembrane segment of PDGFR.

In some embodiments, the first and second antibody moieties bind separately to an immune cell (e.g., T-cell) marker protein comprising one or more of CD3 (CD3d:Entrez gene: 915; RefSeq: NP_000723.1, NP_001035741.1; CD3e: Entrez gene: 916; RefSeq: NP_000724.1; CD3g: Entrez gene: 917; RefSeq: NP_000064.1), CD2 (Entrez gene: 914; RefSeq: NP_001315538.1, NP_001758.2), CD4 (Entrez gene: 920; RefSeq: NP_000607.1, NP_001181943.1, NP_001181944.1, NP_001181945.1, NP_001181946.1, NP_001369634.1, NP_001369635.1, NP_001369636.1, NP_001369643.1), CD5 (Entrez gene: 921; RefSeq: NP_001333385.1, NP_055022.2), CD7 (Entrez gene: 924; RefSeq: NP_006128.1), CD8 (CD8a Entrez gene: 925; RefSeq: NP_001139345.1, NP_001759.3, NP_001369627.1, NP_741969.1; CD8b: Entrez gene: 926; RefSeq: NP_001171571.1, NP_004922.1, NP_742099.1, NP_742100.1, NP_757362.1), CD14 (Entrez gene: 929; RefSeq: NP_000582.1, NP_001035110.1, NP_001167575.1, NP_001167576.1), CD15 (Entrez gene: 2526; RefSeq: NP_002024.1), CD16 (CD16a: Entrez gene 2214; RefSeq: NP_000560.7, NP_001121064.2, NP_001121065.1, NP_001121067.1, NP_001121068.1, NP_001316049.1, NP_001316051.1, NP_001373379.1; CD16b: Entrez gene: 2215; RefSeq: NP_000561.3, NP_001231682.1, NP_001257964.1, NP_001257965.1, NP_001257966.1), CD24 (Entrez gene: 100133941; RefSeq: NP_001278666.1, NP_001278667.1, NP_00001278668.1, NP_001346013.1, NP_037362.1), CD25 (Entrez gene: 3559; RefSeq: NP_000408.1, NP_001295171.1, NP_001295172.1), CD27 (Entrez gene: 939; RefSeq: NP_001233.1), CD28 (Entrez gene: 940; RefSeq: NP_001230006.1, NP_001230007, NP_006130.1), CD30 (Entrez gene: 943; RefSeq: NP_001234.3, NP_001268359.2), CD31 (Entrez gene: 5175; RefSeq: NP_0000433.4), CD38 (Entrez gene: 952; RefSeq: NP_001766.2), CD40L (Entrez gene: 959; RefSeq: NP_000065.1), CD45 (Entrez gene: 5788; RefSeq: NP_001254727.1, NP_002829.3, NP_563578.2), CD56 (Entrez gene: 46841; RefSeq: NP_000606.3, NP_001070150.1, NP_001229536.1, NP_001229537.1 NP_001373218.1, NP_001373219.1, NP_001373220.1, NP_001373221.1), CD68 (Entrez gene: 968; RefSeq: NP_001035148.1, NP_001242.2), CD91 (Entrez gene: 4035; RefSeq: NP_002323.2), CD114 (Entrez gene: 1441; RefSeq: NP_000751.1, NP_724781.1, NP_758519.1), CD163 (Entrez gene: 9332; RefSeq: NP_001357074.1, NP_001357075.1, NP_004235.4, NP_981961.2, CD206 (Entrez gene: 4360; RefSeq: NP_002429.1), LFA1 (Entrez gene: 3689; RefSeq: NP_000202.3, NP_001120963.2, NP_001290167.1), PD-1 (Entrez gene: 5133; RefSeq: NP_005009.2), ICOS (Entrez gene: 29851; RefSeq: NP_036224.1), BTLA (Entrez gene: 151888; RefSeq: NP_001078826.1, NP_861445.4), KIR (Entrez gene: 3811; RefSeq: NP_001309097.1, NP_037421.2), CD137 (Entrez gene: 3604; RefSeq: NP_001552.2), OX40 (Entrez gene: 7293; RefSeq: NP_003318.1), LAG3 (Entrez gene: 3902; RefSeq: NP_002277.4), CTLA4 (Entrez gene: 1493; RefSeq: NP_001032720.1, NP_005205.2), T-cell Receptor (Entrez gene: 6955; UniProtKB/Swiss-Prot: P0DSE1,P0DTU3), Epidermal Growth Factor Receptor (Entrez gene: 1956; RefSeq: NP_001333826.1, NP_001333827.1, NP_001333828.1, NP_001333829.1, NP_001333870.1, NP_005219.2, NP_958439.1, NP_958440.1, NP_958441.1), CLL-1 (Entrez gene: 160364; RefSeq: NP_001193939.1, NP_001287659.1, NP_612210.4, NP_963917.2), HER2, (Entrez gene: 2064; RefSeq: NP_001005862.1, NP_001276865.1, NP_001276866.1), HER3 (Entrez gene: 2065; RefSeq: NP_001005915.1, NP_001973.2), CD33 (Entrez gene: 945; RefSeq: NP_001076087.1, NP_001171079.1, NP_001763.3), CD34, (Entrez gene: 947; RefSeq: NP_001020280.1, NP_001764.1), CD38, (Entrez gene: 952; RefSeq: NP_001766.2), CD123 (Entrez gene: 3563; RefSeq: NP_001254642.1, NP_002174.1), TIM3 (Entrez gene: 84868; RefSeq: NP_116171.3), CD25 (Entrez gene: 3559; RefSeq: NP_000408.1, NP_001295171.1, NP_001295172.1), CD32 (Entrez gene: 2212; RefSeq: NP_001129691.1, NP_001362225.1, NP_001362226.1, NP_067674.2), CD96 (Entrez gene: 10225; RefSeq: NP_001305818.1, NP_005807.1, NP_937839.1), and protein death ligand 1 or 2 (PD-L1/L2) (Entrez gene: 29126; RefSeq: NP_001254635.1, NP_001300958.1, NP_054862.1). The Entrez gene numbers and RefSeq numbers may be accessed through the NCBI home page or www.ncbi.nlm.nih.gov.

Non-limiting examples of immune cells are selected from the group consisting of: a CD3⁺ T cell, a CD16⁺ cell, a CD16⁺ NK cell, a CD4 cell, a CD8 cell, a CD19 cell, a CD20 cell, or a B cell. In preferred embodiments, the immune cell is a T-Cell.

Accordingly, in some embodiments, one of the first and second antibody moieties is an scFv fragments such as, for example, anti-CD3, anti-OX40, anti-CD2, anti-CD4, anti-CD5, anti-CD7, anti-CD8, anti-CD14, anti-CD15, anti-CD16, anti-CD24, anti-CD25, anti-CD27, anti-CD28, anti-CD30, anti-CD31, anti-CD38, anti-CD40L, anti-CD45, anti-CD56, anti-CD68, anti-CD91, anti-CD114, anti-CD163, anti-CD206, anti-LFA1, anti-PD-1, anti-ICOS, anti-BTLA, anti-KIR, anti-CD137, anti-LAG3, anti-CTLA4, or anti-T-cell Receptor; and one of the first and second antibody moieties is an scFv fragments such, for example, anti-EGFR, anti-CLL-1, anti-HER2, anti-HER3, anti-CD33, anti-CD34, anti-CD38, anti-CD123, anti-TIM3, anti-CD25, anti-CD32, anti-CD96, anti-PD-L1, or anti-PD-L2.

In some embodiments, the first antibody moiety binds to immune cell marker protein and the second antibody binds to a cancer cell surface marker protein, or vice versa. Preferably, the first and second antibody moieties are not the same and selectively bind to different targets.

In some embodiments, the cancer cell surface-marker protein is epidermal growth factor receptor (EGFR) and the immune cell marker protein is CD3.

In Some embodiments, the second fusion protein comprises, consists essentially of, or consist of the formula D-E-F, wherein D is a first protein binding moiety, E is a second exosomal membrane protein transmembrane domain, and F is a second protein binding moiety. In other embodiments, the second fusion protein comprises, consists essentially of, or consists of the formula D-L₃-E-L₄-F, wherein D is a first protein binding moiety, L₃ is a third linker, E is an exosomal protein transmembrane domain; L₄ is a fourth linker, and F is a second protein binding moiety, wherein the second fusion is displayed on a surface of the engineered extracellular vesicle.

In some embodiments, the first protein binding moiety is a type I membrane protein and the second protein binding moiety is a type II membrane protein. As used herein, type I membrane proteins include a single transmembrane domain and a cytoplasmic C-terminus and an extracellular or luminal N-terminus for plasma membrane or organelle membrane, respectively. Type II membrane proteins have the opposite C and N-terminal orientation compared to type I proteins.

Preferably, the first and second protein binding moieties are not identical proteins, and/or bind to different proteins. In some embodiments, the type I membrane protein is PD-1, and the type II membrane protein is OX40L, wherein the PD-1 binds to PD-L1/L2, and the OX40L binds to OX40, and wherein the PD-L1/L2 and the OX40 are disposed on a surface of a tumor cell and an immune cell, respectively.

In other embodiments, the type I membrane protein is LAG3 (Entrez gene: 3902; RefSeq: NP_002277.4), TIM-3 (Entrez gene: 84868; RefSeq: NP_116171.3), KIR (Entrez gene: 3811; RefSeq: NP_001309097.1), CD96 (Entrez gene: 10225; RefSeq: NP_001305818.1), CTLA-4 (Entrez gene: 1493; RefSeq: NP_001032720.1), BTLA (Entrez gene: 151888; RefSeq: NP_001078826.1), SIRPa (Entrez gene: 140885; RefSeq: NP_001035111.1), or CD200 (Entrez gene: 4345; RefSeq: NP_001004196.2), wherein a complementary binding target of the first protein binding moiety is disposed on a surface of a tumor cell.

In yet further embodiments, the type II membrane protein is 4-1BBL (Entrez gene: 8744; RefSeq: NP_003802.1), CD70 (Entrez gene: 970; RefSeq: NP_001243.1), GITRL (Entrez gene: 8995; RefSeq: NP_005083.3), CD40L (Entrez gene: 959; RefSeq: NP_000065.1), CD30L (Entrez gene: 944; RefSeq: NP_001235.1), or TL1A (Entrez gene: 9966; RefSeq: NP_001191273.1), wherein a complementary binding target of the second protein binding moiety is disposed on a surface of an immune cell. As used herein, the term “complementary binding target” refers to a natural ligand of the subject protein (e.g., receptor-ligand interaction). The binding partners of the listed protein binding moieties are known in the art and can be found using the Entrez/RefSeq numbers listed herein.

In other embodiments, the first protein binding moiety is a type II membrane protein as described herein and the second protein binding moiety is a type I membrane protein as described herein.

In some embodiments, the first antibody moiety is an scFv binds to an immune cell marker protein, wherein the immune cell marker protein is CD3; and the second antibody moiety is an scFv binds to a cancer cell surface-marker protein, wherein the cell surface marker protein is epidermal growth factor receptor (EGFR); or the first antibody moiety is an scFv binds to a cancer cell surface-marker protein, wherein the cell surface marker protein is epidermal growth factor receptor (EGFR); and the second antibody moiety is an scFv binds to an immune cell marker protein, wherein the immune cell marker protein is CD3.

In some embodiments, the first protein binding moiety is PD-1, and the second protein binding moiety is OX40L; or the first protein binding moiety is OX40L, and the second protein binding moiety is PD-1.

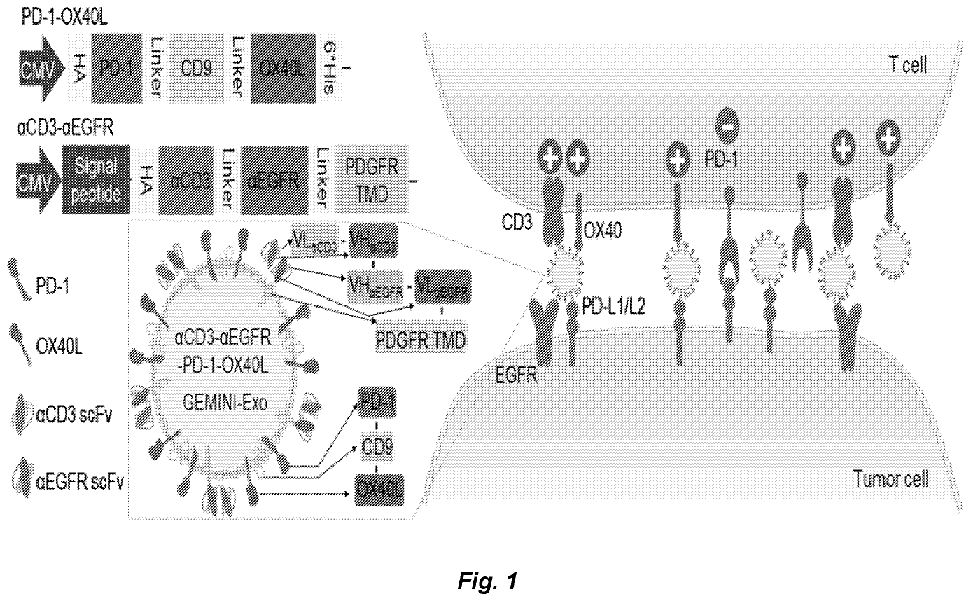

In some embodiments, the exosomal membrane protein of the second fusion protein is CD9 (Entrez gene: 928; RefSeq: NM_001769, NP_001317241, NP_001760) or portions thereof, such as a portion of CD9 comprising a transmembrane domain. Preferably, the exosomal membrane protein is positioned between the first protein binding moiety and the second protein binding moiety, such as is disclosed in FIG. 1. Alternatively, certain embodiments may include ethe use of transmembrane domain of platelet-derived growth factor receptor (PDGFR), Lam2b, lactadherin C1C2 domain, or CD13.

Peptide linker groups may be used to connect various portions of the fusion proteins, for example, between an antibody moiety (e.g., scFv) and the PDGFR transmembrane domain or between variable heavy and variable light chain of the antibody moiety. In other embodiments, additional linker proteins may be positioned between the exosomal transmembrane proteins (e.g., CD9) and each of the flanking protein binding moieties. For example, in some embodiments, flexible (GGGGS)₂ linkers (SEQ ID NO: 33) are disposed between the CD9 protein and each of the flanking protein binding moieties. In other embodiments, the linker sequence is either (GGGS)n (SEQ ID NO: 34) where n is an integer between 1 and 5 or (GGGGS)n (SEQ ID NO: 36) where n is an integer between 1 and 5. In some embodiments, the(GGGS)n linker sequence (SEQ ID NO: 34) is a (GGGS)₄ peptide (SEQ ID NO: 35) and the (GGGGS)n linker sequence (SEQ ID NO: 36) is a (GGGGS)₃ peptide (SEQ ID NO: 37). The linker sequence may be varied depending on the polypeptide portions to be linked to form the fusion protein. Further linker examples include poly(L-Gly), (Poly L-Glycine linkers); poly(L-Glu), (PolyL-Glutamine linkers); poly (1-Lys), (Poly L-Lysine linkers).

Each fusion protein also may include one or more epitope tags, affinity tags, solubility enhancing tags, and the like. Examples of various additional tags and linkers that may be used with the present invention include, haemagglutinin (HA) epitope, myc epitope, histidine tag, chitin binding protein (CBP), maltose binding protein (MBP), glutathione-S-transferase (GST), calmodulin binding peptide, biotin carboxyl carrier protein (BCCP), FLAG octapeptide, nus, green fluorescent protein (GFP), thioredoxin, poly(NANP), V5, S-protein, streptavidin, SBP, poly(Arg), DsbA, c-myc-tag, HAT, cellulose binding domain, softag 1, softag3, small ubiquitin-like modifier (SUMO), and ubiquitin (Lib). In certain embodiments, the fusion protein includes an epitope tag at the n-terminus or the c-terminus of the fusion protein. In preferred embodiments, the epitope tag is a hemagglutinin (HA) epitope tag YPYDVPDYA (SEQ ID NO: 40) disposed at the N-terminus of the fusion protein, and/or a 6× Histidine tag at the C-terminal end of the fusion protein.

In some embodiments, the engineered extracellular vesicle comprises a first fusion protein comprising, consisting essentially of, or consisting of the formula T₁-A-L₁-B-L₂-C-T₂, wherein T₁ is a first epitope tag, A is the first antibody moiety, L₁ is a first linker moiety, B is the second antibody moiety, L₂ is a second linker moiety, C is the first exosomal protein transmembrane domain, and T₂ is a second epitope tag; and the second fusion protein comprises, consists essentially of, or consists of a formula T₃-D-L3-E-L4-F, wherein T3 is a third epitope tag, D is the first protein binding moiety, L₃ is a third linker moiety, E is the second exosomal membrane protein transmembrane domain, L₄ is a fourth linker moiety, and F is the second protein binding moiety.

In some embodiments, a fusion protein also may include a signal peptide (e.g., METDTLLLWVLLLWVPGSTGD; SEQ ID NO: 47) on the N-terminus of the fusion protein. For example, the first fusion protein may have the formula S₁-T₁-A-L₁-B-L₂-C-T₂, wherein S₁ is a signal peptide, T₁ is a first epitope tag, A is the first antibody moiety, L₁ is a first linker moiety, B is the second antibody moiety, L₂ is a second linker moiety, C is the first exosomal protein transmembrane domain, and T₂ is a second epitope tag.

In some embodiments, one of the first and second antibody moieties comprise an anti-CD3 variable light chain-Linker—anti-CD3 variable heavy chain (SEQ ID NO: 4) and one of the first and second antibody moieties comprise an anti-EGFR variable heavy chain-Linker—anti-EGFR variable light chain (SEQ ID NO: 5).

In some embodiments, the first fusion protein has an amino acid sequence that is about 90% identical, about 95% identical, about 96% identical, about 97% identical, about 98% identical, or about 99% identical to SEQ ID NO: 1 and the second fusion protein has an amino acid sequence that is about 90% identical, about 95% identical, about 96% identical, about 97% identical, about 98% identical, or about 99% identical to SEQ ID NO: 3. In one embodiment, the first fusion protein has an amino acid sequence that is SEQ ID NO: 1 and the second fusion protein has an amino acid sequence SEQ ID NO: 3.

In another embodiment, the first fusion protein and second fusion protein has a DNA sequence about 95% identical, about 96% identical, about 97% identical, about 98% identical, about 99% identical or identical to SEQ ID NO: 2 and SEQ ID NO: 4, respectively.

In certain embodiments, the subject fusion proteins may be delivered via an expression construct to cells, including a nucleic acid that provides a coding sequence for a fusion protein. For instance, the expression construct can encode a fusion protein that is secreted in an exosome by the transduced cell. General laboratory techniques (DNA extraction, RNA extraction, cloning, cell culturing. etc.) are known in the art and described, for example, in Molecular Cloning: A Laboratory Manual, J. Sambrook et al., 4th edition, Cold Spring Harbor Laboratory Press, 2012.

As used herein, the term “nucleic acid” refers to polynucleotides such as deoxyribonucleic acid (DNA), and, where appropriate, ribonucleic acid (RNA). The term should also be understood to include, as appropriate to the context or as applicable to the embodiment being described, both single-stranded polynucleotides (such as antisense) and double-stranded polynucleotides (such as siRNAs).

A “protein coding sequence” or a sequence that “encodes” a particular polypeptide or peptide, is a nucleic acid sequence that is transcribed (in the ease of DNA) and is translated (in the case of mRNA) into a polypeptide in vitro or in vivo when placed under the control of appropriate regulatory sequences. The boundaries of the coding sequence are determined by a start codon at the 5′ (amino) terminus and a translation stop codon at the 3′ (carboxyl) terminus. A coding sequence can include, but is not limited to, cDNA from prokaryotic or eukaryotic mRNA, genomic DNA sequences from prokaryotic or eukaryotic DNA, and even synthetic DNA sequences. A transcription termination sequence will usually be located 3′ to the coding sequence.

As used herein, the term “vector” refers to a nucleic acid molecule capable of transporting another nucleic acid to which it has been linked. One type of vector is a genomic integrated vector, or “integrated vector,” which can become integrated into the chromosomal DNA of the host cell. Another type of vector is an episomal vector, e.g., a nucleic acid capable of extra-chromosomal replication. Vectors capable of directing the expression of genes to which they are operatively linked are referred to herein as “expression vectors.” In the present specification, “plasmid” and “vector” are used interchangeably unless otherwise clear from the context. In the expression vectors, regulatory elements controlling transcription can be generally derived from mammalian, microbial, viral or insect genes. The ability to replicate in a host, usually conferred by an origin of replication, and a selection gene to facilitate recognition of transformants may additionally be incorporated. Vectors derived from viruses, such as retroviruses, adenoviruses, and the like, may be employed.

Vectors suitable for use in preparation of fusion proteins include those selected from baculovirus, phage, plasmid, phagemid, cosmid, fosmid, bacterial artificial chromosome, viral DNA, P1-based artificial chromosome, yeast plasmid, and yeast artificial chromosome. For example, the viral DNA vector can be selected from vaccinia, adenovirus, foul pox virus, pseudorabies and a derivative of SV40. Suitable bacterial vectors for use in various methods include pQE70™, pQE60, pQE-9, pBLUESCRIPT SK, pBLUESCRIPT™ KS, pTRC99a™ pKK223-3™, pDRS40™, PAC™ and pRIT2T™. Suitable eukaryotic vectors for use in various methods include pWLNEO™, pXTI™, pSG5™, pSVK3™, pBPV™, pMSG™, and pSVLSV40™. Suitable eukaryotic vectors for use in various methods include pWLNEO™ pXTI™, pSG5™, pSVK3™, pBPV™, pMSG™, and pSVLSV40™.

Polynucleotides encoding the fusion proteins can be operatively linked to regulatory elements to drive expression of the polynucleotide and can be further contained within a vector, e.g. a plasmid or a viral vector. Host cells, prokaryotic and eukaryotic cells, containing the polynucleotides and/or polypeptides and methods of expressing the polynucleotides are further provided herein, as well as the polypeptides encoded by the polynucleotides. The polynucleotides and polypeptides can further comprise, or alternatively consist essentially of, or yet further consist of a detectable and/or a purification label. The polynucleotides are useful to prepare the vesicles and for recombinant production of the fusion polypeptides by transducing a cell with the polynucleotide contained within an expression vector and culturing the cell under conditions that promote expression of the polynucleotide. The vesicles can be further isolated from the culture media.

Those of skill in the art can select a suitable regulatory region to be included in such a vector, for example from lacI, lacZ, T3, I7, apt, lambda PR, PL, trp, CMV immediate early, HSV thymidine kinase, early and late SV40, retroviral LTR, and mouse metallothionein-I regulatory regions.

Host cells in which the vectors containing the polynucleotides encoding the protein conjugates can be expressed include, for example, a bacterial cell, a eukaryotic cell, a yeast cell, an insect cell, or a plant cell. For example, E. coli, Bacillus, Streptomyces, Pichia pastoris, Salmonella typhimurium, Drosophila S2, Spodoptera SJ9, CHO, COS (e.g. COS-7), Expi293F cells, HeLa cells, HEK293T, MDA-MB-231, immature dendritic cells, stem cells, or Bowes melanoma cells are all suitable host cells for use in the methods described herein.

In some embodiments, the engineered extracellular vesicles are formulated into a composition comprising a pharmaceutically acceptable carrier or diluent. In some embodiments, a composition may include two or more engineered extracellular vesicles, each with a different complement of fusion proteins.

In some embodiments, the disclosure also provides for a method of treating cancer comprising administering an effective amount of a composition comprising one or more extracellular vesicles as disclosed herein to a subject having or suspected of having cancer whereby the engineered extracellular vesicles selectively binds to activate immune cells (e.g., T-cells) to kill the cancer cells.

The term “cancer,” as used herein, refers to any benign or malignant abnormal growth of cells. Examples include, without limitation, breast cancer, prostate cancer, lymphoma, skin cancer, pancreatic cancer, colon cancer, melanoma, malignant melanoma, ovarian cancer, brain cancer, primary brain carcinoma, head-neck cancer, glioma, glioblastoma, liver cancer, bladder cancer, non-small cell lung cancer, head or neck carcinoma, breast carcinoma, ovarian carcinoma, lung carcinoma, small-cell lung carcinoma, Wilms' tumor, cervical carcinoma, testicular carcinoma, bladder carcinoma, pancreatic carcinoma, stomach carcinoma, colon carcinoma, prostatic carcinoma, genitourinary carcinoma, thyroid carcinoma, esophageal carcinoma, myeloma, multiple myeloma, adrenal carcinoma, renal cell carcinoma, endometrial carcinoma, adrenal cortex carcinoma, malignant pancreatic insulinoma, malignant carcinoid carcinoma, choriocarcinoma, mycosis fungoides, malignant hypercalcemia, cervical hyperplasia, leukemia, acute lymphocytic leukemia, chronic lymphocytic leukemia, acute myelogenous leukemia, chronic myelogenous leukemia, chronic granulocytic leukemia, acute granulocytic leukemia, hairy cell leukemia, neuroblastoma, rhabdomyosarcoma, Kaposi's sarcoma, polycythemia vera, essential thrombocytosis, Hodgkin's disease, non-Hodgkin's lymphoma, soft-tissue sarcoma, osteogenic sarcoma, primary macroglobulinemia, and retinoblastoma. In some embodiments, the cancer is selected from the group of tumor-forming cancers.

In another embodiments the disclosure is provided a method for treating Triple Negative Breast Cancer (TNBC) comprising administering to a subject in need thereof an effective amount a composition comprising an extracellular vesicle as described herein, wherein the composition treats the TNBC. For example, in some embodiments, a first fusion protein comprises a formula A-L₁-B-L₂-C, wherein A is a first antibody moiety, L₁ is a first linker; B is a second antibody, L₂ is a second linker, and C is an exosomal membrane protein transmembrane domain moiety; and a second fusion protein comprises, consists essentially of, or consist of the formula D-E-F, wherein D is a first protein binding moiety, E is a second exosomal membrane protein transmembrane domain, and F is a second protein binding moiety. In other embodiments, the second fusion protein comprises, consists essentially of, or consists of the formula D-L₃-E-L₄-F, wherein D is a first protein binding moiety, L₃ is a third linker, E is an exosomal protein transmembrane domain; L₄ is a fourth linker, and F is a second protein binding moiety, wherein the first and second fusion proteins are displayed on a surface of the engineered extracellular vesicle.

Alternatively, the extracellular vesicle used to treat TNBC or another cancer may include an engineered extracellular vesicle comprising a first fusion protein comprising, consisting essentially of, or consisting of the formula T₁-A-L₁-B-L₂-C-T₂, wherein T₁ is a first epitope tag, A is the first antibody moiety, L₁ is a first linker moiety, B is the second antibody moiety, L₂ is a second linker moiety, C is the first exosomal protein transmembrane domain, and T₂ is a second epitope tag; and the second fusion protein comprises, consists essentially of, or consists of a formula T₃-D-L3-E-L4-F, wherein T3 is a third epitope tag, D is the first protein binding moiety, L₃ is a third linker moiety, E is the second exosomal membrane protein transmembrane domain, L₄ is a fourth linker moiety, and F is the second protein binding moiety. In some embodiments, the first and second fusion proteins are SEQ ID NO: 1 and SEQ ID NO: 2, respectively.

In some embodiments, a fusion protein also may include a signal peptide on the N-terminus of the fusion protein. For example, the first fusion protein may have the formula S₁-T₁-A-L₁-B-L₂-C-T₂, wherein S₁ is a signal peptide, T₁ is a first epitope tag, A is the first antibody moiety, L₁ is a first linker moiety, B is the second antibody moiety, L₂ is a second linker moiety, C is the first exosomal protein transmembrane domain, and T₂ is a second epitope tag.

### Results and Discussion

To determine whether endogenous exosomes could be reprogrammed as novel immunotherapeutics, we sought to genetically engineer exosomes with both targeting moieties and immunomodulatory agents and examine their activities in mounting immune responses using TNBC cellular and animal models in consideration of unmet medical needs for this disease. To this end, single-chain variable fragments (scFvs) targeting human T-cell CD3 and TNBC-associated EGFR were designed as targeting domains for GEMINI-Exos. Two immune checkpoint modulators, PD-1 and OX40L, were selected for display on GEMINI-Exos to augment cellular immunity by competing for PD-1 ligands' binding and activating OX40 signaling pathway, respectively. PD-1 and OX40 are expressed on activated T cells but play opposite roles in regulating T-cell activation. Engagement of PD-1 with its ligands, PD-L1 and PD-L2, produces inhibitory signals, whereas ligation of OX40 by its ligand OX40L generates stimulatory activities.

To balance stability, biological activities, and expression yields of the 4 simultaneously displayed proteins on exosome surfaces, we chose to transfect and co-express two fusion proteins constructs in exosome-producing cells (FIG. 1). By following this strategy, αCD3 scFv and αEGFR scFv could be functionally anchored on exosomal surfaces through tandem fusion with the transmembrane domain (TMD) of human platelet-derived growth factor receptor (PDGFR). A hemagglutinin (HA) epitope tag was placed at N-terminus of αCD3-αEGFR-PDGFR TMD fusion.

PD-1 and OX40L are type I and II membrane proteins, respectively. To display both on exosome surface in a single-chain fusion format with correct orientations, we attempted to genetically link full-length PD-1 and OX40L to N- and C-terminus of CD9, respectively (FIG. 1). Considering the high abundance and intracellular termini for CD9 in exosome membranes, CD9-based fusions may facilitate display of transmembrane proteins on exosome surfaces. The designed PD-1-CD9-OX40L fusion contains an N-terminal HA tag, a C-terminal 6×His tag, and flexible (GGGGS)₂ linkers (SEQ ID NO: 17) before and after the fused CD9 domain.

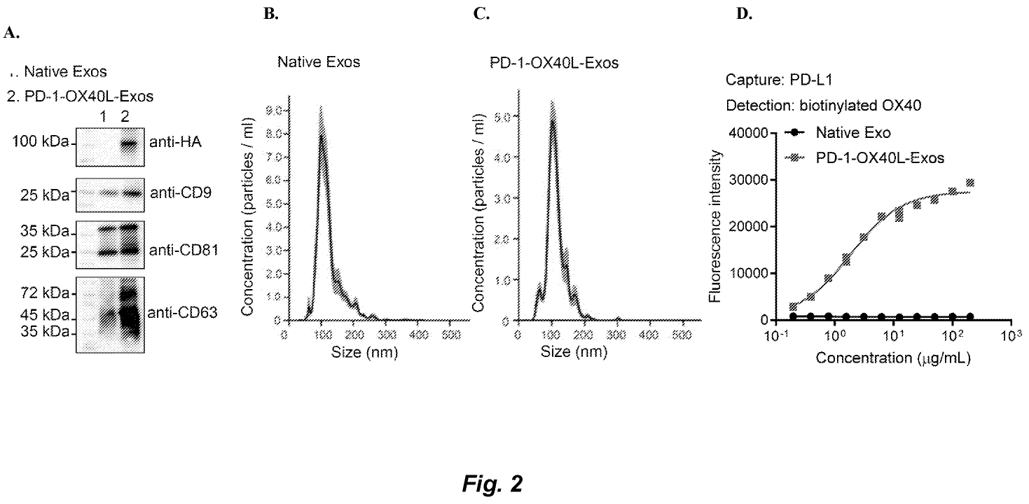

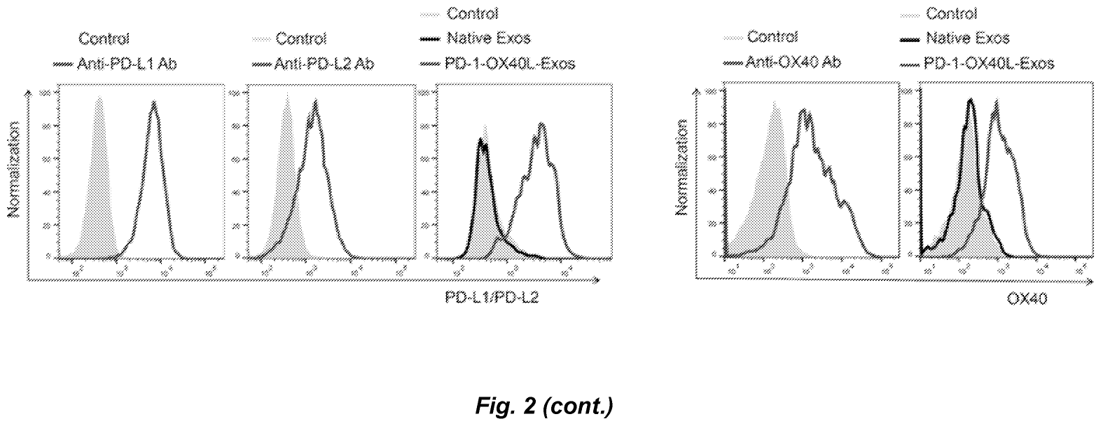

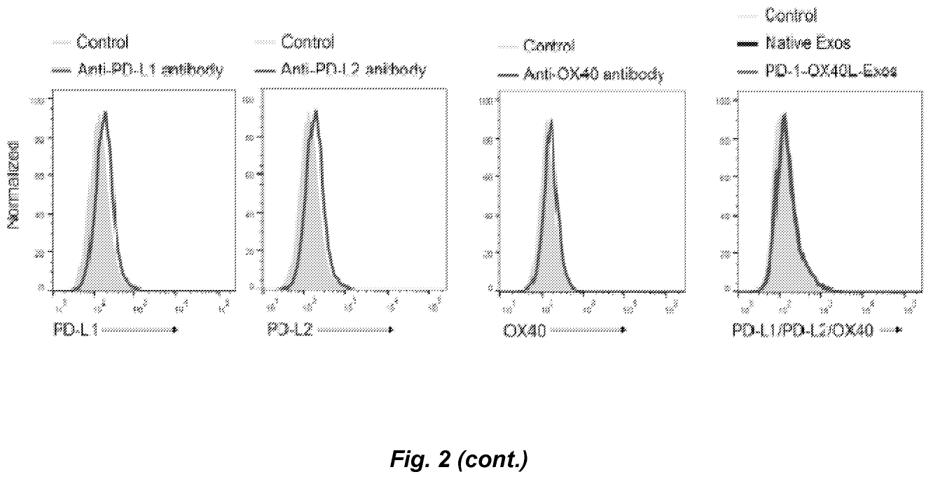

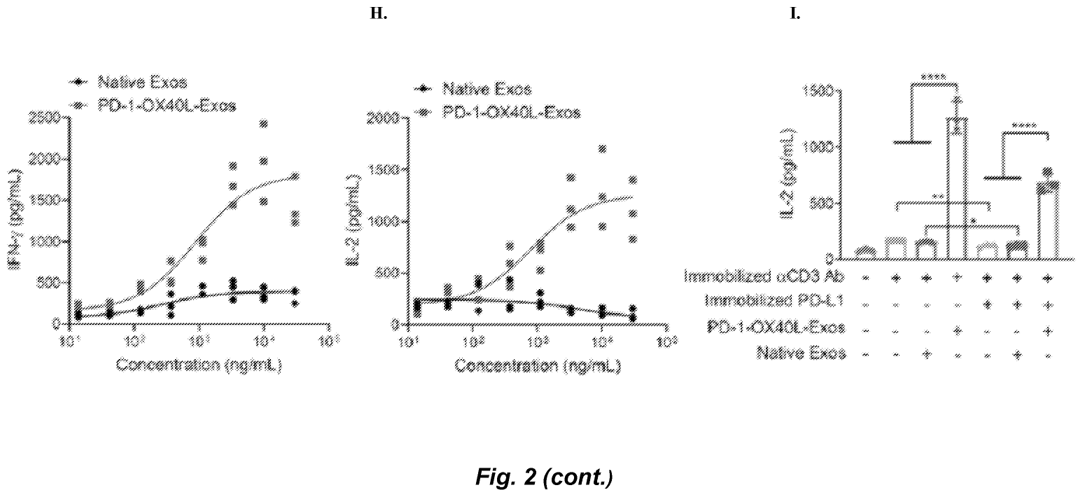

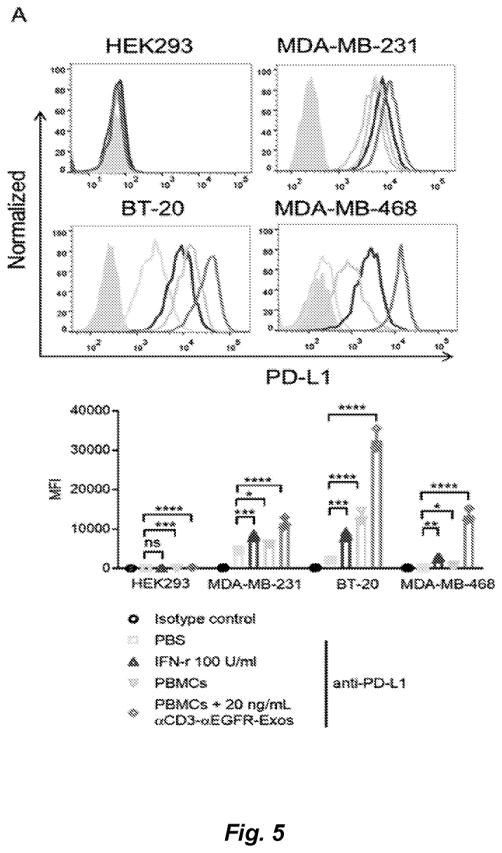

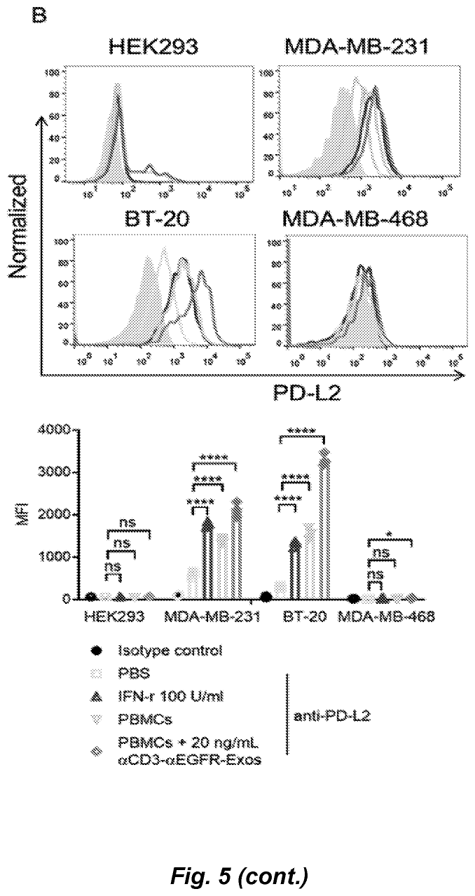

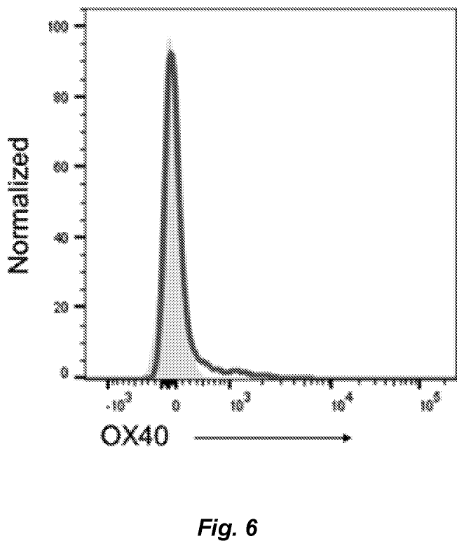

The expression and function of the PD-1-CD9-OX40L fusion protein in exosomes were examined before generating the αCD3-αEGFR-PD-1-OX40L GEMINI-Exos. PD-1-OX40L-Exos were produced through transient transfection of Expi293F cells with the designed fusion construct, followed by purification from harvested media via differential centrifugations and ultracentrifugation. Immunoblot results confirmed expression of the PD-1-CD9-OX40L fusion protein in exosomes (FIG. 2A). Nanoparticle tracking analysis (NTA) revealed that the mean and mode size of PD-1-OX40L-Exos are around 115 nm and 105 nm, respectively, similar to that of native exosomes (FIG. 2B). Sandwich ELISA indicated that unlike native exosomes, the PD-1-OX40L-Exos can simultaneously bind to both the human PD-L1 and OX40 (FIG. 2C). The bindings of PD-1-OX40L-Exos to PD-L1/PD-L2 and OX40 were further analyzed by flow cytometry (FIG. 2D) using BT-20 cells with constitutive and upregulated expression of PD-L1 and PD-L2 upon stimulation (FIGS. 2D and 5), activated human T cells (FIGS. 2E and 6), and negative MDA-MB-468 cells (FIGS. 2F and 5). PD-1-OX40L-Exos display tight binding to both the PD-L1⁺/PD-L2⁺ BT-20 cells and OX40⁺ T cells and no binding to PD-L1⁻/PD-L2⁻/OX40⁻ MDA-MB-468 cells. These results support functional displays of PD-1 and OX40L on exosome surfaces.

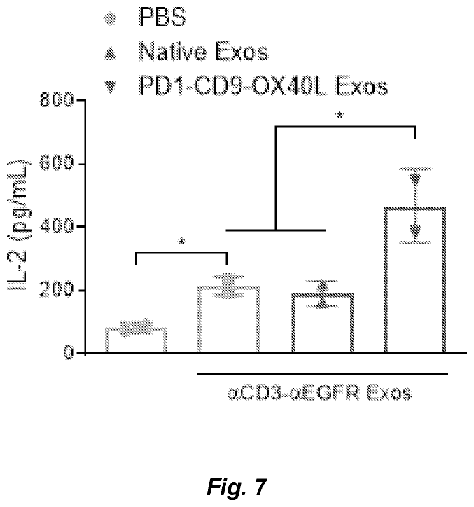

The stimulatory activity of PD-1-OX40L-Exos on activation of primary T cells was then evaluated. Incubation of PD-1-OX40L-Exos with human peripheral blood mononuclear cells (PBMCs) activated primarily by an anti-CD3 antibody result in dose-dependent increases of secreted IFN-γ and IL-2 cytokines (FIG. 2G, H). In contrast, native exosomes have little effects on stimulating T-cell activation. On the basis of secreted IL-2 levels, additions of PD-1-OX40L-Exos restore T-cell activation that is inhibited by immobilized PD-L1 introduced to anti-CD3 antibody-activated PBMCs (FIG. 2I). In addition, αCD3-αEGFR-Exos were prepared as previously described²⁰ and used to recruit and activate human T cells against EGFR⁺ BT-20 TNBC cells in the absence or presence of PD-1-OX40L-Exos. Significantly higher levels of IL-2 release were observed for PBMC:BT-20 mixtures with PD-1-OX40L-Exos (FIG. 7). These results demonstrate in vitro immune stimulating activities for PD-1-OX40L-Exos.

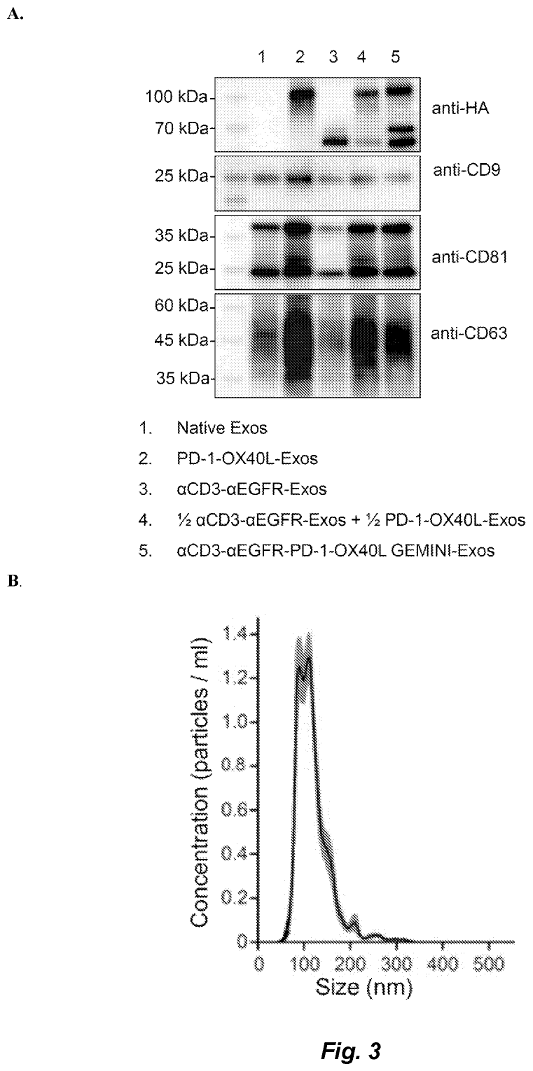

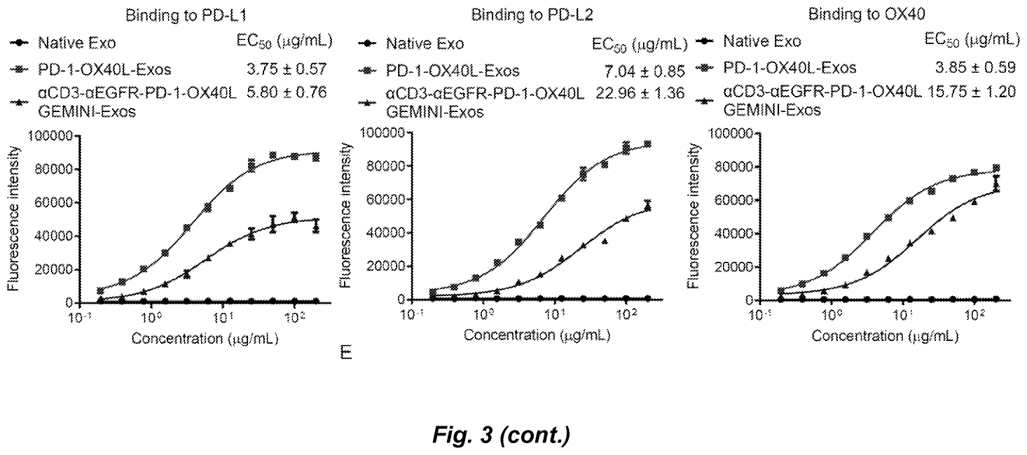

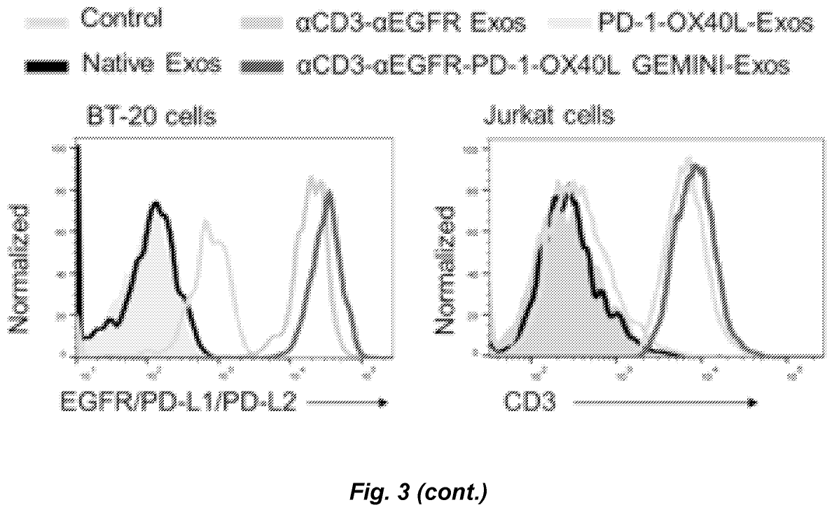

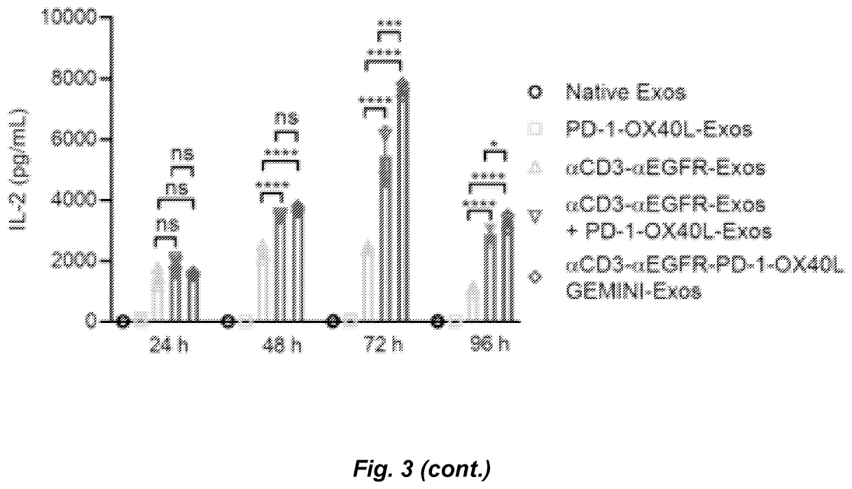

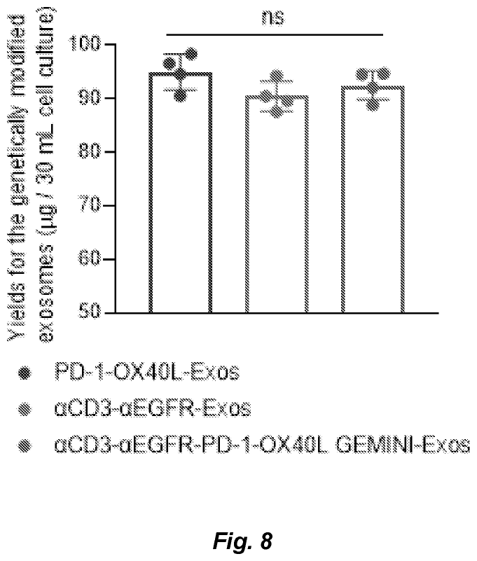

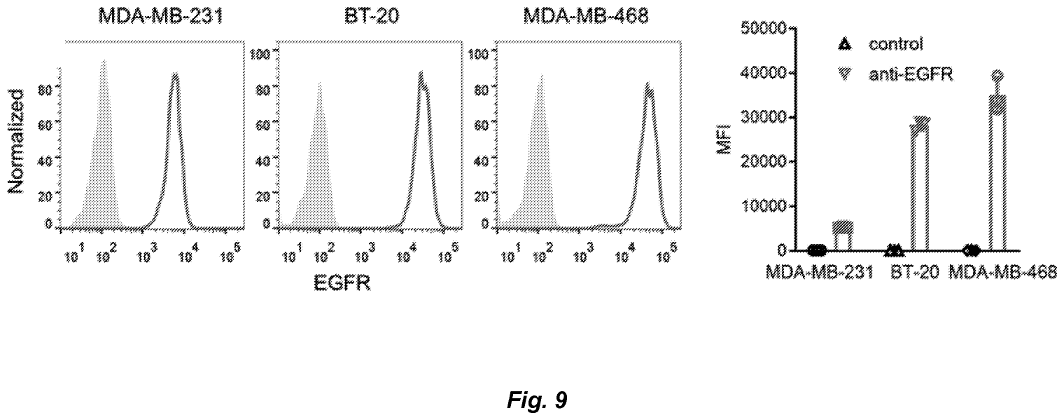

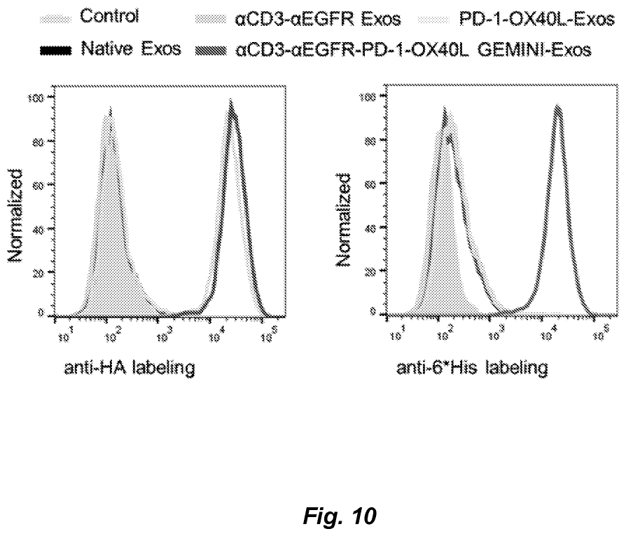

Next, αCD3-αEGFR-PD-1-OX40L GEMINI-Exos were generated by co-transfecting exosome-producing cells with αCD3-αEGFR-PDGFR TMD and PD-1-CD9-OX40L fusion expression constructs. Immunoblot analysis indicated that both fusion proteins were successfully expressed in exosomes (FIG. 3A) and the three genetically modified exosomes showed comparable yields (FIG. 8). The size distribution for GEMINI-Exos is comparable to those of native exosomes and PD-1-OX40L-Exos, according to NTA analysis (FIG. 3B). ELISA results showed that the αCD3-αEGFR-PD-1-OX40L GEMINI-Exos retain strong bindings to PD-L1, PD-L2, and OX40 targets, despite slightly decreased binding affinity in comparison to PD-1-OX40L-Exos (FIG. 3C). Flow cytometry revealed that the GEMINI-Exos exhibit the tightest binding to BT-20 cells (EGFR⁺ PD-L1⁺ PD-L2⁺) compared with αCD3-αEGFR-Exos and PD-1-OX40L-Exos (FIGS. 3D, 5, and 9), possibly due to dual targeting capability to EGFR and PD-L1/L2. The binding affinity of GEMINI-Exos to Jurkat cells (CD3⁺ OX40⁻) is comparable to that of αCD3-αEGFR-Exos (FIG. 3D). Moreover, αCD3-αEGFR-PD-1-OX40L GEMINI-Exos show strong binding to MDA-MB-468 cells (PD-L1⁻ PD-L2⁻ OX40⁻ CD3⁻ EGFR⁺) as detected by an anti-6×His antibody, indicating co-expression of the HA-αCD3-αEGFR-PDGFR TMD and HA-PD-1-CD9-OX40L-6×His fusion proteins on the surface of the same exosome (FIG. 10). These results demonstrate that both targeting antibodies and immunoregulatory proteins are functionally displayed on exosomal surfaces and may allow exosomes to engage TNBC cells by T cells for eliciting cancer-specific cellular immunity. Notably, incubation of human PBMC:BT-20 cell mixtures with the αCD3-αEGFR-PD-1-OX40L GEMINI-Exos results in significantly higher and more sustainable levels of IL-2 release in contrast to ones with αCD3-αEGFR-Exos or a mixture (1:1) of αCD3-αEGFR-Exos and PD-1-OX40L-Exos (FIG. 3E), suggesting the GEMINI-Exos may induce potent anti-cancer immune responses by modulating PD-1 and OX40-associated immune checkpoint pathways.

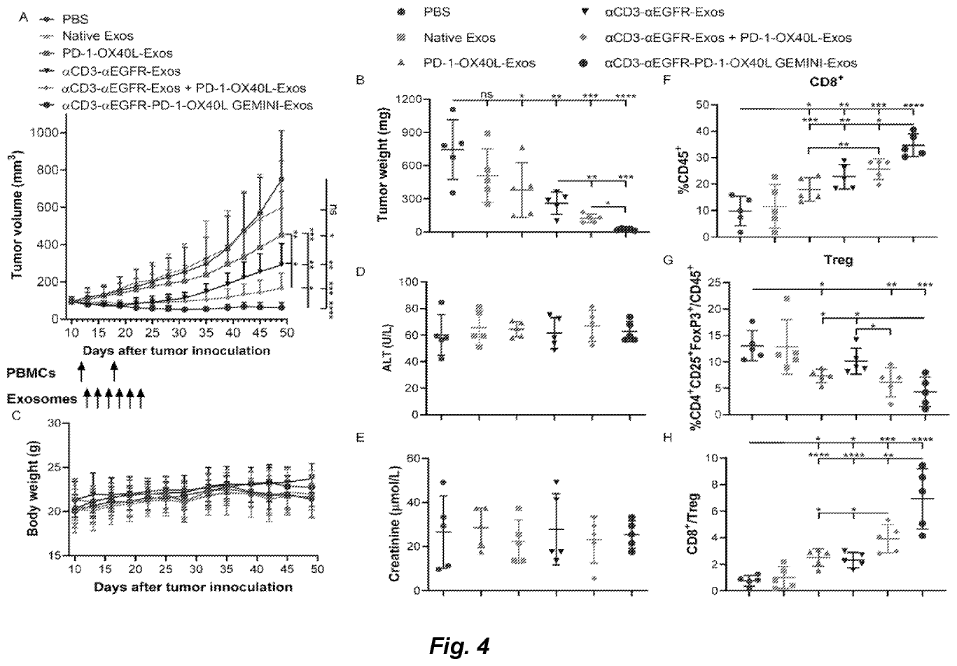

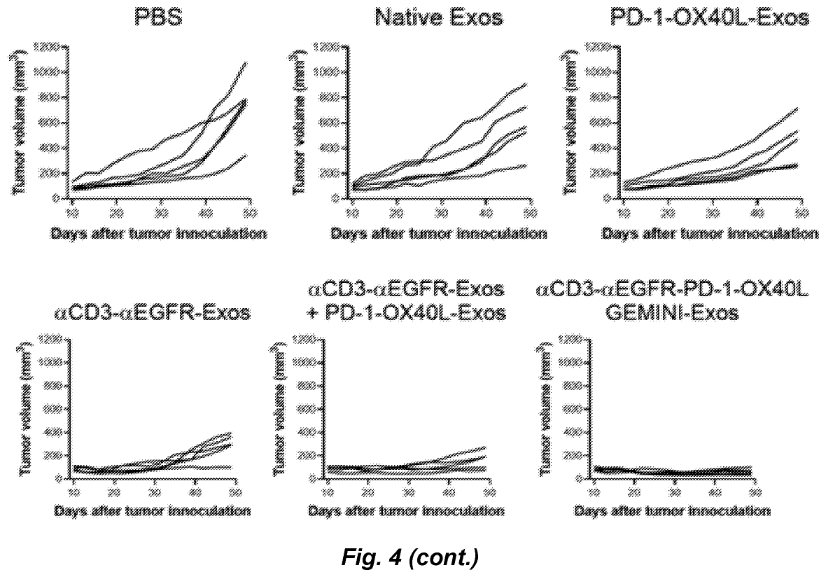

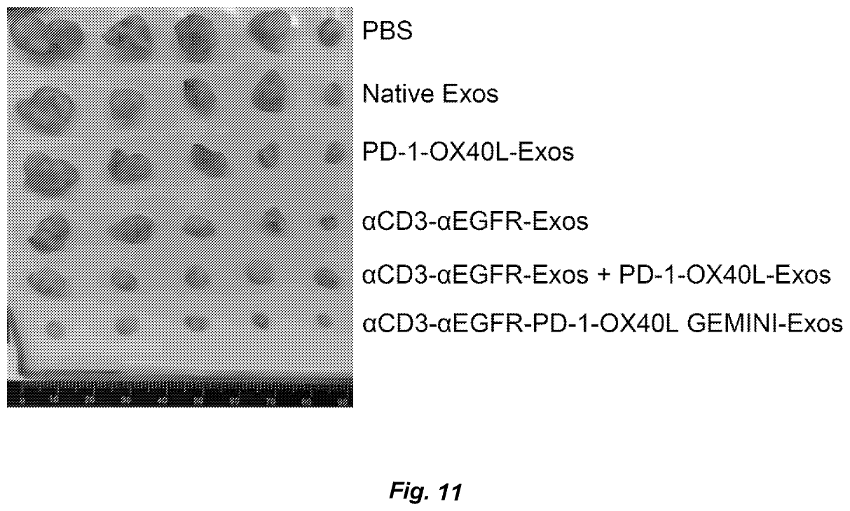

In vivo efficacy of the αCD3-αEGFR-PD-1-OX40L GEMINI-Exos was then evaluated using BT-20 xenograft mouse models with engrafted human PBMCs. As shown in FIGS. 4A and 41, mice treated with PD-1-OX40L-Exos, αCD3-αEGFR-Exos, a mixture (1:1) of PD-1-OX40L-Exos and αCD3-αEGFR-Exos, or GEMINI-Exos display significant growth inhibition against established tumors in comparison to mice treated with PBS or native exosomes. Combination of PD-1-OX40L-Exos and αCD3-αEGFR-Exos shows enhanced anti-tumor efficacy compared with PD-1-OX40L-Exos. Importantly, mice treated with αCD3-αEGFR-PD-1-OX40L GEMINI-Exos exhibit the most pronounced tumor growth inhibition. The weights and photographs of collected tumors at the endpoint are consistent with these results (FIGS. 4B and 11). No overt toxicity or loss in body weight was observed for mice in all groups (FIG. 4C). In addition, no significant changes were found for alanine aminotransferase (ALT) activities (a liver injury marker) and creatinine concentrations (a kidney injury marker) in plasma across all groups at the end of studies (FIG. 4D, E). These results indicate the excellent anti-tumor activity and safety for αCD3-αEGFR-PD-1-OX40L GEMINI-Exos.

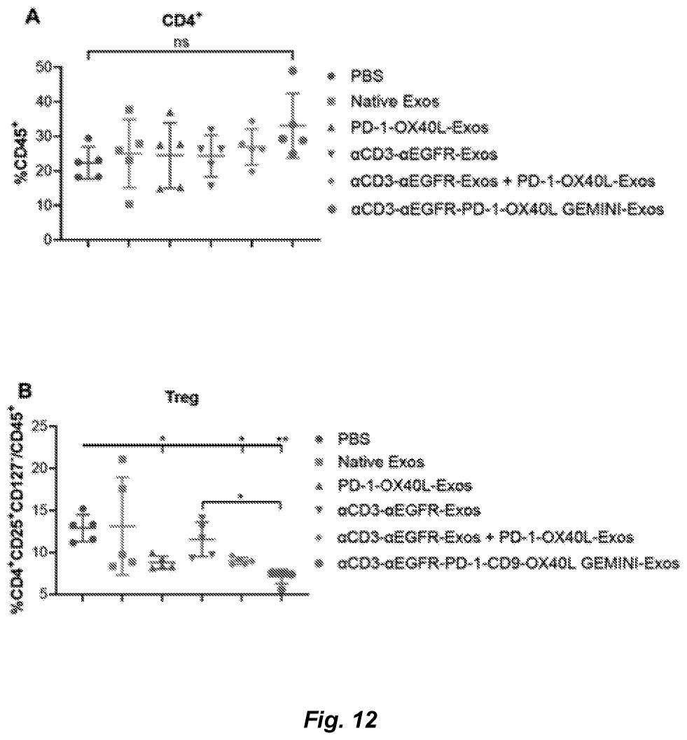

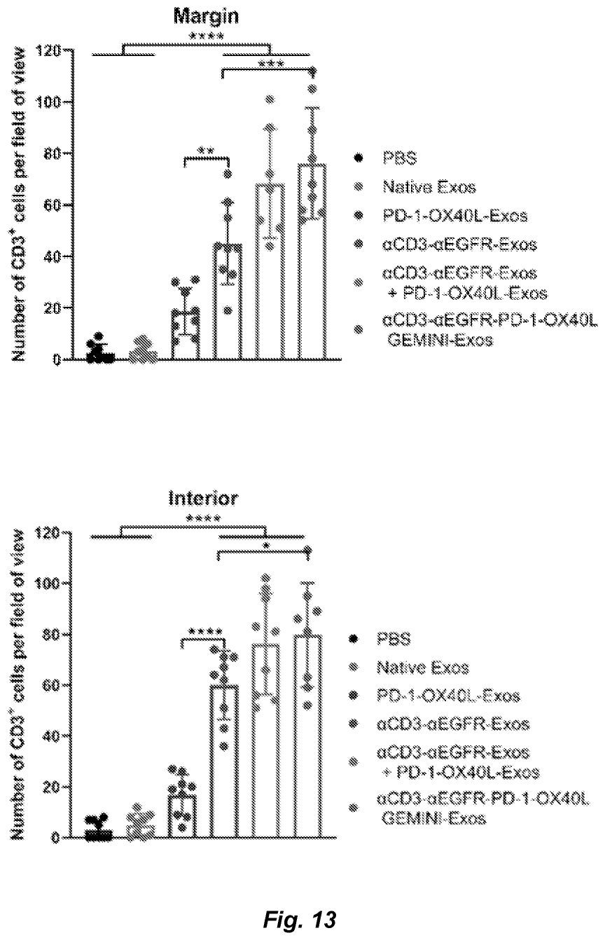

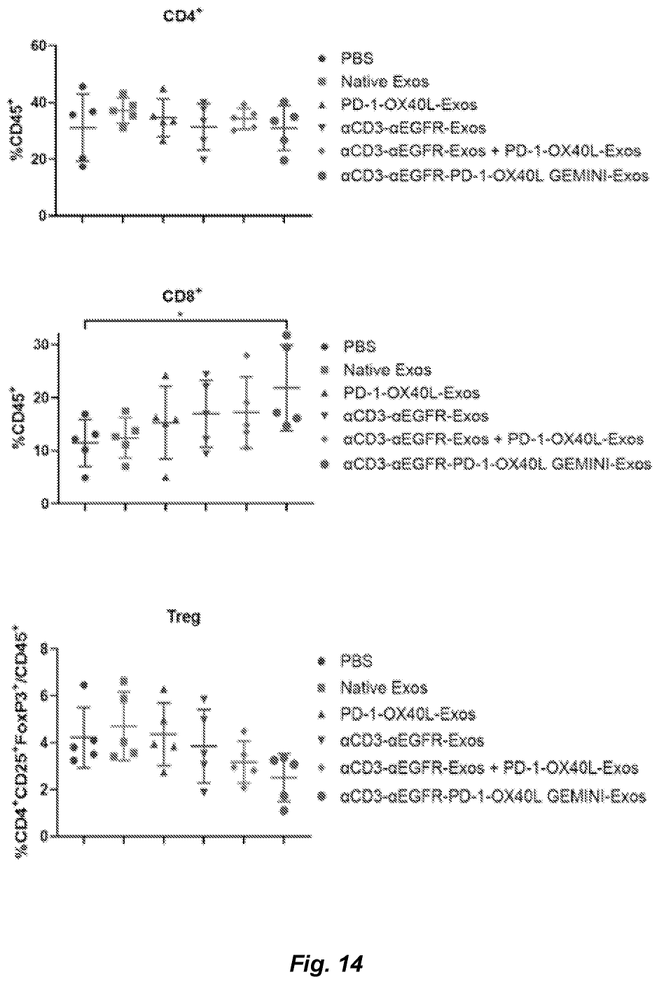

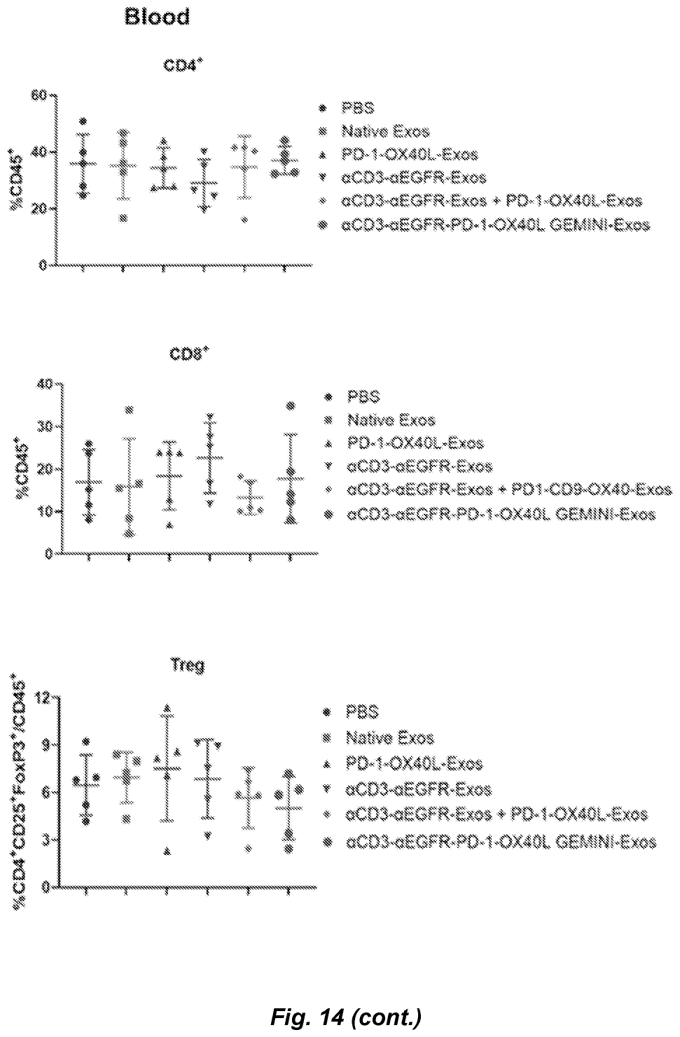

Tumor infiltrating lymphocytes were harvested and analyzed at the endpoint (FIGS. 4F-H and 12-15). Compared with PBS- or native exosomes-treated groups, mice administered with PD-1-OX40L-Exos, αCD3-αEGFR-Exos, the mixture (1:1) of PD-1-OX40L-Exos and αCD3-αEGFR-Exos, or GEMINI-Exos show significantly increased intratumoral CD8⁺ T cells (FIG. 4F). Tregs reduction in tumors were also seen for mice treated with exosomes expressing the PD-1-CD9-OX40L fusion (FIGS. 4G and 14B), supporting immunostimulatory roles for exosomal surface-displayed PD-1 and OX40L in tumor microenvironment. Consistent with anti-tumor efficacy results, GEMINI-Exos-treated mice displayed the most significant changes in tumor-infiltrating CD8⁺ T cells and CD8⁺ T cell/Tregs ratios among all the treatment groups (FIGS. 4F-H and 14B). Mice treated with GEMINI-Exos showed slight but not significant increases of CD4⁺ T cells in tumors (FIG. 12A). Moreover, immunohistofluorescence imaging showed marked infiltration of T cells for tumors implanted in mice receiving αCD3-αEGFR-Exos, the mixture (1:1) of PD-1-OX40L-Exos and αCD3-αEGFR-Exos, or GEMINI-Exos (FIG. 13). In addition, moderate increases of CD8⁺ T cells were founded in the spleen of mice receiving GEMINI-Exos and no significant differences for T-cell subsets in blood were seen across all groups (FIG. 14). These results suggest that the αCD3-αEGFR-PD-1-OX40L GEMINI-Exos induce potent anti-tumor immune responses through promoting CD8⁺ T-cell infiltration and depleting immunosuppression of Tregs.

Cell-derived exosomes have been widely utilized for the delivery of various types of cargos including chemotherapeutics, interfering RNAs, peptides, and proteins. Meanwhile, genetically modified exosomes have been emerging as a new and increasingly important class of therapeutic modality, such as SIRPα-exosomes to block CD47 and increase cancer cell phagocytosis, exoIL-12 to stimulate local and systemic anti-tumor activity, and Exo-PH20 to penetrate deeply into tumor foci via hyaluronan degradation. In this study, native exosomes were genetically modified to express four distinct proteins on surfaces. By simultaneously targeting tumor-associated EGFR and immunomodulatory molecules, the rationally designed αCD3-αEGFR-PD-1-OX40L GEMINI-Exos exhibit excellent activity in directing, activating, and modulating T cell-mediated immunity against EGFR-positive TNBC tumors. To the best of our knowledge, this work represents the first report of successful generation of multifunctional exosomes via genetic engineering approaches for targeted cancer immunotherapy.

In comparison to molecular immunotherapeutics such as bispecific antibodies and immune checkpoint inhibitors, GEMINI-Exos with integrated immunoregulatory proteins are likely to augment therapeutic efficacy by engaging and modulating multiple immune checkpoint pathways. Despite improved efficacy for combination therapies that can target different immunomodulators, physically restricting these therapeutic agents on the same vesicle may facilitate their synergistic actions on individual target cells, resulting increased potency. The GEMINI-Exos feature full-length transmembrane proteins displayed via CD9-fusion. Unlike physical and chemical methods, this genetic approach for incorporating functional proteins into exosome membranes may enable to retain their native folding, generating engineered exosomes with desired functions and properties. And these intact membrane proteins on GEMINI-Exos may possess higher stability and activities than those on synthetic nanoparticles. Furthermore, GEMINI-Exos' functions can be further expanded through packing with small-molecule and nucleic acid agents to improve therapeutic efficacy by leveraging their potential for intracellular drug delivery. In addition, GEMINI-Exos-based therapeutics may show higher biocompatibility than synthetic and viral nanomedicines.

To more effectively fight tumors, immunotherapeutic candidates are required to not only recruit immune effector cells but also sustain anti-tumor immunity in response to dynamic immunosuppressive tumor microenvironments. The GEMINI-Exos were designed to meet these requirements. Surfaced-displayed αCD3 and αEGFR antibodies can redirect cytotoxic T cells toward attacking EGFR-positive TNBC tumors. PD-1 expressed on GEMINI-Exos is expected to block immune checkpoint inhibitory pathway activated by upregulated PD-L1/L2 on tumor surfaces. Multivalent OX40L on GEMINI-Exos is anticipated to engage with OX40 expressed on activated T cells to turn on immune checkpoint stimulatory signals. Furthermore, co-expressed PD-1-OX40L fusion and αCD3-αEGFR antibodies on the surface of the same GEMINI-Exos may increase targeting capabilities toward EGFR-, PD-L1-, PD-L2-positive tumor cells and CD3- and OX40-positive T cells as well as maximize cellular immunity against tumors. Collectively, these GEMINI-Exos-enabled molecular interactions establish robust and sustainable immune responses specific for TNBC tumors.

Exosomes are defined as extracellular vesicles (EVs) that originate as intraluminal vesicles within multivesicular bodies (MVBs) and are secreted upon fusion of the MVBs with plasma membranes. As the most commonly used method for exosome isolation currently, differential ultracentrifugation was employed in this study. The purified GEMINI-Exos are likely to carry other types of EVs with similar morphology, size, and protein expression.

Depending on the cells of origin, exosomes may possess immunomodulatory potentials. Expi293F cells, a suspension-adapted HEK293 cell line, were used here to produce GEMINI-Exos. Exosomes from HEK293 cells were shown to have minimal toxicity and immunogenicity, representing an excellent source of exosomes for the additions of new functions. Moreover, the use of Expi293F cells may facilitate the bioreactor-based, large-scale production of clinical grade exosomes.

In comparison to PDGFR TMD-based fusions, CD9 tetraspanin allows to display both type I and II transmembrane proteins on exosome surface in native orientations, expanding choices of functional proteins for exosome expression. But the extracellular loops of CD9 could impact binding affinity and specificity of the fused protein. Further engineering and optimization may be required for improving biological activities of proteins displayed by CD9. Moreover, the generated GEMINI-Exos can be loaded with therapeutic agents targeting other immune checkpoint pathways for augmented efficacy. Further in vivo studies with different animal models are also needed to evaluate biodistribution, therapeutic efficacy, systemic toxicity, and animal survival for PD-1-OX40L-Exos, αCD3-αEGFR-Exos, the combination of PD-1-OX40L-Exos and αCD3-αEGFR-Exos, and GEMINI-Exos. Improvement of pharmacokinetics and exploration of different administration methods could also be carried out to optimize pharmacological activities of GEMINI-Exos for clinical translation. Notably, development of stable cell lines expressing optimized fusion constructs can further facilitate studies of biological and pharmacological activities of GEMINI-Exos and streamline production of pharmaceutical-grade exosomes. In addition, GEMINI-Exos-based immunotherapeutics for other human cancers and diseases can be developed by extending to different disease-associated antigens and immunoregulatory molecules.

In summary, we designed and generated novel αCD3-αEGFR-PD-1-OX40L GEMINI-Exos by genetically displaying both the monoclonal antibodies and immunomodulatory proteins on exosome surfaces. The generated GEMINI-Exos can not only recruit and activate human T cells against EGFR-positive tumors but also induce robust cancer-specific immune responses, leading to remarkable in vivo anti-tumor efficacy. This work demonstrates the potential for GEMINI-Exos in cancer immunotherapy and may provide a general approach for the development of new immunotherapeutic exosomes with desired pharmacological activities.

### Pharmaceutical Formulations

The compounds described herein can be used to prepare therapeutic pharmaceutical compositions, for example, by combining the compounds with a pharmaceutically acceptable diluent, excipient, or carrier. The compounds may be added to a carrier in the form of a salt or solvate. For example, in cases where compounds are sufficiently basic or acidic to form stable nontoxic acid or base salts, administration of the compounds as salts may be appropriate. Examples of pharmaceutically acceptable salts are organic acid addition salts formed with acids that form a physiologically acceptable anion, for example, tosylate, methanesulfonate, acetate, citrate, malonate, tartrate, succinate, benzoate, ascorbate, a-ketoglutarate, and P-glycerophosphate. Suitable inorganic salts may also be formed, including hydrochloride, halide, sulfate, nitrate, bicarbonate, and carbonate salts.

Pharmaceutically acceptable salts may be obtained using standard procedures well known in the art, for example by reacting a sufficiently basic compound such as an amine with a suitable acid to provide a physiologically acceptable ionic compound. Alkali metal (for example, sodium, potassium or lithium) or alkaline earth metal (for example, calcium) salts of carboxylic acids can also be prepared by analogous methods.

The compounds of the formulas described herein can be formulated as pharmaceutical compositions and administered to a mammalian host, such as a human patient, in a variety of forms. The forms can be specifically adapted to a chosen route of administration, e.g., oral or parenteral administration, by intravenous, intramuscular, topical or subcutaneous routes.

The compounds described herein may be systemically administered in combination with a pharmaceutically acceptable vehicle, such as an inert diluent or an assimilable edible carrier. For oral administration, compounds can be enclosed in hard or soft-shell gelatin capsules, compressed into tablets, or incorporated directly into the food of a patient's diet. Compounds may also be combined with one or more excipients and used in the form of ingestible tablets, buccal tablets, troches, capsules, elixirs, suspensions, syrups, wafers, and the like. Such compositions and preparations typically contain at least 0.1% of active compound. The percentage of the compositions and preparations can vary and may conveniently be from about 0.5% to about 60%, about 1% to about 25%, or about 2% to about 10%, of the weight of a given unit dosage form. The amount of active compound in such therapeutically useful compositions can be such that an effective dosage level can be obtained.

The tablets, troches, pills, capsules, and the like may also contain one or more of the following: binders such as gum tragacanth, acacia, corn starch or gelatin; excipients such as dicalcium phosphate; a disintegrating agent such as corn starch, potato starch, alginic acid and the like; and a lubricant such as magnesium stearate. A sweetening agent such as sucrose, fructose, lactose or aspartame; or a flavoring agent such as peppermint, oil of wintergreen, or cherry flavoring, may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propyl parabens as preservatives, a dye and flavoring such as cherry or orange flavor. Any material used in preparing any unit dosage form should be pharmaceutically acceptable and substantially non-toxic in the amounts employed. In addition, the active compound may be incorporated into sustained-release preparations and devices.

The active compound may be administered intravenously or intraperitoneally by infusion or injection. Solutions of the active compound or its salts can be prepared in water, optionally mixed with a nontoxic surfactant. Dispersions can be prepared in glycerol, liquid polyethylene glycols, triacetin, or mixtures thereof, or in a pharmaceutically acceptable oil. Under ordinary conditions of storage and use, preparations may contain a preservative to prevent the growth of microorganisms.

Pharmaceutical dosage forms suitable for injection or infusion can include sterile aqueous solutions, dispersions, or sterile powders comprising the active ingredient adapted for the extemporaneous preparation of sterile injectable or infusible solutions or dispersions, optionally encapsulated in liposomes. The ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions, or by the use of surfactants. The prevention of the action of microorganisms can be brought about by various antibacterial and/or antifungal agents, for example, parabens, chlorobutanol, phenol, sorbic acid, thimerosal, and the like. In many cases, it will be preferable to include isotonic agents, for example, sugars, buffers, or sodium chloride. Prolonged absorption of the injectable compositions can be brought about by agents delaying absorption, for example, aluminum monostearate and/or gelatin.

Sterile injectable solutions can be prepared by incorporating the active compound in the required amount in the appropriate solvent with various other ingredients enumerated above, as required, optionally followed by filter sterilization. In the case of sterile powders for the preparation of sterile injectable solutions, methods of preparation can include vacuum drying and freeze-drying techniques, which yield a powder of the active ingredient plus any additional desired ingredient present in the solution.

For topical administration, compounds may be applied in pure form, e.g., when they are liquids. However, it will generally be desirable to administer the active agent to the skin as a composition or formulation, for example, in combination with a dermatologically acceptable carrier, which may be a solid, a liquid, a gel, or the like.

Useful solid carriers include finely divided solids such as talc, clay, microcrystalline cellulose, silica, alumina, and the like. Useful liquid carriers include water, dimethyl sulfoxide (DMSO), alcohols, glycols, or water-alcohol/glycol blends, in which a compound can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using a pump-type or aerosol sprayer.

Thickeners such as synthetic polymers, fatty acids, fatty acid salts and esters, fatty alcohols, modified celluloses, or modified mineral materials can also be employed with liquid carriers to form spreadable pastes, gels, ointments, soaps, and the like, for application directly to the skin of the user.

Examples of dermatological compositions for delivering active agents to the skin are known to the art; for example, see U.S. Pat. Nos. 4,992,478, 4,820,508, 4,608,392, and 4,559,157. Such dermatological compositions can be used in combinations with the compounds described herein where an ingredient of such compositions can optionally be replaced by a compound described herein, or a compound described herein can be added to the composition.

Useful dosages of the compounds described herein can be determined by comparing their in vitro activity, and in vivo activity in animal models. Methods for the extrapolation of effective dosages in mice, and other animals, to humans are known to the art; for example, see U.S. Pat. No. 4,938,949 (Borch et al.). The amount of a compound, or an active salt or derivative thereof, required for use in treatment will vary not only with the particular compound or salt selected but also with the route of administration, the nature of the condition being treated, and the age and condition of the patient, and will be ultimately at the discretion of an attendant physician or clinician.

In general, however, a suitable dose will be in the range of from about 0.5 to about 100 mg/kg, e.g., from about 10 to about 75 mg/kg of body weight per day, such as 3 to about 50 mg per kilogram body weight of the recipient per day, preferably in the range of 6 to 90 mg/kg/day, most preferably in the range of 15 to 60 mg/kg/day.

The compound is conveniently formulated in unit dosage form; for example, containing 5 to 1000 mg, conveniently 10 to 750 mg, most conveniently, 50 to 500 mg of active ingredient per unit dosage form. In one embodiment, the invention provides a composition comprising a compound of the invention formulated in such a unit dosage form.

The compound can be conveniently administered in a unit dosage form, for example, containing 5 to 1000 mg/m², conveniently 10 to 750 mg/m², most conveniently, 50 to 500 mg/m² of active ingredient per unit dosage form. The desired dose may conveniently be presented in a single dose or as divided doses administered at appropriate intervals, for example, as two, three, four or more sub-doses per day. The sub-dose itself may be further divided, e.g., into a number of discrete loosely spaced administrations.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations; such as multiple inhalations from an insufflator or by application of a plurality of drops into the eye.

The invention provides therapeutic methods of treating cancer in a vertebrate such as a mammal, which involve administering to a mammal having cancer an effective amount of a compound or composition described herein. A mammal includes a primate, human, rodent, canine, feline, bovine, ovine, equine, swine, caprine, bovine and the like. Cancer refers to any of the various type of malignant neoplasm, which are in general characterized by an undesirable cellular proliferation, e.g., unregulated growth, lack of differentiation, local tissue invasion, and metastasis. Cancers that can be treated by a compound described herein include, for example, breast cancer, cervical carcinoma, colon cancer, endometrial cancer, leukemia, lung cancer, melanoma, pancreatic cancer, prostate cancer, ovarian cancer, or uterine cancer, and in particular, any cancer that is ERα positive.

The ability of a compound of the invention to treat cancer may be determined by using assays well known to the art. For example, the design of treatment protocols, toxicity evaluation, data analysis, quantification of tumor cell kills, and the biological significance of the use of transplantable tumor screens are known. In addition, ability of a compound to treat cancer may be determined using the Tests as described below.

The following Examples are intended to illustrate the above invention and should not be construed as to narrow its scope. One skilled in the art will readily recognize that the Examples suggest many other ways in which the invention could be practiced. It should be understood that numerous variations and modifications may be made while remaining within the scope of the invention.

### EXAMPLES

### Example 1. Materials and Methods

Materials. Dulbecco's modified Eagle's medium (DMEM), Roswell Park Memorial Institute (RPMI) 1640 medium, and Dulbecco's phosphate buffered saline (DPBS) were purchased from Corning Inc. (Corning, NY). BalanCD HEK293 medium and L-glutamine (200 mM) were purchased from FUJIFILM Irvine Scientific. Fetal bovine serum (FBS), Opti-modified Eagle's medium (Opti-MEM), QuantaBlu fluorogenic peroxidase substrate, and Coomassie Plus (Bradford) assay kit were purchased from Thermo Fisher Scientific (Waltham, MA). Collagenase (Type II) was purchased from Worthington Biochemical Corporation. DNase I, Triton X-100, sodium pyruvate, alanine, sodium 2-oxoglutarate monobasic, 2, 4-dinitrophenylhydrazine (2,4-DNPH), picric acid solution (1.3% in H₂O), trichloroacetic acid (TCA), and creatinine were purchased from Sigma-Aldrich (St. Louis, MO).

Cell lines. Human peripheral blood mononuclear cells (PBMCs) were purchased from HemaCare (Van Nuys, CA). Human embryonic kidney 293 cells (HEK293) were purchased from the American Type Culture Collection (ATCC) (Manassas, VA) and maintained in DMEM medium supplemented with 10% FBS at 37° C. in 5% C02. Breast cancer cell lines MDA-MB-468 and MDA-MB-231 were obtained from ATCC and maintained in RPMI 1640 medium supplemented with 10% FBS at 37° C. in 5% C02. BT-20 cell line was purchased from ATCC and cultured in MEM with 10% FBS at 37° C. in 5% C02. Expi293F cells were purchased from Thermo Fisher Scientific (Waltham, MA) and maintained in BalanCD HEK293 medium with 4 mM L-glutamine with shaking at a speed of 125 rpm min⁻¹ at 37° C. in 8% C02.

Molecular cloning and expression of engineered exosomes in mammalian cells. All DNA fragments and PCR primers used in this study are listed in Table 1.

TABLE 1
List of primer
sequences used for molecular cloning.

| DNA fragment | |
|---|---|
| for cloning | Primer sequence |
| PD-1 | Forward: |
| | 5′-CAGTGTGCTGGAATTCGGCTTGGGGAT |
| | ATCCACC-3′ (SEQ ID NO: 41) |
| | Reverse: |
| | 5′-CCGGACTACCACCGCCTCCGCTAGCGA |
| | GGGGCCAAGAGCAGTGTCCATCC-3′ |
| | (SEQ ID NO: 42) |
| CD9-OX40L | Forward: |
| | 5′-CCCTCGCTAGCGGAGGCGGTGGTAGTC |
| | CGGTCAAAGGAGGCACCAAGTGCATCAAAT |
| | ACC-3′ (SEQ ID NO: 43) |
| | Reverse: |
| | 5′-GATCTCGAGCGGCCGCCTTAATGGTGG |
| | TGGTGATGGTGAAGG-3′ |
| | (SEQ ID NO: 44) |
| PD-1-CD9- | Forward: |
| OX40L | 5′-CAGTGTGCTGGAATTCGGCTTGGGGAT |
| | ATCCACC-3′ (SEQ ID NO: 45) |
| | Reverse: |
| | 5′-GATCTCGAGCGGCCGCCTTAATGGTGG |
| | TGGTGATGGTGAAGG-3′ |
| | (SEQ ID NO: 46) |

Restriction enzyme sites EcoRI and NotI are underlined and italicized.

PD-1 fragment was amplified from human PD-1 cDNA purchased from Horizon Discovery Ltd. (clone ID: 6147966) with a hemagglutinin (HA)-tag fused at N-terminus. Synthetic genes encoding CD9-OX40L-6×His was purchased from Integrated DNA Technologies, Inc. (Skokie, IL) with a (GGGGS)₂ linker and a SalI restriction enzyme site inserted between CD9 and OX40L. To generate HA-PD-1-CD9-OX40L-6×His fusion gene fragment, overlap extension polymerase chain reactions (PCR) was performed and a GGGGS linker (SEQ ID NO: 36) and a NheI restriction enzyme site were inserted between PD-1 and CD9 fragments. The amplified HA-PD-1-CD9-OX40L-6×His fragment was ligated in-frame using T4 DNA ligase (New England Biolabs, Ipswich, MA) between the EcoRI and NotI restriction enzyme sites in a modified pDisplay vector in which the N-terminal signal peptide and the transmembrane domain (TMD) of human platelet-derived growth factor receptor (PDGFR) were deleted. The generated expression vector pDisplay-PD-1-CD9-OX40L was confirmed by DNA sequencing provided by GENEWIZ (South Plainfield, NJ). The construct pDisplay-αCD3-αEGFR-PDGFR TMD was generated as described in Cheng et al., J Am Chem Soc 2018, 140 (48), 16413-16417 and Cheng et al., Methods Mol. Biol. 2020, 2097, 197-209. Transfection-level plasmids for the sequence-verified expression constructs were purified using ZymoPURE II plasmid kits (ZYMO Research, Irvine, CA) and transiently transfected into Expi293F cells using PEI MAX 40K (Polysciences, PA) by following manufacturer's instructions. Cell culture supernatants were collected on day 3 and day 6 post transfection through centrifugation and stored at −80° C.

Exosomes purification. Exosomes were isolated from cell culture supernatants of Expi293F cells through differential centrifugation and ultracentrifugation as previously described with modifications. Briefly, cell cultures were first centrifuged at 100×g for 10 minutes to remove Expi293F cells and then centrifuged at 4000×g for 30 minutes to remove dead cells and cell debris using a Heraeus Megafuge 40R refrigerated centrifuge with a TX-750 swinging bucket rotor (Thermo Fisher Scientific). The collected supernatant was then centrifuged at 14,000×g for 40 minutes by J2-21 floor model centrifuge with a JA-17 fixed-angle aluminum rotor (Beckman Coulter, Indianapolis, IN) to remove large vesicles. Clarified supernatants were then centrifuged in a Type 70 Ti rotor by Optima L-80 XP ultracentrifuge (Beckman Instruments) at 60,000 rpm (371,000 xg) for 1.5 hours to pellet exosomes. All the centrifuge processes were performed at 4° C. The resulting exosome pellets were washed twice with PBS, resuspended in PBS, and followed by filtration using 0.2 μm syringe filters. Protein concentrations of the purified exosomes were determined by Bradford assays by following manufacturer's instructions.

Nanoparticle tracking analysis (NTA). The size distribution and concentration of the purified exosomes were determined through NTA using a Nanosight LM10 (Malvern Instruments, U.K.) by following the manufacturer's instructions. Ten replicates of analysis with 60 seconds for each were performed.

Immunoblot analysis. Western blots were performed as previously described.^{1,2} Briefly, purified exosome were lysed in NuPAGE LDS sample buffer (Thermo Fisher Scientific, MA) with or without 10 mM dithiothreitol and boiled at 95° C. for 5 min. The lysates were then resolved by 4-20% ExpressPlus-PAGE gels (GeneScript, Piscataway, NJ), transferred to Immun-Blot PVDF membranes (Bio-Rad Laboratories, Inc, Hercules, CA), blocked with 5% bovine serum albumin (BSA) (Thermo Fisher Scientific, MA) in PBS with 0.5% Tween-20 (PBST) and probed with appropriate primary antibodies (anti-HA (2-2.2.14) from Thermo Fisher Scientific, anti-CD9 (D8O1A) from Cell Signaling Technology, anti-CD81 (5A6) and anti-CD63 (H5C6) from BioLegend) and secondary antibodies (anti-mouse IgG-HRP (62-6520) and anti-rabbit IgG-HRP (65-6120) from Thermo Fisher Scientific). HA-tagged fusion proteins and CD9 were resolved under fully denaturing and reducing conditions, while CD81 and CD63 were resolved under non-reducing conditions. The immunoblots were developed by additions of SuperSignal West Pico PLUS chemiluminescent substrate (Thermo Fisher Scientific) and imaged with a ChemiDoc Touch Imaging System (Bio-Rad Laboratories, Inc, Hercules, CA).

ELISA analysis of binding of engineered exosomes to ligands and receptors. The bindings of PD-1-OX40L-Exos and αCD3-αEGFR-PD-1-OX40L GEMINI-Exos to PD-L1, PD-L2, and OX40 were determined by immunocapture-based ELISA. High-binding 96 well plates (Greiner Bio-One, Monroe, NC) were coated with various concentrations of exosomes overnight at room temperature. Following extensive washing with PBST, wells were blocked with PBS containing 1% BSA for 2 hours at room temperature, followed by extensive washing with PBST. Corresponding ligands or receptors (0.4 g mL⁻¹; PD-L1-hFc and PD-L2-hFc from PeproTech, Inc. and OX40-hFc from BioLegend) were added and incubated for 2 hours at room temperature, followed by extensive washing with PBST. Goat anti-human IgG-HRP was subsequently added for 1-hour incubation at room temperature, followed by extensive washing. QuantaBlu fluorogenic peroxidase substrate (Thermo Fisher Scientific, MA) was then added. Fluorescence intensities (Ex: 325 nm; Em: 420 nm) were measured using a BioTek Synergy H1 Hybrid Multi-Mode Microplate reader (BioTek, VT).

To detect the binding of PD-1-OX40L-Exos to both PD-L1 and OX40. ELISA was performed as described above with PD-L1-hFc (0.4 g mL⁻¹) as the capture protein and biotinylated OX40-hFc as the detection reagent.

Flow cytometric analysis of binding of engineered exosomes to target-expressing cells. BT-20 cells were treated with 100 U/mL IFN-γ (BioLegend) for 2 days to induce expression of PD-L1. Purified human PBMCs from HemaCare were treated with immobilized anti-CD3 antibody (5 μg mL⁻¹; clone: OKT3 from BioLegend) and soluble anti-CD28 antibody (2 μg mL⁻¹; clone: CD28.2, from BioLegend) for 2 days to induce expression of OX40. PD-L1-expressing BT-20 cells, OX40-expressing PBMCs, and Jurkat cells were then used to verify the binding of PD-1-OX40L-Exos or αCD3-αEGFR-PD-1-OX40L GEMINI-Exos to their targets. Briefly, cells (300,000 cells per tube) were stained with 100 μg mL⁻¹ of exosomes for 30 minutes at 4° C. Cells were washed three times with PBS containing 2% FBS and stained with an anti-HA antibody (2-2.2.14, from Thermo Fisher Scientific) for 30 minutes at 4° C. After three times of washing, cells were stained with an Alexa Fluor 488-labeled goat anti-mouse IgG H&L antibody (Catalog #A28175 from Thermo Fisher Scientific) for 30 minutes at 4° C. Positive controls were performed by staining the cells with respective antibodies (PE anti-PD-L1, clone: 29E.2A3; APC anti-PD-L2, clone: MIH18; PE/Cyanine7 anti-OX40, clone: Ber-ACT35; BioLegend) for 30 minutes at 4° C. Thereafter, cells were washed and resuspended in PBS containing 2% FBS, followed by analysis using a BD Fortessa X20 flow cytometer. Data were processed by FlowJo_V10 software (Tree Star Inc., Ashland, OR).

MDA-MB-468 cells (PD-L1⁻ PD-L2⁻ OX40⁻ CD3⁻ EGFR⁺) were used to examine co-expression of the HA-αCD3-αEGFR-PDGFR TMD and HA-PD-1-CD9-OX40L-6×His fusion proteins on the surface of the same exosome by flow cytometry. Briefly, cells (300,000 cells per tube) were stained with 100 μg mL⁻¹ of Native Exos, PD-1-OX40L-Exos, αCD3-αEGFR-Exos, or αCD3-αEGFR-PD-1-OX40L GEMINI-Exos for 30 minutes at 4° C. Cells were washed three times with PBS containing 2% FBS and stained with an anti-HA antibody (2-2.2.14, from Thermo Fisher Scientific) or an anti-6×His antibody (HIS.H8, from Thermo Fisher Scientific) for 30 minutes at 4° C. After three times of washing, cells were stained with an Alexa Fluor 488-labeled goat anti-mouse IgG H&L antibody (Catalog #A28175 from Thermo Fisher Scientific) for 30 minutes at 4° C. Thereafter, cells were washed and resuspended in PBS containing 2% FBS, followed by analysis using a BD Fortessa X20 flow cytometer. Data were processed by FlowJo_V10 software (Tree Star Inc., Ashland, OR).

Flow cytometry analysis of expression of EGFR, PD-L1, and PD-L2. HEK293 cells or three TNBC cells (MDA-MB-231, BT-20, MDA-MB-468) were treated with 100 U/mL IFN-7 or human PBMCs (PBMC:TNBC/HEK293=2:1) in the absence or presence of 20 ng/mL αCD3-αEGFR-Exos for 48 hours at 37° C. Treated and non-treated cells were stained with PE anti-PD-L1 (clone: 29E.2A3, BioLegend), APC anti-PD-L2 (clone: MIH18, BioLegend), or anti-EGFR (clone: AY13, BioLegend), followed by staining with the Alexa Fluor 488-labeled goat anti-mouse IgG H&L antibody (Catalog #A28175 from Thermo Fisher Scientific). Thereafter, cells were washed and resuspended in PBS containing 2% FBS, followed by analysis with a BD Fortessa X20 flow cytometer (BD Biosciences, CA). Data were processed by FlowJo_V10 software (Tree Star Inc., Ashland, OR).

T cell co-stimulation assays. Anti-human CD3 monoclonal Ab (clone: OKT3, BioLegend) was coated on surface-treated 96-well plates in 50 μL volume at a concentration of 10 μg mL⁻¹ at 37° C. for 3 hours, followed by washing three times with DPBS and additions of human PBMCs (1×10⁵ per well) in complete RPMI 1640 medium in the presence of various concentrations of PD-1-OX40L-Exos or native exosomes. After 48-hour incubation, cell culture supernatants were collected and assayed for the levels of interferon gamma (IFN-7) and interleukin-2 (IL-2) by ELISA kits (R&D Systems, Minneapolis, MN). Results are expressed as a mean±SD from one of at least three separate experiments.

To examine inhibitory effects of PD-L1 on PD-1-OX40L-Exos-induced T cell co-stimulation, the anti-CD3 monoclonal antibody (clone: OKT3, 10 μg mL⁻¹) together with PD-L1-6×His (10 μg ml⁻¹; GenScript, NJ) were coated on surface-treated 96-well plates at 37° C. for 3 hours, followed by washing three times with DPBS and additions of human PBMCs (1×10⁵ per well) in the presence of 10 μg mL⁻¹ PD-1-OX40L-Exos or native exosomes for 48 hours at 37° C. Cell culture supernatants were then collected and assayed for the levels of IL-2 by ELISA. Results are expressed as a mean±SD from one of at least three separate experiments.

To analyze stimulatory effects of PD-1-OX40L-Exos on αCD3-αEGFR-Exos-mediated T-cell activation, BT-20 cells and PBMCs were mixed at a ratio of 1:2 and incubated with αCD3-αEGFR-Exos (20 ng mL⁻¹) in the absence or presence of PD-1-OX40L-Exos (10 μg mL⁻¹) for 48 hours at 37° C. Cell culture supernatants were then collected and assayed for the levels of IL-2 by ELISA. Results are expressed as a mean±SD from one of at least three separate experiments.

To evaluate time-dependent T-cell activation, BT-20 and PBMCs were incubated at a ratio of 1:2 in the presence of native exosomes (10 μg mL⁻¹), PD-1-OX40L-Exos (10 μg mL⁻¹), αCD3-αEGFR-Exos (10 μg mL⁻¹), the combination of PD-1-OX40L-Exos (10 μg mL⁻¹) and αCD3-αEGFR-Exos (10 μg mL⁻¹), or αCD3-αEGFR-PD-1-OX40L GEMINI-Exos (10 μg mL⁻¹) for 24, 48, 72, and 96 hours. At each time point, cell culture supernatants were collected and assayed for the levels of IL-2 by ELISA. Results are expressed as a mean±SD from one of at least three separate experiments.

In vivo efficacy studies. Six to eight-week female NOD.Cg-Prkdc^{scid} I2rg^{tm1Wjl}/SzJ (NSG) mice were purchased from The Jackson Laboratory (Bar Harbor, ME). All animal experiments were approved by the Institutional Animal Care and Use Committee (IACUC) of the University of Southern California.

Mice (5 per group) were injected subcutaneously on the right hind limbs on day 0 with 5×10⁶ BT-20 cells in 0.1 mL of 50% matrigel (BD Biosciences). Human PBMCs were incubated in RPMI 1640 complete medium at a density of 2×10⁶ cells/mL and stimulated in flasks with immobilized anti-human CD3 antibody (clone: OKT3, BioLegend), soluble anti-CD28 antibody (2 μg mL⁻¹, clone: 28.2, BioLegend), and recombinant human IL-2 (rhIL-2) (40 IU mL⁻¹, BioLegend) for three days at 37° C. with 5% CO₂. Cells were then expanded in RPMI 1640 complete medium with 40 IU mU¹ rhIL-2. All human PBMCs purchased from HemaCare Corporation were from the same healthy donor. When tumor sizes reached 80-100 mm³, 12 days post tumor implantation, mice received two intraperitoneal injections of expanded human PBMCs (20×10⁶ cells per mouse) with a 6-day interval. One day following the first human PBMCs injection, mice were treated intravenously every other day for a total of six times with vehicle (PBS), native exosomes (10 mg/kg), PD-1-OX40L-Exos (10 mg/kg), αCD3-αEGFR-Exos (10 mg/kg), the combination of PD-1-OX40L-Exos (10 mg/kg) and αCD3-αEGFR-Exos (10 mg/kg), and αCD3-αEGFR-PD-1-OX40L GEMINI-Exos (10 mg/kg). Tumor volumes were measured three times weekly with a caliper and calculated as mm³=0.5×(length)×(width)². At the end of the study, mice were euthanized, and tumors, spleen, and blood were collected for lymphocyte isolation and analysis.

Lymphocyte isolation and analysis. The harvested blood samples were treated with red blood cell lysis buffer (BioLegend) by following manufacturer's instructions. Tumors and spleens were cut into small pieces and subjected to mechanical disruption and separation, followed by passing through 40 μm strainers and treatment with the red blood cell lysis buffer.

The resulting single-cell suspensions were stained for live and dead cells with live/dead-fixable Zombie Aqua (BioLegend), followed by cell surface marker staining with PerCP/Cyanine5.5 anti-human CD45 antibody (clone: 2D1, BioLegend), FITC anti-human CD3 antibody (clone: UCHT1, BioLegend), APC/Cyanine7 anti-human CD4 antibody (clone: OKT4, BioLegend), Pacific blue anti-human CD8 antibody (clone: RPA-T8, BioLegend), PE anti-human CD25 antibody (clone: M-A251, BioLegend), and PE/Dazzle 594 anti-human CD127 antibody (clone: A019D5, BioLegend). Prior to the intracellular staining for FoxP3 with PE/Dazzle 594 anti-human FoxP3 antibody (clone: 206D, BioLegend), cells were fixed with 4% paraformaldehyde (PFA) solution (Thermo Fisher Scientific) and permeabilized with 0.1% TritonX-100. Data acquisition was performed on a BD Fortessa X20 flow cytometer and results were analyzed with FlowJo. Total numbers of CD4⁺, CD8⁺, CD4⁺ CD25⁺ CD127⁻, and CD4⁺ CD25⁺ FoxP3⁺ cells were analyzed within CD45⁺ hematopoietic cell populations.

Immunohistofluorescence analysis. Immunostaining of collected tumors was performed on 7-mm cryosections by following standard protocols. Tumor tissues were fixed with 4% paraformaldehyde (PFA) for 10 minutes and then blocked with PBS containing 5% goat serum for 1 hour at room temperature. The tissue sections were then incubated with an anti-human CD3 antibody (clone: UCHT1, BioLegend) for 1 hour, stained with Alexa Fluor 488-conjugated anti-mouse IgG (H+L) secondary antibody (catalog #A28175 from Thermo Fisher Scientific) for 1 hour, followed by nuclei counterstaining with DAPI. Images were captured with a Leica SP8 confocal laser scanning microscope (Leica Microsystems Inc.) and processed using a LAS X software (Leica Microsystems Inc.). For quantification, three random, non-overlapping regions along the margin and interior of each tumor (n=3 mice/group) were imaged.

Alanine aminotransferase (ALT) activity assay. At the end of in vivo efficacy study, ALT activities in plasma samples were assayed. Collected mouse plasma samples (5 L) or a series of dilutions of standard solution (sodium pyruvate) were added to wells of clear 96-well plates, followed by additions of 25 L of ALT substrate solution (0.2 M alanine, 2 mM 2-oxoglutarate, pH 7.4) and incubation at 37° C. for 20 minutes. Next, 50 L of 2,4-DNPH (1 mM solution in 1 M HCl) was added and incubated at room temperature for 20 minutes. Then, 0.5 M sodium hydroxide was added, and absorbance was measured at 510 nm using a BioTek Synergy H1 Hybrid Multi-Mode Microplate reader (BioTek, VT). Amounts of generated pyruvate were calculated based on determined standard curves. ALT activity is reported as nmole/min/mL=unit/L, where one milliunit (mU) of ALT is defined as the amount of enzyme that generates 1.0 nmole of pyruvate per minute at 37° C.

Creatinine colorimetric assay. At the end of in vivo efficacy study, creatinine concentrations in plasma were determined by a colorimetric assay. Working solutions were prepared by mixing picric acid (38 mM) with sodium hydroxide (1.2 M) at 1:1 ratio. Mouse plasma samples were mixed with equal volume of TCA (10%) and centrifuged at 5000×g for 10 minutes. Collected supernatants and creatinine standards were added onto 96-well plates, followed by additions of working solution and incubation at room temperature for 45 minutes. Absorbance was measured at 500 nm using a BioTek Synergy H1 Hybrid Multi-Mode Microplate reader (BioTek, VT). Creatinine concentrations in mouse plasma samples were determined based on standard curves.

Statistical analysis. One-way ANOVA with Tukey post-hoc test was carried out for comparing multiple groups. Two-tailed unpaired t test was performed for comparison between two groups. A P<0.05 was considered statistically significant. Significance of finding was defined as: ns=not significant, P>0.05; *, P<0.05; **, P<0.01; ***, P<0.001 and ****, P<0.0001. Data are shown as mean±SD. All statistical analyses were calculated using GraphPad Prism (GraphPad Software, La Jolla, CA).

### Example 2. Pharmaceutical Dosage Forms

The following formulations illustrate representative pharmaceutical dosage forms that may be used for the therapeutic or prophylactic administration of a compound of a formula described herein, a compound specifically disclosed herein, or a pharmaceutically acceptable salt or solvate thereof (hereinafter referred to as ‘Composition X’):

| (i) Tablet 1 | mg/tablet |
|---|---|
| A vesicle as described herein | 100.0 |
| Lactose | 77.5 |
| Povidone | 15.0 |
| Croscarmellose sodium | 12.0 |
| Microcrystalline cellulose | 92.5 |
| Magnesium stearate | 3.0 |
| | 300.0 |
| (ii) Tablet 2 | mg/tablet |
| A vesicle as described herein | 20.0 |
| Microcrystalline cellulose | 410.0 |
| Starch | 50.0 |
| Sodium starch glycolate | 15.0 |
| Magnesium stearate | 5.0 |
| | 500.0 |

| (iii) Capsule | mg/capsule |
|---|---|
| A vesicle as described herein | 10.0 |
| Colloidal silicon dioxide | 1.5 |
| Lactose | 465.5 |
| Pregelatinized starch | 120.0 |
| Magnesium stearate | 3.0 |
| | 600.0 |

| (iv) Injection 1 (1 mg/mL) | mg/mL |
|---|---|
| A vesicle as described herein | 1.0 |
| (free acid form) | |
| Dibasic sodium phosphate | 12.0 |
| Monobasic sodium phosphate | 0.7 |
| Sodium chloride | 4.5 |
| 1.0N Sodium hydroxide solution | q.s. |
| (pH adjustment to 7.0-7.5) | |
| Water for injection | q.s. ad 1 mL |
| (v) Injection 2 (10 mg/mL) | mg/mL |
| A vesicle as described herein | 10.0 |
| (free acid form) | |
| Monobasic sodium phosphate | 0.3 |
| Dibasic sodium phosphate | 1.1 |
| Polyethylene glycol 400 | 200.0 |
| 0.1N Sodium hydroxide solution | q.s. |
| (pH adjustment to 7.0-7.5) | |
| Water for injection | q.s. ad 1 mL |

| (vi) Aerosol | mg/can |
|---|---|
| A vesicle as described herein | 20 |
| Oleic acid | 10 |
| Trichloromonofluoromethane | 5,000 |
| Dichlorodifluoromethane | 10,000 |
| Dichlorotetrafluoroethane | 5,000 |

| (vii) Topical Gel 1 | wt. % |
|---|---|
| A vesicle as described herein | 5% |
| Carbomer 934 | 1.25% |
| Triethanolamine | q.s. |
| (pH adjustment to 5-7) | |
| Methyl paraben | 0.2% |
| Purified water | q.s. to 100 g |
| (viii) Topical Gel 2 | wt. % |
| A vesicle as described herein | 5% |
| Methylcellulose | 2% |
| Methyl paraben | 0.2% |
| Propyl paraben | 0.02% |
| Purified water | q.s. to 100 g |
| (ix) Topical Ointment | wt. % |
| A vesicle as described herein | 5% |
| Propylene glycol | 1% |
| Anhydrous ointment base | 40% |
| Polysorbate 80 | 2% |
| Methyl paraben | 0.2% |
| Purified water | q.s. to 100 g |
| (x) Topical Cream 1 | wt. % |
| A vesicle as described herein | 5% |
| White bees wax | 10% |
| Liquid paraffin | 30% |
| Benzyl alcohol | 5% |
| Purified water | q.s. to 100 g |
| (xi) Topical Cream 2 | wt. % |
| A vesicle as described herein | 5% |
| Stearic acid | 10% |
| Glyceryl monostearate | 3% |
| Polyoxyethylene stearyl ether | 3% |
| Sorbitol | 5% |
| Isopropyl palmitate | 2% |
| Methyl Paraben | 0.2% |
| Purified water | q.s. to 100 g |

These formulations may be prepared by conventional procedures well known in the pharmaceutical art. It will be appreciated that the above pharmaceutical compositions may be varied according to well-known pharmaceutical techniques to accommodate differing amounts and types of active ingredient of a vesicle as described herein. Aerosol formulation (vi) may be used in conjunction with a standard, metered dose aerosol dispenser. Additionally, the specific ingredients and proportions are for illustrative purposes. Ingredients may be exchanged for suitable equivalents and proportions may be varied, according to the desired properties of the dosage form of interest.

### Example 3. Sequences

1.) Embodiment of a first fusion protein having the formula
HA Tag-αCD3VL-αCD3VH-αEGFRVH-αEGFRVL-Linker-PDGFR TMD:
(SEQ ID NO: 1)
YPYDVPDYAGAQPARSDIQMTQTTSSLSASLGDRVTISCRASQDIRNYLNWYQQKPDGT
VKLLIYYTSRLHSGVPSKFSGSGSGTDYSLTISNLEQEDIATYFCQQGNTLPWTFAGGTK
LEIKGGGSGGGSGGGSGGGSEVQLQQSGPELVKPGASMKISCKASGYSFTGYTMNWVK
QSHGKNLEWMGLINPYKGVSTYNQKFKDKATLTVDKSSSTAYMELLSLTSEDSAVYYC
ARSGYYGDSDWYFDVWGAGTTVTVSSGGGGSGGGGSGGGGSQVQLKQSGPGLVQPS
QSLSITCTVSGFSLTNYGVHWVRQSPGKGLEWLGVIWSGGNTDYNTPFTSRLSINKDNS
KSQVFFKMNSLQSNDTAIYYCARALTYYDYEFAYWGQGTLVTVSAGGGGSGGGGSGG
GGSDILLTQSPVILSVSPGERVSFSCRASQSIGTNIHWYQQRTNGSPRLLIKYASESISGIPS
RFSGSGSGTDFTLSINSVESEDIADYYCQQNNNWPTTFGAGTKLELKGGGGSVDAVGQD
TQEVIVVPHSLPFKVVVISAILALVVLTIISLIILIMLWQKKPR
2. DNA sequence of first fusion protein having the formula
HA Tag-αCD3VL-αCD3VH-αEGFRVH-αEGFRVL-Linker-PDGFR TMD:
(SEQ ID NO: 2)
atccatatgatgttccagattatgctggggcccagccggccagatctgatatccagatgacacagacaacctcaagtcttagtgcatcactgg
gagatcgtgtgactataagctgccgcgcatcacaggacattcgcaattatctgaattggtatcaacagaagcctgatggcaccgtgaaacttc
tgatctattacaccagtcgtctgcatagcggtgttccgagcaaattttcaggctcagggtcaggaaccgattattcactgacgattagtaattta
gaacaagaagatattgcaacctatttctgtcaacagggtaataccctgccgtggacctttgcaggtggtaccaaactggaaattaaaggaggt
ggcagtggagggggaagcggcggcggttcaggaggcggttctgaggtccagttacagcagagcggtccggaactggttaaaccgggtg
caagcatgaaaattagctgtaaagcaagcggttatagctttaccggttataccatgaattgggttaaacagagccatggtaaaaatctggaatg
gatgggtctgattaatccgtataaaggtgttagcacctataatcagaaatttaaagataaagcaaccctgaccgttgataaaagcagcagcac
cgcatatatggaactgctgagcctgaccagcgaagatagcgccgtttactattgcgcacgcagcggttattatggtgatagcgattggtatttt
gatgtttggggtgcaggtaccaccgttaccgttagcagcggaggtggcggaagtggaggaggtggctctggcggtggaggaagccaggt
gcagctgaagcagtctggccctggactggtgcagcctagccagagcctgagcatcacctgtaccgtgtccggcttcagcctgaccaactac
ggcgtgcactgggtgcgacagagccctggcaaaggcctggaatggctgggagtgatttggagcggcggcaacaccgactacaacaccc
ccttcaccagcagactgtccatcaacaaggacaacagcaagagccaggtgttcttcaagatgaacagcctgcagagcaacgacaccgcc
atctactactgcgctagagccctgacctactatgactacgagttcgcctactggggccagggcacactcgtgacagtgtctgccggcggag
gtggatctggaggcggtggcagcggtggaggcggatctgacatcctgctgacccagagccccgtgatcctgtccgtgtctcctggcgaga
gagtgtccttcagctgcagagccagccagagcatcggcaccaacatccactggtatcagcagaggaccaacggcagccccagactgctg
attaagtacgccagcgagtccatcagcggcatccccagcagattcagcggcagcggctctggcaccgacttcaccctgagcatcaacagc
gtggaaagcgaggatatcgccgactactactgccagcagaacaacaactggcccaccaccttcggcgctggcaccaagctggaactgaa
gggcgggggcggaagcgtcgacgaacaaaaactcatctcagaagaggatctgaatgctgtgggccaggacacgcaggaggtcatcgtg
gtgccacactccttgccctttaaggtggtggtgatctcagccatcctggccctggtggtgctcaccatcatctcccttatcatcctcatcatgcttt
ggcagaagaagccacgttag
3.) Embodiment of a second fusion protein having the formula
HA Tag-PD1-CD9-OX40L-6x His Tag:
(SEQ ID NO: 3)
YPYDVPDYAGAQPARSFLDSPDRPWNPPTFSPALLVVTEGDNATFTCSFSNTSESFVLN
WYRMSPSNQTDKLAAFPEDRSQPGQDCRFRVTQLPNGRDFHMSVVRARRNDSGTYLC
GAISLAPKAQIKESLRAELRVTERRAEVPTAHPSPSPRPAGQFQTLVVGVVGGLLGSLVL
LVWVLAVICSRAARGTIGARRTGQPLKEDPSAVPVFSVDYGELDFQWREKTPEPPVPCV
PEQTEYATIVFPSGMGTSSPARRGSADGPRSAQPLRPEDGHCSWPLASGGGGSPVKGGT
KCIKYLLFGFNFIFWLAGIAVLAIGLWLRFDSQTKSIFEQETNNNNSSFYTGVYILIGAGA
LMMLVGFLGCCGAVQESQCMLGLFFGFLLVIFAIEIAAAIWGYSHKDEVIKEVQEFYKD
TYNKLKTKDEPQRETLKAIHYALNCCGLAGGVEQFISDICPKKDVLETFTVKSCPDAIKE
VFDNKFHIIGAVGIGIAVVMIFGMIFSMILCCAIRRNREMVGGGGSGGGGSVDERVQPLE
ENVGNAARPRFERNKLLLVASVIQGLGLLLCFTYICLHFSALQVSHRYPRIQSIKVQFTEY
KKEKGFILTSQKEDEIMKVQNNSVIINCDGFYLISLKGYFSQEVNISLHYQKDEEPLFQLK
KVRSVNSLMVASLTYKDKVYLNVTTDNTSLDDFHVNGGELILIHQNPGEFCVLHHHHH
H
4. DNA sequence of a second fusion protein having the formula
HA Tag-PD1-CD9-OX40L-6x His Tag:
(SEQ ID NO: 4)
tatccatatgatgttccagattatgctggggcccagccggccagatctttcttagactccccagacaggccctggaacccccccaccttctcc
ccagccctgctcgtggtgaccgaaggggacaacgccaccttcacctgcagcttctccaacacatcggagagcttcgtgctaaactggtacc
gcatgagccccagcaaccagacggacaagctggccgctttccccgaggaccgcagccagcccggccaggactgccgcttccgtgtcac
acaactgcccaacgggcgtgacttccacatgagcgtggtcagggcccggcgcaatgacagcggcacctacctctgtggggccatctccct
ggcccccaaggcgcagatcaaagagagcctgcgggcagagctcagggtgacagagagaagggcagaagtgcccacagcccacccca
gcccctcacccaggccagccggccagttccaaaccctggtggttggtgtcgtgggcggcctgctgggcagcctggtgctgctagtctggg
tcctggccgtcatctgctcccgggccgcacgagggacaataggagccaggcgcaccggccagcccctgaaggaggacccctcagccgt
gcctgtgttctctgtggactatggggagctggatttccagtggcgagagaagaccccggagccccccgtgccctgtgtccctgagcagacg
gagtatgccaccattgtctttcctagcggaatgggcacctcatcccccgcccgcaggggctcagccgacggccctcggagtgcccagcca
ctgaggcctgaggatggacactgctcttggcccctcgctagcggaggcggtggtagtccggtcaaaggaggcaccaagtgcatcaaatac
ctgctgttcggatttaacttcatcttctggcttgccgggattgctgtccttgccattggactatggctccgattcgactctcagaccaagagcatct
tcgagcaagaaactaataataataattccagcttctacacaggagtctatattctgatcggagccggcgccctcatgatgctggtgggcttcct
gggctgctgcggggctgtgcaggagtcccagtgcatgctgggactgttcttcggcttcctcttggtgatattcgccattgaaatagctgcggc
catctggggatattcccacaaggatgaggtgattaaggaagtccaggagttttacaaggacacctacaacaagctgaaaaccaaggatgag
ccccagcgggaaacgctgaaagccatccactatgcgttgaactgctgtggtttggctgggggcgtggaacagtttatctcagacatctgccc
caagaaggacgtactcgaaaccttcaccgtgaagtcctgtcctgatgccatcaaagaggtcttcgacaataaattccacatcatcggcgcag
tgggcatcggcattgccgtggtcatgatatttggcatgatcttcagtatgatcttgtgctgtgctatccgcaggaaccgcgagatggtcggagg
cggtggtagcggtggaggtgggtccgtcgacgaaagggtccaacccctggaagagaatgtgggaaatgcagccaggccaagattcgag
aggaacaagctattgctggtggcctctgtaattcagggactggggctgctcctgtgcttcacctacatctgcctgcacttctctgctcttcaggt
atcacatcggtatcctcgaatacaaagtatcaaagtacaatttaccgaatataagaaggagaaaggtttcatcctcacttcccaaaaggaggat
gaaatcatgaaggtgcagaacaactcagtcatcatcaactgtgatgggttttatctcatctccctgaagggctacttctcccaggaagtcaaca
ttagccttcattaccagaaggatgaggagcccctcttccaactgaagaaggtcaggtctgtcaactccttgatggtggcctctctgacttacaa
agacaaagtctacttgaatgtgaccactgacaatacctccctggatgacttccatgtgaatggcggagaactgattcttatccatcaaaatcct
ggtgagttctgtgtccttcaccatcaccaccaccattaa
5.) αCD3VL-Linker-αCD3VH (i.e., scFv):
(SEQ ID NO: 5)
DIQMTQTTSSLSASLGDRVTISCRASQDIRNYLNWYQQKPDGTVKLLIYYTSRLHSGVPS
KFSGSGSGTDYSLTISNLEQEDIATYFCQQGNTLPWTFAGGTKLEIKGGGSGGGSGGGSG
GGSEVQLQQSGPELVKPGASMKISCKASGYSFTGYTMNWVKQSHGKNLEWMGLINPY
KGVSTYNQKFKDKATLTVDKSSSTAYMELLSLTSEDSAVYYCARSGYYGDSDWYFDV
WGAGTTVTVSS
6.) αEGFRVH-Linker-αEGFRVL (i.e., scFv):
(SEQ ID NO: 6)
QVQLKQSGPGLVQPSQSLSITCTVSGFSLTNYGVHWVRQSPGKGLEWLGVIWSGGNTD
YNTPFTSRLSINKDNSKSQVFFKMNSLQSNDTAIYYCARALTYYDYEFAYWGQGTLVT
VSAGGGGSGGGGSGGGGSDILLTQSPVILSVSPGERVSFSCRASQSIGTNIHWYQQRTNG
SPRLLIKYASESISGIPSRFSGSGSGTDFTLSINSVESEDIADYYCQQNNNWPTTFGAGTKL
ELK
7.) PDGFR TMD:
(SEQ ID NO: 7)
AVGQDTQEVIVVPHSLPFKVVVISAILALVVLTIISLIILIMLWQKKPR
8.) PD1:
(SEQ ID NO: 8)
MQIPQAPWPVVWAVLQLGWRPGWFLDSPDRPWNPPTFSPALLVVTEGDNATFTCSFSN
TSESFVLNWYRMSPSNQTDKLAAFPEDRSQPGQDCRFRVTQLPNGRDFHMSVVRARRN
DSGTYLCGAISLAPKAQIKESLRAELRVTERRAEVPTAHPSPSPRPAGQFQTLVVGVVGG
LLGSLVLLVWVLAVICSRAARGTIGARRTGQPLKEDPSAVPVFSVDYGELDFQWREKTP
EPPVPCVPEQTEYATIVFPSGMGTSSPARRGSADGPRSAQPLRPEDGHCSWPL
9. PD-L1:
(SEQ ID NO: 9)
MRIFAVFIFMTYWHLLNAPYNKINQRILVVDPVTSEHELTCQAEGYPKAEVIWTSSD
HQVLSGKTTTTNSKREEKLFNVTSTLRINTTTNEIFYCTFRRLDPEENHTAELVIPELPL
AHPPNERTHLVILGAILLCLGVALTFIFRLRKGRMMDVKKCGIQDTNSKKQSDTHLE
ET
10. PD-L2:
(SEQ ID NO: 10)
MIFLLLMLSLELQLHQIAALFTVTVPKELYIIEHGSNVTLECNFDTGSHVNLGAITASLQK
VENDTSPHRERATLLEEQLPLGKASFHIPQVQVRDEGQYQCIIIYGVAWDYKYLTLKVK
ASYRKINTHILKVPETDEVELTCQATGYPLAEVSWPNVSVPANTSHSRTPEGLYQVTSVL
RLKPPPGRNFSCVFWNTHVRELTLASIDLQSQMEPRTHPTWLLHIFIPFCIIAFIFIATVIAL
RKQLCQKLYSSKDTTKRPVTTTKREVNSAI
11.) CD9:
(SEQ ID NO: 11)
MPVKGGTKCIKYLLFGFNFIFWLAGIAVLAIGLWLRFDSQTKSIFEQETNNNNSSFYTGV
YILIGAGALMMLVGFLGCCGAVQESQCMLGLFFGFLLVIFAIEIAAAIWGYSHKDEVIKE
VQEFYKDTYNKLKTKDEPQRETLKAIHYALNCCGLAGGVEQFISDICPKKDVLETFTVK
SCPDAIKEVFDNKFHIIGAVGIGIAVVMIFGMIFSMILCCAIRRNREMV
12. EGFR:
(SEQ ID NO: 12)
MRPSGTAGAALLALLAALCPASRALEEKKVCQGTSNKLTQLGTFEDHFLSLQRMFN
NCEVVLGNLEITYVORNYDLSFLKTIQEVAGYVLIALNTVERIPLENLQIIRGNMYYE
NSYALAVLSNYDANKTGLKELPMRNLQGQKCDPSCPNGSCWGAGEENCOKLTKIIC
AQQCSGRCRGKSPSDCCHNQCAAGCTGPRESDCLVCRKFRDEATCKDTCPPLMLYN
PTTYQMDVNPEGKYSFGATCVKKCPRNYVVTDHGSCVRACGADSYEMEEDGVRKC
KKCEGPCRKVCNGIGIGEFKDSLSINATNIKHFKNCTSISGDLHILPVAFRGDSFTHTPP
LDPQELDILKTVKEITGFLLIQAWPENRTDLHAFENLEIIRGRTKQHGQFSLAVVSLNI
TSLGLRSLKEISDGDVIISGNKNLCYANTINWKKLFGTSGQKTKIISNRGENSCKATGQ
VCHALCSPEGCWGPEPRDCVSCRNVSRGRECVDKCNLLEGEPREFVENSECIQCHPE
CLPQAMNITCTGRGPDNCIQCAHYIDGPHCVKTCPAGVMGENNTLVWKYADAGHV
CHLCHPNCTYGCTGPGLEGCPTNGPKIPSIATGMVGALLLLLVVALGIGLFMRRRHIV
RKRTLRRLLQERELVEPLTPSGEAPNQALLRILKETEFKKIKVLGSGAFGTVYKGLWI
PEGEKVKIPVAIKELREATSPKANKEILDEAYVMASVDNPHVCRLLGICLTSTVQLIT
QLMPFGCLLDYVREHKDNIGSQYLLNWCVQIAKGMNYLEDRRLVHRDLAARNVLV
KTPQHVKITDFGLAKLLGAEEKEYHAEGGKVPIKWMALESILHRIYTHQSDVWSYG
VTVWELMTFGSKPYDGIPASEISSILEKGERLPQPPICTIDVYMIMVKCWMIDADSRP
KFRELIIEFSKMARDPQRYLVIQGDERMHLPSPTDSNFYRALMDEEDMDDVVDADE
YLIPQOGFFSSPSTSRTPLLSSLSATSNNSTVACIDRNGLQSCPIKEDSFLORYSSDPTG
ALTEDSIDDTFLPVPGEWLVWKQSCSSTSSTHSAAASLQCPSQVLPPASPEGETVADL
QTQ
13. OX40:
(SEQ ID NO: 13)
MCVGARRLGRGPCAALLLLGLGLSTVTGLHCVGDTYPSNDRCCHECRPGNGMVSR
CSRSQNTVCRPCGPGFYNDVVSSKPCKPCTWCNLRSGSERKQLCTATQDTVCRCRA
GTQPLDSYKPGVDCAPCPPGHFSPGDNQACKPWTNCTLAGKHTLQPASNSSDAICED
RDPPATQPQETQGPPARPITVQPTEAWPRTSQGPSTRPVEVPGGRAVAAILGLGLVLG
LLGPLAILLALYLLRRDORLPPDAHKPPGGGSFRTPIQEEQADAHSTLAKI
14. OX40L:
(SEQ ID NO: 14)
MERVQPLEENVGNAARPRFERNKLLLVASVIQGLGLLLCFTYICLHFSALQVSHRYPRIQ
SIKVQFTEYKKEKGFILTSQKEDEIMKVQNNSVIINCDGFYLISLKGYFSQEVNISLHYQK
DEEPLFQLKKVRSVNSLMVASLTYKDKVYLNVTTDNTSLDDFHVNGGELILIHQNPGEF
CVL
15. CD3 delta:
(SEQ ID NO: 15)
MEHSTFLSGLVLATLLSQVSPFKIPIEELEDRVFVNCNTSITWVEGTVGTLLSDITRLD
LGKRILDPRGIYRCNGTDIYKDKESTVOVHYRMCQSCVELDPATVAGIIVTDVIATLL
LALGVFCFAGHETGRLSGAADTQALLRNDQVYQPLRDRDDAQYSHLGGNWARNK
16. CD3 epsilon:
(SEQ ID NO: 16)
MQSGTHWRVLGLCLLSVGVWGQDGNEEMGGITQTPYKVSISGTTVILTCPQYPGSEI
LWOHNDKNIGGDEDDKNIGSDEDHLSLKEFSELEQSGYYVCYPRGSKPEDANFYLY
LRARVCENCMEMDVMSVATIVIVDICITGGLLLLVYYWSKNRKAKAKPVTRGAGAG
GRORGONKERPPPVPNPDYEPIRKGQRDLYSGLNQRRI
17. CD3 gamma:
(SEQ ID NO: 17)
MEQGKGLAVLILAIILLOGTLAQSIKGNHLVKVYDYQEDGSVLLTCDAEAKNITWFK
DGKMIGFLTEDKKKWNLGSNAKDPRGMYQCKGSQNKSKPLQVYYRIVICONCIELNA
ATISGFLFAEIVSIFVLAVGVYFIAGQDGVRQSRASDKQTLLPNDOLYQPLKDREDDO
YSHLQGNQLRRN
18. CD4:
(SEQ ID NO: 18)
MNRGVPFREILLLVLQLALLPAATQGKKVVLGKKGDTVELTCTASQKKSIQFHWKNS
NQIKILGNQGSFLTKGPSKLNDRADSRRSLWDQGNFPLIIKNLKIEDSDTYICEVEDQK
EEVQLLVFGLTANSDTHLLQGQSLTLTLESPPGSSPSVQCRSPRGKNIQGGKTLSVSQ
LELQDSGTWTCTVLQNQKKVEFKIDIVVLAFQKASSIVYKKEGEQVEFSFPLAFTVEK
LTGSGELWWQAERASSSKSWITFDLKNKEVSVKRVTQDPKLQMGKKLPLHLTLPQA
LPQYAGSGNLTLALEAKTGKLHQEVNLVVMRATQLQKNLTCEVWGPTSPKLMLSL
KLENKEAKVSKREKAVWVLNPEAGMWQCLLSDSGQVLLESNIKVLPTWSTPVQPM
ALIVLGGVAGLLLFIGLGIFFCVRCRHRRRQAERMSQIKRLLSEKKTCQCPHRFQKTC
SPI
19. CD8 alpha:
(SEQ ID NO: 19)
MALPVTALLLPLALLLHAARPSQFRVSPLDRTWNLGETVELKCQVLLSNPTSGCSWL
FQPRGAAASPTFLLYLSQNKPKAAEGLDTQRFSGKRLGDTFVLTLSDFRRENEGYYF
CSALSNSIMYFSHFVPVFLPAKPTTTPAPRPPTPAPTIASQPLSLRPEACRPAAGGAVH
TRGLDFACDIYIWAPLAGTCGVLLLSLVITLYCNHRNRRRVCKCPRPVVKSGDKPSLSA
RYV
20. CD8 beta:
(SEQ ID NO: 20)
MRPRLWLLLAAQLTVLHGNSVLQQTPAYIKVQTNKMVMLSCEAKISLSNMRIYWLR
QRQAPSSDSHHEFLALWDSAKGTIHGEEVEQEKIAVFRDASRFILNLTSVKPEDSGIY
FCMIVGSPELTFGKGTQLSVVDFLPTTAQPTKKSTLKKRVCRLPRPETQKGLKGKVY
QEPLSPNACMDTTAILQPHRSCLTHGS
21. CD16 alpha:
(SEQ ID NO: 21)
MWQLLLPTALLLLVSAGMRTEDLPKAVVFLEPQWYRVLEKDSVTLKCQGAYSPEDNST
QWFHNESLISSQASSYFIDAATVDDSGEYRCQTNLSTLSDPVQLEVHIGWLLLQAPRWV
FKEEDPIHLRCHSWKNTALHKVTYLQNGKGRKYFHHNSDFYIPKATLKDSGSYFCRGLF
GSKNVSSETVNITITQGLAVSTISSFFPPGYQVSFCLVMVLLFAVDTGLYFSVKTNIRSSTR
DWKDHKFKWRKDPQDK
22. CD16 beta:
(SEQ ID NO: 22)
MWQLLLPTALLLLVSAGMRTEDLPKAVVFLEPQWYSVLEKDSVTLKCQGAYSPEDNST
QWFHNENLISSQASSYFIDAATVNDSGEYRCQTNLSTLSDPVQLEVHIGWLLLQAPRWV
FKEEDPIHLRCHSWKNTALHKVTYLQNGKDRKYFHHNSDFHIPKATLKDSGSYFCRGL
VGSKNVSSETVNITITQGLAVSTISSFSPPGYQVSFCLVMVLLFAVDTGLYFSVKTNI
23. ICOS:
(SEQ ID NO: 23)
MKSGLWYFFLFCLRIKVLTGEINGSANYEMFIFEINGGVQILCKYPDIVQQFKMQLLK
GGQILCDLTKTKGSGNTVSIKSLKFCHSQLSNNSVSFFLYNLDHSHANYYFCNLSIFD
PPPFKVTLTGGYLHIYESQLCCQLKFWLPIGCAAFVVVCILGCILICWLTKKKYSSSVH
DPNGEYMFMRAVNTAKKSRLTDVTL
24. CD137:
(SEQ ID NO: 24)
MGNSCYNIVATLLLVLNFERTRSLQDPCSNCPAGTFCDNNRNQICSPCPPNSFSSAGGQR
TCDICRQCKGVFRTRKECSSTSNAECDCTPGFHCLGAGCSMCEQDCKQGQELTKKGCK
DCCFGTFNDQKRGICRPWTNCSLDGKSVLVNGTKERDVVCGPSPADLSPGASSVTPPAP
AREPGHSPQIISFFLALTSTALLFLLFFLTLRFSVVKRGRKKLLYIFKQPFMRPVQTTQEED
GCSCRFPEEEEGGCEL
25. LAG3:
(SEQ ID NO: 25)
MWEAQFLGLLFLQPLWVAPVKPLQPGAEVPVVWAQEGAPAQLPCSPTIPLQDLSLL
RRAGVTWQHQPDSGPPAAAPGHPLAPGPHPAAPSSWGPRPRRYTVLSVGPGGLRSG
RLPLQPRVQLDERGRQRGDFSLWLRPARRADAGEYRAAVHLRDRALSCRLRLRLGQ
ASMTASPPGSLRASDWVILNCSFSRPDRPASVHWFRNRGQGRVPVRESPHEIHLAESF
LFLPQVSPMDSGPWGCILTYRDGFNVSIMYNLTVLGLEPPTPLTVYAGAGSRVGLPC
RLPAGVGTRSFLTAKWTPPGGGPDLLVTGDNGDFTLRLEDVSQAQAGTYTCHIHLQ
EQQLNATVTLAIITVTPKSFGSPGSLGKLLCEVTPVSGQERFVWSSLDTPSQRSFSGP
WLEAQEAQLLSQPWQCQLYQGERLLGAAVYFTELSSPGAQRSGRAPGALPAGHLLL
FLILGVLSLLLLVTGAFGFHLWRRQWRPRRFSALEQGIHPPQAQSKIEELEQEPEPEPE
PEPEPEPEPEPEQL
26. CTLA4:
(SEQ ID NO: 26)
MACLGFQRHKAQLNLATRTWPCTLLFFLLFIPVFCKAMHVAQPAVVLASSRGIASFV
CEYASPGKATEVRVTVLRQADSQVTEVCAATYMMGNELTFLDDSICTGTSSGNQVN
LTIQGLRAMDTGLYICKVELMYPPPYYLGIGNGTQIYVIAKEKKPSYNRGLCENAPN
RARM
27. CLL-1:
(SEQ ID NO: 27)
MWIDFFTYSSMSEEVTYADLQFQNSSEMEKIPEIGKFGEKAPPAPSHVWRPAALFLTL
LCLLLLIGLGVLASMEHVTLKIEMKKMNKLQNISEELQRNISLQLMSNMNISNKIRNL
STTLQTIATKLCRELYSKEQEHKCKPCPRRWIWHKDSCYFLSDDVQTWQESKMACA
AQNASLLKINNKNALEFIKSQSRSYDYWLGLSPEEDSTRGMRVDNIINSSAWVIRNAP
DLNNMYCGYINRLYVQYYHCTYKKRMICEKMANPVQLGSTYFREA
28. HER2:
(SEQ ID NO: 28)
MKLRLPASPETHLDMLRHLYQGCQVVQGNLELTYLPTNASLSFLQDIQEVQGYVLIA
HNQVRQVPLQRLRIVRGTQLFEDNYALAVLDNGDPLNNTTPVTGASPGGLRELQLRS
LTEILKGGVLIQRNPQLCYQDTILWKDIFHKNNQLALTLIDTNRSRACHPCSPMCKGS
RCWGESSEDCQSLTRTVCAGGCARCKGPLPTDCCHEQCAAGCTGPKHSDCLACLHF
NHSGICELHCPALVTYNTDTFESMPNPEGRYTFGASCVTACPYNYLSTDVGSCTLVC
PLHNQEVTAEDGTQRCEKCSKPCARVCYGLGMEHLREVRAVTSANIQEFAGCKKIF
GSLAFLPESFDGDPASNTAPLQPEQLQVFETLEEITGYLYISAWPDSLPDLSVFQNLQV
IRGRILHNGAYSLTLQGLGISWLGLRSLRELGSGLALIHENTHLCFVHTVPWDQLFRN
PHQALLHTANRPEDECVGEGLACHQLCARGHCWGPGPTQCVNCSQFLRGQECVEE
CRVLQGLPREYVNARHCLPCHPECQPQNGSVTCFGPEADQCVACAHYKDPPFCVAR
CPSGVKPDLSYMPIWKFPDEEGACQPCPINCTHSCVDLDDKGCPAEQRASPLTSIISA
VVGILLVVVLGVVFGILIKRRQQKIRKYTMRRLLQETELVEPLTPSGAMPNQAQMRIL
KETELRKVKVLGSGAFGTVYKGIWIPDGENVKIPVAIKVLRENTSPKANKEILDEAYV
MAGVGSPYVSRLLGICLTSTVQLVTQLMPYGCLLDHVRENRGRLGSQDLLNWCMQI
AKGMSYLEDVRLVHRDLAARNVLVKSPNHVKITDFGLARLLDIDETEYHADGGKVP
IKWMALESILRRRFTHQSDVWSYGVTVWELMTFGAKPYDGIPAREIPDLLEKGERLP
QPPICTIDVYMIMVKCWMIDSECRPRFRELVSEFSRMARDPQRFVVIQNEDLGPASPL
DSTFYRSLLEDDDMGDLVDAEEYLVPQQGFFCPDPAPGAGGMVHHRHRSSSTRSGG
GDLTLGLEPSEEEAPRSPLAPSEGAGSDVFDGDLGMGAAKGLQSLPTHDPSPLQRYS
EDPTVPLPSETDGYVAPLTCSPQPEYVNQPDVRPQPPSPREGPLPAARPAGATLERPK
TLSPGKNGVVKDVFAFGGAVENPEYLTPQGGAAPQPHPPPAFSPAFDNLYYWDQDP
PERGAPPSTFKGTPTAENPEYLGLDVPV
29. CD33:
(SEQ ID NO: 29)
MPLLLLLPLLWADLTHRPKILIPGTLEPGHSKNLTCSVSWACEQGTPPIFSWLSAAPTSLG
PRTTHSSVLIITPRPQDHGTNLTCQVKFAGAGVTTERTIQLNVTYVPQNPTTGIFPGDGSK
QETRAGVVHGAIGGAGVTALLALCLCLIFFIVKTHRRKAARTAVGRNDTHPTTGSASPK
HQKKSKLHGPTETSSCSGAAPTVEMDEELHYASLNFHGMNPSKDTSTEYSEVRTQ
30: CD38:
(SEQ ID NO: 30)
MANCEFSPVSGDKPCCRLSRRAQLCLGVSILVLILVVVLAVVVPRWRQQWSGPGTTKRF
PETVLARCVKYTEIHPEMRHVDCQSVWDAFKGAFISKHPCNITEEDYQPLMKLGTQTVP
CNKILLWSRIKDLAHQFTQVQRDMFTLEDTLLGYLADDLTWCGEFNTSKINYQSCPDW
RKDCSNNPVSVFWKTVSRRFAEAACDVVHVMLNGSRSKIFDKNSTFGSVEVHNLQPEK
VQTLEAWVIHGGREDSRDLCQDPTIKELESIISKRNIQFSCKNIYRPDKFLQCVKNPEDSS
CTSEI
31. CD123:
(SEQ ID NO: 31)
MVLLWLTLLLIALPCLLQTKEGGKPWAGAENLTCWIHDVDFLSCSWAVGPGAPADVQ
YDLYLNVANRRQQYECLHYKTDAQGTRIGCRFDDISRLSSGSQSSHILVRGRSAAFGIPC
TDKFVVFSQIEILTPPNMTAKCNKTHSFMHWKMRSHFNRKFRYELQIQKRMQPVITEQV
RDRTSFQLLNPGTYTVQIRARERVYEFLSAWSTPQRFECDQEEGANTRAWRTSLLIALG
TLLALVCVFVICRRYLVMQRLFPRIPHMKDPIGDSFQNDKLVVWEAGKAGLEECLVTEV
QVVQKT
32. TIM3:
(SEQ ID NO: 32)
MFSHLPFDCVLLLLLLLLTRSSEVEYRAEVGQNAYLPCFYTPAAPGNLVPVCWGKG
ACPVFECGNVVLRTDERDVNYWTSRYWLNGDFRKGDVSLTIENVTLADSGIYCCRI
QIPGIMNDEKFNLKLVIKPAKVTPAPTRQRDFTAAFPRMLTTRGHGPAETQTLGSLPD
INLTQISTLANELRDSRLANDLRDSGATIRIGIYIGAGICAGLALALIFGALIFKWYSHS
KEKIQNLSLISLANLPPSGLANAVAEGIRSEENIYTIEENVYEVEEPNEYYCYVSSRQQ
PSQPLGCRFAMP
33. Linker:
(SEQ ID NO: 33)
(GGGGS)₂
34. Linker:
(SEQ ID NO: 34)
(GGGS)n
35. Linker:
(SEQ ID NO: 35)
(GGGS)₄
36. Linker:
(SEQ ID NO: 36)
(GGGGS)n
37. Linker:
(SEQ ID NO: 37)
(GGGGS)₃
38.) Myc Tag:
(SEQ ID NO: 38)
EQKLISEEDL
39. 6X Histidine Tag:
(SEQ ID NO: 39)
HHHHHH
40. Hemagglutinin (HA) Tag:
(SEQ ID NO: 40)
YPYDVPDYA

While specific embodiments have been described above with reference to the disclosed embodiments and examples, such embodiments are only illustrative and do not limit the scope of the invention. Changes and modifications can be made in accordance with ordinary skill in the art without departing from the invention in its broader aspects as defined in the following claims.

All publications, patents, and patent documents are incorporated by reference herein, as though individually incorporated by reference, and in particular, U.S. Patent Pub. No. US 2020/0405640, International Patent Pub. No. WO 2020/205579, and Cheng et al., Mol Ther. 2022 Jun. 22; S1525-0016(22)00373-2. doi: 10.1016/j.ymthe.2022.06.013. No limitations inconsistent with this disclosure are to be understood therefrom. The invention has been described with reference to various specific and preferred embodiments and techniques. However, it should be understood that many variations and modifications may be made while remaining within the spirit and scope of the invention.

1. An engineered extracellular vesicle comprising:
a first fusion protein comprising a formula A-B-C, wherein A is a first antibody moiety, B is a second antibody moiety, and C is a first exosomal protein transmembrane domain; and
a second fusion protein comprising the formula D-E-F, wherein D is a first protein binding moiety, E is a second exosomal membrane protein transmembrane domain, and F is a second protein binding moiety;
wherein both the first fusion protein and the second fusion protein are displayed on a surface of the engineered extracellular vesicle, and the first antibody moiety and the second antibody moiety separately bind to a first immune cell surface-marker protein and a first cancer cell surface-marker protein, and the first protein binding moiety and the second protein binding moiety separately bind to a second cancer cell surface-marker protein and a second immune cell surface-marker protein.

2. The engineered extracellular vesicle of claim 1 wherein the extracellular vesicle comprises one or more of an exosome, a liposome, a microvesicle, and an apoptotic body.

3. The engineered extracellular vesicle of claim 1 wherein the first antibody moiety and the second antibody moiety are a single chain variable fragment (scFv), a single domain antibody, a bispecific antibody, or a multispecific antibody.

4. The engineered extracellular vesicle of claim 1 wherein the first immune cell surface-marker protein and the second immune cell surface-marker protein is CD3, OX40, CD2, CD4, CD5, CD7, CD8, CD14, CD15, CD16, CD24, CD25, CD27, CD28, CD30, CD31, CD38, CD40L, CD45, CD56, CD68, CD91, CD114, CD163, CD206, LFA1, PD-1, ICOS, BTLA, KIR, CD137, LAG3, CTLA4, or a T-cell Receptor, wherein the first immune cell surface-marker protein and the second immune cell surface-marker protein are not the same.

5. The engineered extracellular vesicle of claim 1 wherein the first cancer cell surface-marker protein and the second cancer cell surface-marker protein is EGFR, CLL-1, HER2, HER3, CD33, CD34, CD38, CD123, TIM3, CD25, CD32, CD96, PD-L1, or PD-L2, wherein the first cancer cell surface-marker protein and the second cancer cell surface-marker protein are not the same.

6. The engineered extracellular vesicle of claim 1 wherein the first cancer cell surface-marker protein is epidermal growth factor receptor (EGFR) and the first immune cell surface-marker protein is CD3.

7. The engineered extracellular vesicle of claim 1 wherein the first protein binding moiety is a type I membrane protein and the second protein binding moiety is a type II membrane protein.

8. The engineered extracellular vesicle of claim 7 wherein the type I membrane protein is PD-1, and the type II membrane protein is OX40L, wherein the PD-1 binds to PD-L1/L2, and the OX40L binds to OX40, and wherein the PD-L1/L2 and the OX40 are disposed on a surface of a tumor cell and an immune cell, respectively.

9. The engineered extracellular vesicle of claim 7 wherein the type I membrane protein is LAG3, TIM-3, KIR, CD96, CTLA-4, BTLA, SRPc, or CD200, wherein a complementary binding target of the first protein binding moiety is disposed on a surface of a tumor cell.

10. The engineered extracellular vesicle of claim 7 wherein the type II membrane protein is 4-1BBL, CD70, GITRL, CD40L, CD30L, or TL1A, wherein a complementary binding target of the second protein binding moiety is disposed on a surface of an immune cell.

11. The engineered extracellular vesicle of claim 1 wherein the transmembrane domain of the first exosomal membrane protein is from Platelet Derived Growth Factor Receptor (PDGFR), and the second exosomal membrane protein is CD9.

12. The engineered extracellular vesicle of claim 1, wherein each of the first fusion protein and the second fusion protein further comprise one or more epitope tags and one or more linker moieties.

13. The engineered extracellular vesicle of claim 3 wherein each of the first antibody moiety and the second antibody moiety is a single chain variable fragment (scFv).

14. The engineered extracellular vesicle of claim 13 wherein the first antibody scFv binds to a immune cell surface-marker protein, wherein the immune cell surface-marker protein is CD3; and
the second antibody scFv binds to a cancer cell surface-marker protein, wherein the cancer cell surface-marker protein is epidermal growth factor receptor (EGFR); or
the first antibody scFv binds to a cancer cell surface-marker protein, wherein the cancer cell surface-marker protein is epidermal growth factor receptor (EGFR); and
the second antibody scFv binds to an immune cell surface-marker protein, wherein the immune cell surface-marker protein is CD3.

15. The engineered extracellular vesicle of claim 14 wherein the first protein binding moiety is PD-1, and the second protein binding moiety is OX40L; or
the first protein binding moiety is OX40L, and the second protein binding moiety is PD-1.

16. The engineered extracellular vesicle of claim 1 wherein the first fusion protein comprises a formula T₁-A-L₁-B-L₂-C-T₂, wherein T1 is a first epitope tag, A is the first antibody moiety, L₁ is a first linker moiety, B is the second antibody moiety, L₂ is a second linker moiety, C is the first exosomal protein transmembrane domain, and T₂ is a second epitope tag; and
the second fusion protein comprises a formula T₃-D-L3-E-L4-F, wherein T3 is a third epitope tag, D is the first protein binding moiety, L₃ is a third linker moiety, E is the second exosomal membrane protein transmembrane domain, L₄ is a fourth linker moiety, and F is the second protein binding moiety.

17. The engineered extracellular vesicle of claim 16 wherein the first fusion protein has an amino acid sequence according to SEQ ID NO: 1 and the second fusion protein has an amino acid sequence according to SEQ ID NO: 2.

18. The engineered extracellular vesicle of claim 1 wherein the engineered extracellular vesicle has a particle size of about 25 nm to about 150 nm.

19. A composition comprising the engineered extracellular vesicle of claim 1; and a pharmaceutically acceptable carrier.

20. A method of treating triple negative breast cancer (TNBC) comprising:
administering to a subject in need thereof an effective amount of the composition of claim 19, wherein the composition treats the TNBC.
